(19) United States
(12) Patent Application Publication
(10) Pub. No.: US 2014/0228303 A1
(43) Pub. Date: Aug. 14, 2014

(54) NOVEL SGLT INHIBITORS

(71) Applicants: Rajesh Jain, New Delhi (IN); Sanjay Trehan, New Delhi (IN); Jagattaran Das, New Delhi (IN); Gurmeet Kaur Nanda, New Delhi (IN); Sastry V. R. S Thungathurthi, New Delhi (IN); Nishan Singh, New Delhi (IN); Sudhir Kumar Sharma, New Delhi (IN)

(72) Inventors: Rajesh Jain, New Delhi (IN); Sanjay Trehan, New Delhi (IN); Jagattaran Das, New Delhi (IN); Gurmeet Kaur Nanda, New Delhi (IN); Sastry V. R. S Thungathurthi, New Delhi (IN); Nishan Singh, New Delhi (IN); Sudhir Kumar Sharma, New Delhi (IN)

(73) Assignee: Panacea Biotec Ltd, New Delhi (IN)

(21) Appl. No.: 14/126,268

(22) PCT Filed: Jun. 13, 2012
(86) PCT No.: PCT/IN12/00416

§ 371 Date: Mar. 14, 2014

(30) Foreign Application Priority Data
Jun. 13, 2011 (IN) 1676/DEL/2011

(51) Int. Cl. A61K 31/7048; A61K 31/70; C07H 19/01; C07H 7/04

(52) U.S. Cl. CPC A61K 31/7048; C07H 7/04; A61K 31/70; C07H 19/01
USPC 514/23; 536/1.11

(57) ABSTRACT

The present invention relates to novel compounds of Formula I, their pharmaceutically acceptable derivatives, tautomeric forms, isomers, polymorphs, prodrugs, metabolites, salts or solvates thereof. The invention also relates to the processes for the synthesis of novel compounds of Formula I, their pharmaceutically acceptable derivatives, tautomeric forms, isomers, polymorphs, prodrugs, metabolites, salts or solvates thereof. The present invention also provides pharmaceutical compositions comprising novel compounds of Formula I and methods of treating or preventing one or more conditions or diseases that may be regulated or normalized via inhibition of Sodium Glucose Cotransporter-2 (SGLT-2).

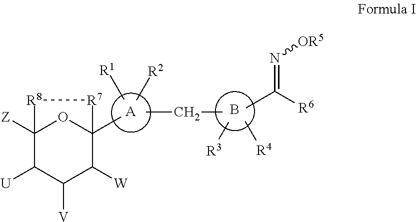

### RELATED APPLICATIONS

The present application claims priority from, Indian Application Number 1676/DEL/2011, filed Jun. 13, 2011.

### FIELD OF THE INVENTION

The present invention relates to novel compounds of Formula I, their pharmaceutically acceptable derivatives, tautomeric forms, isomers, polymorphs, prodrugs, metabolites, salts or solvates thereof. The invention also relates to the processes for the synthesis of novel compounds of Formula I, their pharmaceutically acceptable derivatives, tautomeric forms, isomers, polymorphs, prodrugs, metabolites, salts or solvates thereof. The present invention also provides pharmaceutical compositions comprising novel compounds of Formula I and methods of treating or preventing one or more conditions or diseases that may be regulated or normalized via inhibition of Sodium Glucose Cotransporter-2 (SGLT-2).

### BACKGROUND OF THE INVENTION

Diabetes is a metabolic disorder which is rapidly emerging as a global health care problem that threatens to reach pandemic levels. The number of people with diabetes worldwide is expected to rise from 285 million in 2010 to 438 million by 2030. Diabetes results from deficiency in insulin because of impaired pancreatic β-cell function or from resistance to insulin in body, thus leading to abnormally high levels of blood glucose.

Diabetes which results from complete deficiency in insulin secretion is Type 1 diabetes and the diabetes due to resistance to insulin activity together with an inadequate insulin secretion is Type 2 diabetes. Type 2 diabetes (Non insulin dependent diabetes) accounts for 90-95% of all diabetes. An early defect in Type 2 diabetes mellitus is insulin resistance which is a state of reduced responsiveness to circulating concentrations of insulin and is often present years before clinical diagnosis of diabetes. A key component of the pathophysiology of Type 2 diabetes mellitus involves an impaired pancreatic β-cell function which eventually contributes to decreased insulin secretion in response to elevated plasma glucose. The β-cell compensates for insulin resistance by increasing the insulin secretion, eventually resulting in reduced β-cell mass. Consequently, blood glucose levels stay at abnormally high levels (hyperglycemia).

Hyperglycemia is central to both the vascular consequences of diabetes and the progressive nature of the disease itself. Chronic hyperglycemia leads to decrease in insulin secretion and further to decrease in insulin sensitivity. As a result, the blood glucose concentration is increased, leading to diabetes, which is self-exacerbated. Chronic hyperglycemia has been shown to result in higher protein glycation, cell apoptosis and increased oxidative stress; leading to complications such as cardiovascular disease, stroke, nephropathy, retinopathy (leading to visual impairment or blindness), neuropathy, hypertension, dyslipidemia, premature atherosclerosis, diabetic foot ulcer and obesity. So, when a person suffers from diabetes, it becomes important to control the blood glucose level. Normalization of plasma glucose in Type 2 diabetes patients improves insulin action and may offset the development of beta cell failure and diabetic complications in the advanced stages of the disease.

Diabetes is basically treated by diet and exercise therapies. However, when sufficient relief is not obtained by these therapies, medicament is prescribed alongwith. Various anti-diabetic agents being currently used include biguanides (decrease glucose production in the liver and increase sensitivity to insulin), sulfonylureas and meglitinides (stimulate insulin production), α-glucosidase inhibitors (slow down starch absorption and glucose production) and thiazolidinediones (increase insulin sensitivity). These therapies have various side effects: biguanides cause lactic acidosis, sulfonylurea compounds cause significant hypoglycemia, α-glucosidase inhibitors cause abdominal bloating and diarrhea, and thiazolidinediones cause edema and weight gain. Recently introduced line of therapy includes inhibitors of dipeptidyl peptidase-IV (DPP-IV) enzyme, which may be useful in the treatment of diabetes, particularly in Type 2 diabetes. DPP-IV inhibitors lead to decrease in inactivation of incretins glucagon like peptide-1 (GLP-1) and gastric inhibitory peptide (GIP), thus leading to increased production of insulin by the pancreas in a glucose dependent manner. All of these therapies discussed, have an insulin dependent mechanism.

Another mechanism which offers insulin independent means of reducing glycemic levels, is the inhibition of sodium glucose co-transporters (SGLTs). In healthy individuals, almost 99% of the plasma glucose filtered in the kidneys is reabsorbed, thus leading to only less than 1% of the total filtered glucose being excreted in urine. Two types of SGLTs, SGLT-1 and SGLT-2, enable the kidneys to recover filtered glucose. SGLT-1 is a low capacity, high-affinity transporter expressed in the gut (small intestine epithelium), heart, and kidney (S3 segment of the renal proximal tubule), whereas SGLT-2 (a 672 amino acid protein containing 14 membrane-spanning segments), is a low affinity, high capacity glucose transporter, located mainly in the S1 segment of the proximal tubule of the kidney. SGLT-2 facilitates approximately 90% of glucose reabsorption and the rate of glucose filtration increases proportionally as the glycemic level increases. The inhibition of SGLT-2 should be highly selective, because non-selective inhibition leads to complications such as severe, sometimes fatal diarrhea, dehydration, peripheral insulin resistance, hypoglycemia in CNS and an impaired glucose uptake in the intestine.

Humans lacking a functional SGLT-2 gene appear to live normal lives, other than exhibiting copious glucose excretion with no adverse effects on carbohydrate metabolism. However, humans with SGLT-1 gene mutations are unable to transport glucose or galactose normally across the intestinal wall, resulting in condition known as glucose-galactose malabsorption syndrome.

Hence, competitive inhibition of SGLT-2, leading to renal excretion of glucose represents an attractive approach to normalize the high blood glucose associated with diabetes. Lower blood glucose levels would, in turn, lead to reduced rates of protein glycation, improved insulin sensitivity in liver and peripheral tissues, and improved cell function. As a consequence of progressive reduction in hepatic insulin resistance, the elevated hepatic glucose output which is characteristic of Type 2 diabetes would be expected to gradually diminish to normal values. In addition, excretion of glucose may reduce overall caloric load and lead to weight loss. Risk of hypoglycemia associated with SGLT-2 inhibition mechanism is low, because there is no interference with the normal counter regulatory mechanisms for glucose.

The first known non-selective SGLT-2 inhibitor was the natural product phlorizin (glucose, 1-[2-β-D-glucopyranosyloxy)-4,6-dihydroxyphenyl]-3-(4-hydroxyphenyl)-1-propanone). Subsequently, several other synthetic analogues were derived based on the structure of phlorizin. Optimisation of the scaffolds to achieve selective SGLT-2 inhibitors led to the discovery of several considerably different scaffolds.

C-glycoside derivatives have been disclosed, for example, in PCT publications WO2004013118, WO2005085265, WO2006008038, WO2006034489, WO2006037537, WO2006010557, WO2006089872, WO2006002912, WO2006054629, WO2006064033, WO2007136116, WO2007000445, WO2007093610, WO2008069327, WO2008020011, WO2008013321, WO2008013277, WO2008042688, WO2008122014, WO2008116195, WO2008042688, WO2009026537, WO2010147430, WO2010023594, WO2010022313, WO2011051864, WO2011048148 and WO2012019496 U.S. Pat. No. 6,515,117B2, U.S. Pat. No. 6,936,590B2 and U.S. Pat. No. 7,202,350B2 and Japanese patent application JP2004359630.

The compounds shown below are the SGLT-2 inhibitors which have reached advanced stages of human clinical trials: Bristol-Myers Squibb's “Dapagliflozin” with Formula A, Mitsubishi Tanabe and Johnson & Johnson's “Canagliflozin” with Formula B, Lexicon's “Lx-4211” with Formula C, Boehringer Ingelheim and Eli Lilly's “Empagliflozin” with Formula D, Roche and Chugai's “Tofogliflozin” with Formula E, Taisho's “Luseogliflozin” with Formula F, Pfizer's “Ertugliflozin” with Formula G and Astellas and Kotobuki's “Ipragliflozin” with Formula H.

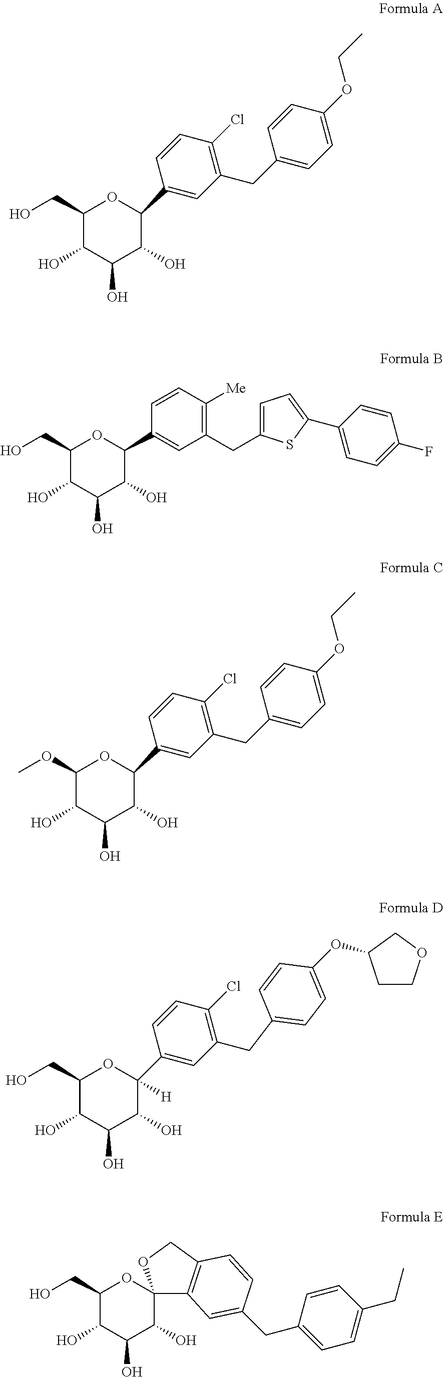

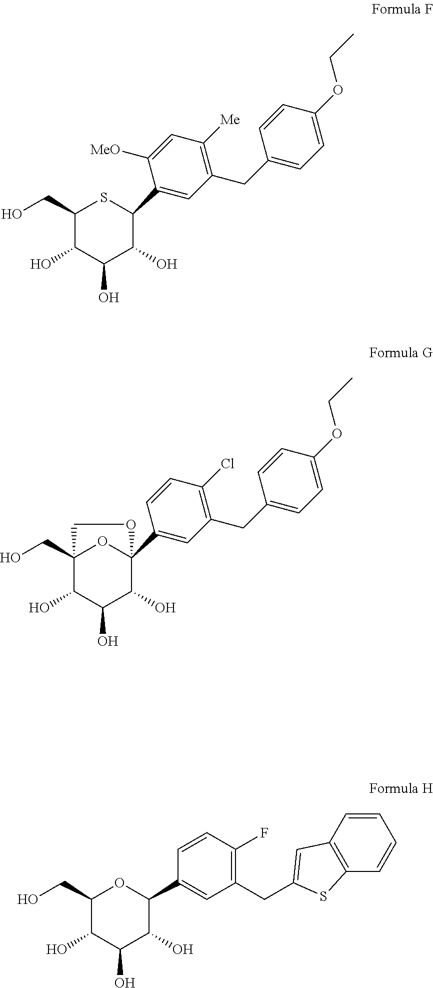 [SMILES: CCOc1ccc(Cc2cc([C@@H]3S[C@H](CO)[C@@H](O)[C@H](O)[C@H]3O)c(OC)cc2C)cc1 ; CCOc1ccc(Cc2cc([C@]34OC[C@](CO)(O3)[C@@H](O)[C@H](O)[C@H]4O)ccc2Cl)cc1 ; OC[C@H]1O[C@@H](c2ccc(F)c(Cc3cc4ccccc4s3)c2)[C@H](O)[C@@H](O)[C@@H]1O]

In spite of all these molecules in advanced stages of human clinical trials, there is still no drug available in the market as SGLT-2 inhibitor. Out of the potential candidates entering the clinical stages, many have been discontinued, emphasizing the unmet need. Thus there is an ongoing requirement to screen more scaffolds useful as SGLT-2 inhibitors that can have advantageous potency, stability, selectivity, better half-life, and/or better pharmacodynamic properties. In this regard, a novel class of SGLT-2 inhibitors is provided herein.

### SUMMARY OF THE INVENTION

The present invention is defined in the claims and relates to the novel compounds of Formula I,

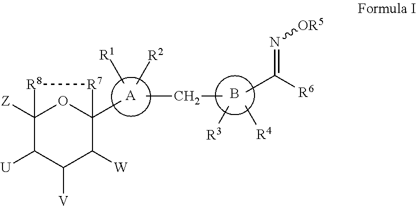

wherein:
‘---’ is either a single bond or absent;
ring A represents monocyclic or polycyclic C₃₋₂₀ cycloalkyl, C₆₋₁₀ aryl, 5-10 membered heteroaryl or 3-14 membered heterocyclyl ring;
ring B represents monocyclic or polycyclic C₃₋₂₀ cycloalkyl, C₆₋₁₀ aryl, 5-10 membered heteroaryl or 3-14 membered heterocyclyl ring;
U, V and W are independently selected from —H, C₁₋₁₂ alkyl, C₂₋₁₂ alkenyl, C₂₋₁₂ alkynyl, —OH, —CN, —N₃, —NO₂, —OCONH₂, —F, —Cl, —Br, —I, —COOH, —CONH₂, —CONHNH₂, —NH₂, —NHCONH₂, —NHCSNH₂, —NH(C═NH)NH₂, —NHNH₂, —NHCHO, —NHCOOH, —SH, —SO₃H, —CH(═NOH), —COR^{a}, —OR⁹, —COOR^{a}, —CONR^{a}R^{b}, —NR^{a}R^{b}, —NR^{a}SO₂R^{b}, —NR^{a}CONR^{b}R^{c}, —NR^{a}COR^{b}, —NR^{a}COOR^{b}, —OCOR^{a}, —OCOOR^{a}, —OCONR^{a}R^{b}, —SR^{a}, —S(O)R^{a}, —S(O)₂R^{a}, —SO₂NR^{a}R^{b}, —CR^{a} (═NOR^{b}), —NHP(O)R^{a}R^{b}; wherein the said C₁₋₁₂ alkyl, C₂₋₁₂ alkenyl and C₂₋₁₂ alkynyl, may optionally be substituted at any available position by one or more suitable substituents selected from R¹⁰;
   provided that at least two out of U, V and W represent —OR⁹;
Z represents —(CH₂)ₙOR^{a}, —OR^{a}, —OCOR^{a}, —OCOOR^{a}, —OCONR^{a}R^{b}, C₁₋₁₂ alkyl, C₂₋₁₂ alkenyl, C₂₋₁₂ alkynyl, —CN, —OCONH₂, —CHO, —COOH, —CONH₂, —CONHNH₂, —NH₂, —NHCOOH, —CH₂OH, —OH, —SH, —SO₃H, —CH(═NOH), —CH(═NCN), —COR^{a}, —COOR^{a}, —CONR^{a}R^{b}, —NR^{a}R^{b}, —NR^{a}SO₂R^{b}, —NR^{a}CONR^{b}R^{c}, —NR^{a}NR^{b}R^{c}, —NR^{a}COR^{b}, —NR^{a}COOR^{b}, —SR^{a}, —S(O)R^{a}, —S(O)₂R^{a}, —SO₂NR^{a}R^{b} or —CR^{a}(═NOR^{b}); wherein the said C₁₋₁₂ alkyl, C₂₋₁₂ alkenyl and C₂₋₁₂ alkynyl, may optionally be substituted at any available position by one or more suitable substituents selected from R¹⁰;
R¹, R², R³ and R⁴ are independently selected from —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCONH₂, —F, —Cl, —Br, —I, —CHO, —COOH, —CONH₂, —NH₂, —NHCONH₂, —NHCHO, —NHCOOH, —OH, —OR^{a}, —SH, —SO₃H; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁵ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, or 5-10 membered heteroaryl; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁶ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —NH₂, —NHCONH₂, —NHCHO, —OH, —SH, —NR^{a}R^{b}, —NR^{a}CONR^{b}R^{c}, —NR^{a}COR^{b}, —OR^{a} or —SR^{a}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁷ represents —H, —OH or —OR⁹;
R⁸ represents —H, —CHO, —COOH, —CONH₂, —OH, —CH(═NOH), —COR^{a}, —COOR^{a}, —CONR^{a}R^{b}, —CR^{a}(═NOR^{b}), —OR^{a}, or —(CH₂)ₙOR^{a};
   or
R⁷ and R⁸ can be joined together to form a saturated or unsaturated ring, in which one or more methylene groups or methyne groups can be replaced with O, S, NR^{a} or oxo; the ring thus formed may optionally be substituted at any available position by one or more suitable substituents selected from R¹¹;
   or
R⁸ and Z can be joined together to form a saturated or unsaturated ring, in which one or more methylene groups or methyne groups can be replaced with O, S, NR^{a} or oxo; the ring thus formed may optionally be substituted at any available position by one or more suitable substituents selected from R¹¹;
R⁹ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CHO, —CONH₂, —COR^{a}, —CONR^{a}R^{b}, —S(O)₂R^{a}, —SO₂NR^{a}R^{b}, —P(O)R^{a}R^{b}, —P(O)OR^{a}OR^{b}, —P(O)R^{a}OR^{b}, —P(O)NR^{a}OR^{b} or —P(O)NR^{a}R^{b}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CHO, —CONH₂, —COR^{a}, —CONR^{a}R^{b}, —S(O)₂R^{a}, —SO₂NR^{a}R^{b}, —P(O)R^{a}R^{b}, —P(O)OR^{a}OR^{b}, —P(O)R^{a}OR^{b}, —P(O)NR^{a}OR^{b}, —P(O)NR^{a}R^{b}; further wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹⁰ represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH), —CH(═NCN), —COR^{a}, —CSR^{a}, —COOR^{a}, —CSOR^{a}, —COSR^{a}, —CONR^{a}R^{b}, —CSNR^{a}R^{b}, —COCOR^{a}, —CONR^{a}NR^{b}R^{c}, —CSNR^{a}NR^{b}R^{c}, —CSNR^{a}R^{b}, —NR^{a}R^{b}, —NR^{a}SO₂R^{b}, —NR^{a}CONR^{b}R^{c}, —NR^{a}CSNR^{b}R^{c}, —NR^{a} (C═NR^{b})NR^{c}R^{d}, —NR^{a}NR^{b}R^{c}, —NR^{a}COR^{b}, —NR^{a}CSR^{b}, —NR^{a}COOR^{b}, —NR^{a}CSOR^{b}, —OR^{a}, —OCOR^{a}, —OCOOR^{a}, —OCONR^{a}R^{b}, —OCSR^{a}, —OCSOR^{a}, —ONO₂, —OCSNR^{a}R^{b}, —SR^{a}, —S(O)R^{a}, —S(O)₂R^{a}, —SO₂NR^{a}R^{b}, —CR^{a}(═NOR^{b}), —CR^{a}(═NCOOR^{b}), —CR^{a}(═NSOR^{b}), —CR^{a}(═NSO₂R^{b}), —C(═NR^{a})—NR^{b}R^{c}, —C(═NOR^{a})—NR^{b}R^{c}, —CR^{a}(═NCN), —NCR^{a}, —P(O)R^{a}R^{b}, —P(O)OR^{a}OR^{b}, —P(O)R^{a}OR^{b}, —P(O)NR^{a}OR^{b}, —P(O)NR^{a}R^{b}, —OP(O)R^{a}R^{b} or —NHP(O)R^{a}R^{b}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹¹ represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH) or —CH(═NCN); wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹² represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH) or —CH(═NCN);
R^{a}, R^{b}, R^{c} and R^{d} are independently selected from —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH), —CH(═NCN); each of which may optionally be substituted at any available position by one or more suitable substituents selected from R¹²;
   or
R^{a} and R^{b} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹;
   or
R^{b} and R^{c} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹;
   or
R^{c} and R^{d} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹;
   n represents 1, 2, 3, 4 or 5; their pharmaceutically acceptable derivatives, tautomeric forms, isomers, polymorphs, prodrugs, metabolites, salts or solvates thereof.

A further aspect of the present invention provides processes for the preparation of the novel compounds of Formula I, their pharmaceutically acceptable derivatives, tautomeric forms, isomers, polymorphs, prodrugs, metabolites, salts or solvates thereof.

Another aspect of the present invention provides pharmaceutical compositions, containing compounds of Formula I, their pharmaceutically acceptable derivatives, tautomeric forms, isomers, polymorphs, prodrugs, metabolites, salts or solvates thereof in combination with one or more pharmaceutically acceptable carrier(s), adjuvants and vehicles.

Another aspect of the present invention is the use of the compounds of Formula I, their pharmaceutically acceptable derivatives, tautomeric forms, isomers, polymorphs, prodrugs, metabolites, salts or solvates thereof, for the prophylaxis, amelioration and/or treatment of one or more condition(s)/disease(s)/disorder(s), in a subject in need thereof.

Still another aspect of the present invention is the use of the compounds of Formula I, their pharmaceutically acceptable derivatives, tautomeric forms, isomers, polymorphs, prodrugs, metabolites, salts or solvates thereof, for the prophylaxis, amelioration and/or treatment of one or more condition(s)/disease(s)/disorder(s) that may be regulated or normalized via inhibition of SGLT-2.

Yet another aspect of the invention is to provide methods of using the compounds of Formula I of the present invention or compositions comprising the compounds of Formula I for the prophylaxis, amelioration and/or treatment of disease(s)/disorder(s) involving SGLT-2 inhibition which comprises administering to a subject in need thereof the compounds of Formula I or compositions comprising a pharmaceutically effective amount of the compounds of Formula I.

A further aspect of the present invention is the use of a compound of Formula I for the manufacture of a medicament for the prophylaxis, amelioration and/or treatment of one or more condition(s)/disease(s)/disorder(s) involving SGLT-2 inhibition in a subject in need thereof.

The present invention also encompasses prodrugs and active metabolites of the compounds of the Formula I.

Other aspects of the invention will be set forth in the description which follows, and in part will be apparent from the description, or may be learnt by the practice of the invention.

### DETAILED DESCRIPTION OF THE INVENTION

The present invention is defined in the claims and relates to the novel compounds of Formula I,

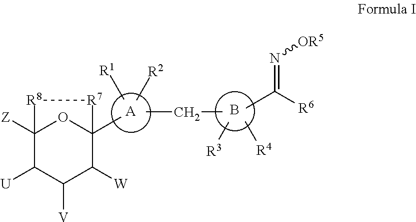

wherein:
‘---’ is either a single bond or absent;
ring A represents monocyclic or polycyclic C₃₋₂₀ cycloalkyl, C₆₋₁₀ aryl, 5-10 membered heteroaryl or 3-14 membered heterocyclyl ring;
ring B represents monocyclic or polycyclic C₃₋₂₀ cycloalkyl, C₆₋₁₀ aryl, 5-10 membered heteroaryl or 3-14 membered heterocyclyl ring;
U, V and W are independently selected from —H, C₁₋₁₂ alkyl, C₂₋₁₂ alkenyl, C₂₋₁₂ alkynyl, —OH, —CN, —N₃, —NO₂, —OCONH₂, —F, —Cl, —Br, —I, —COOH, —CONH₂, —CONHNH₂, —NH₂, —NHCONH₂, —NHCSNH₂, —NH(C═NH)NH₂, —NHNH₂, —NHCHO, —NHCOOH, —SH, —SO₃H, —CH(═NOH), —COR^{a}, —OR⁹, —COOR^{a}, —CONR^{a}R^{b}, —NR^{a}R^{b}, —NR^{a}SO₂R^{b}, —NR^{a}CONR^{b}R^{c}, —NR^{a}COR^{b}, —NR^{a}COOR^{b}, —OCOR^{a}, —OCOR^{a}, —OCONR^{a}R^{b}, —SR^{a}, —S(O)R^{a}, —S(O)₂R^{a}, —SO₂NR^{a}R^{b}, —CR^{a} (═NOR^{b}), —NHP(O)R^{a}R^{b}; wherein the said C₁₋₁₂ alkyl, C₂₋₁₂ alkenyl and C₂₋₁₂ alkynyl, may optionally be substituted at any available position by one or more suitable substituents selected from R¹⁰;
provided that at least two out of U, V and W represent —OR⁹;
Z represents —(CH₂)ₙOR^{a}, —OR^{a}, —OCOR^{a}, —OCOOR^{a}, —OCONR^{a}R^{b}, C₁₋₁₂ alkyl, C₂₋₁₂ alkenyl, C₂₋₁₂ alkynyl, —CN, —OCONH₂, —CHO, —COOH, —CONH₂, —CONHNH₂, —NH₂, —NHCOOH, —CH₂OH, —OH, —SH, —SO₃H, —CH(═NOH), —CH(═NCN), —COR^{a}, —COOR^{a}, —CONR^{a}R^{b}, —NR^{a}R^{b}, —NR^{a}SO₂R^{b}, —NR^{a}CONR^{b}R^{c}, —NR^{a}NR^{b}R^{c}, —NR^{a}COR^{b}, —NR^{a}COOR^{b}, —SR^{a}, —S(O)R^{a}, —S(O)₂R^{a}, —SO₂NR^{a}R^{b} or —CR^{a}(═NOR^{b}); wherein the said C₁₋₁₂ alkyl, C₂₋₁₂ alkenyl and C₂₋₁₂ alkynyl, may optionally be substituted at any available position by one or more suitable substituents selected from R¹⁰;
R¹, R², R³ and R⁴ are independently selected from —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCONH₂, —F, —Cl, —Br, —I, —CHO, —COOH, —CONH₂, —NH₂, —NHCONH₂, —NHCHO, —NHCOOH, —OH, —OR^{a}, —SH, —SO₃H; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁵ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, or 5-10 membered heteroaryl; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁶ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —NH₂, —NHCONH₂, —NHCHO, —OH, —SH, —NR^{a}R^{b}, —NR^{a}CONR^{b}R^{c}, —NR^{a}COR^{b}, —OR^{a} or —SR^{a}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁷ represents —H, —OH or —OR⁹;
R⁸ represents —H, —CHO, —COOH, —CONH₂, —OH, —CH(═NOH), —COR^{a}, —COOR^{a}, —CONR^{a}R^{b}, —CR^{a}(═NOR^{b}), —OR^{a}, or —(CH₂)ₙOR^{a};
   or
R⁷ and R⁸ can be joined together to form a saturated or unsaturated ring, in which one or more methylene groups or methyne groups can be replaced with O, S, NR^{a} or oxo; the ring thus formed may optionally be substituted at any available position by one or more suitable substituents selected from R¹¹;
   or
R⁸ and Z can be joined together to form a saturated or unsaturated ring, in which one or more methylene groups or methyne groups can be replaced with O, S, NR^{a} or oxo; the ring thus formed may optionally be substituted at any available position by one or more suitable substituents selected from R¹¹;
R⁹ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CHO, —CONH₂, —COR^{a}, —CONR^{a}R^{b}, —S(O)₂R^{a}, —SO₂NR^{a}R^{b}, —P(O)R^{a}R^{b}, —P(O)OR^{a}OR^{b}, —P(O)R^{a}OR^{b}, —P(O)NR^{a}OR^{b} or —P(O)NR^{a}R^{b}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CHO, —CONH₂, —COR^{a}, —CONR^{a}R^{b}, —S(O)₂R^{a}, —SO₂NR^{a}R^{b}, —P(O)R^{a}R^{b}, —P(O)OR^{a}OR^{b}, —P(O)R^{a}OR^{b}, —P(O)NR^{a}OR^{b}, —P(O)NR^{a}R^{b}; further wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹⁰ represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH), —CH(═NCN), —COR^{a}, —CSR^{a}, —COOR^{a}, —CSOR^{a}, —COSR^{a}, —CONR^{a}R^{b}, —CSNR^{a}R^{b}, —COCOR^{a}, —CONR^{a}NR^{b}R^{c}, —CSNR^{a}NR^{b}R^{c}, —CSNR^{a}R^{b}, —NR^{a}R^{b}, —NR^{a}SO₂R^{b}, —NR^{a}CONR^{b}R^{c}, —NR^{a}CSNR^{b}R^{c}, —NR^{a} (C═NR^{b})NR^{c}R^{d}, —NR^{a}NR^{b}R^{c}, —NR^{a}COR^{b}, —NR^{a}CSR^{b}, —NR^{a}COOR^{b}, —NR^{a}CSOR^{b}, —OR^{a}, —OCOR^{a}, —OCOOR^{a}, —OCONR^{a}R^{b}, —OCSR^{a}, —OCSOR^{a}, —ONO₂, —OCSNR^{a}R^{b}, —SR^{a}, —S(O)R^{a}, —S(O)₂R^{a}, —SO₂NR^{a}R^{b}, —CR^{a}(═NOR^{b}), —CR^{a}(═NCOOR^{b}), —CR^{a}(═NSOR^{b}), —CR^{a}(═NSO₂R^{b}), —C(═NR^{a})—NR^{b}R^{c}, —C(═NOR^{a})—NR^{b}R^{c}, —CR^{a}(═NCN), —NCR^{a}, —P(O)R^{a}R^{b}, —P(O)OR^{a}OR^{b}, —P(O)R^{a}OR^{b}, —P(O)NR^{a}OR^{b}, —P(O)NR^{a}R^{b}, —OP(O)R^{a}R^{b} or —NHP(O)R^{a}R^{b}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹¹ represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH) or —CH(═NCN); wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹² represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH) or —CH(═NCN);
R^{a}, R^{b}, R^{c} and R^{d} are independently selected from —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH), —CH(═NCN); each of which may optionally be substituted at any available position by one or more suitable substituents selected from R¹²;
   or
R^{a} and R^{b} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹;
   or
R^{b} and R^{c} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹;
   or
R^{c} and R^{d} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹;
   n represents 1, 2, 3, 4 or 5; their pharmaceutically acceptable derivatives, tautomeric forms, isomers, polymorphs, prodrugs, metabolites, salts or solvates thereof.

One embodiment of the present invention provides compounds of Formula Ia,

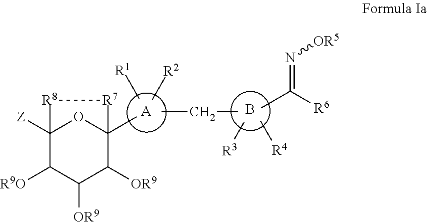

wherein:
Z, R¹, R², R³, R⁴, R⁵, R⁶, R⁷, R⁸, R⁹, ring A and ring B are as defined herein, R⁹ is preferably hydrogen; their pharmaceutically acceptable derivatives, tautomeric forms, isomers, polymorphs, prodrugs, metabolites, salts or solvates thereof.

Still another embodiment of the present invention provides compounds of Formula Ib,

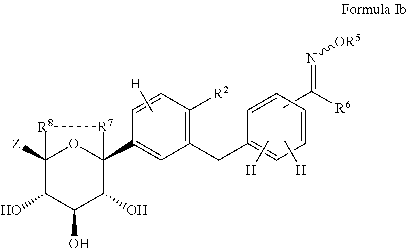

wherein:
Z, R¹, R², R³, R⁴, R⁵, R⁶, R⁷ and R⁸ are as defined herein; preferably R² is Cl, F, CH₃, H, CN, cyclopropyl or ethynyl; their pharmaceutically acceptable derivatives, tautomeric forms, isomers, polymorphs, prodrugs, metabolites, salts or solvates thereof.

A further embodiment of the present invention provides compounds of Formula Ic,

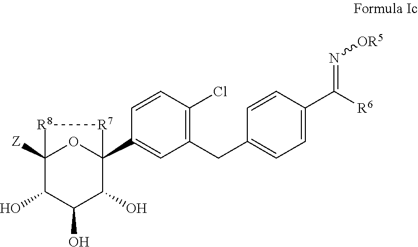

wherein:
Z, R⁵, R⁶, R⁷ and R⁸ are as defined herein; their pharmaceutically acceptable derivatives, tautomeric forms, isomers, polymorphs, prodrugs, metabolites, salts or solvates thereof.

In another embodiment of the compounds of the present invention, it is preferred that Z is selected from —(CH₂)ₙOR^{a} or —OR^{a}; preferably R⁵ represents —H, C₁₋₁₂ alkyl or C₆₋₁₀ aryl and R⁶ preferably represents —H, C₁₋₁₂ alkyl, —NR^{a}R^{b}, C₆₋₁₀ aryl or 5-10 membered heteroaryl.

In still another embodiment of the compounds of the present invention, it is preferred that
i. R⁷ and R⁸ are joined together to form a saturated ring, in which one or more methylene groups can be replaced with O; wherein the ring thus formed is unsubstituted or substituted at any available position by one or more suitable substituents selected from R¹¹; or
ii. R⁷ is H and R⁸ is H; or
iii. R⁷ is —OCH₃ and R⁸ is H.

Another embodiment of the present invention provides compounds of Formula Id,

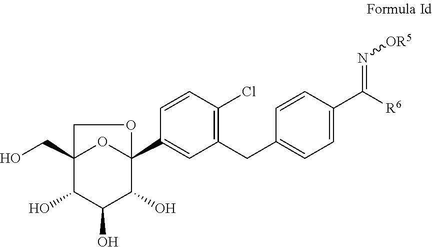

wherein:
R⁵ and R⁶ are as defined herein; their pharmaceutically acceptable derivatives, tautomeric forms, isomers, polymorphs, prodrugs, metabolites, salts or solvates thereof.

A still another embodiment of the present invention provides compounds of Formula Ie,

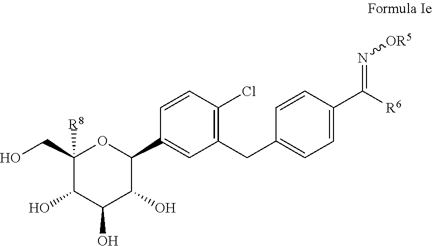

wherein:
R⁵, R⁶ and R⁸ are as defined herein; preferably R⁸ is H or —CH₂OH; their pharmaceutically acceptable derivatives, tautomeric forms, isomers, polymorphs, prodrugs, metabolites, salts or solvates thereof.

A further embodiment of the present invention provides compounds of Formula If,

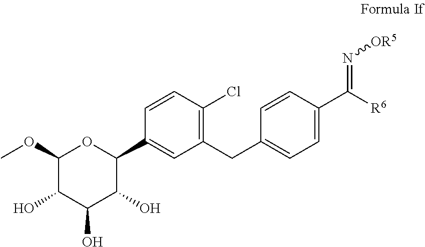

wherein:
R⁵ and R⁶ are as defined herein; their pharmaceutically acceptable derivatives, tautomeric forms, isomers, polymorphs, prodrugs, metabolites, salts or solvates thereof.

### DEFINITIONS

Relative to the above description of the compounds of the present invention, the following definitions apply.

The term “alkyl” as used herein alone or as part of another group refers to a straight or branched chain aliphatic hydrocarbon chain, having from 1 to 12 carbon atoms. Examples of alkyl include, but are not limited to methyl, ethyl, n-propyl, isopropyl, n-butyl, n-pentyl, t-butyl and the like. Alkyl groups may further be substituted with one or more suitable substituents.

The term “alkenyl” as used herein alone or as part of another group refers to a straight or branched chain aliphatic hydrocarbon group containing at least one carbon-carbon double bond, having from 2 to 12 carbon atoms. Examples of alkenyl include, but are not limited to ethenyl, 1-propenyl, 2-propenyl, iso-propenyl, 1-butenyl, 2-butenyl, and the like. Alkenyl groups may further be substituted with one or more suitable substituents.

The term “alkynyl” as used herein alone or as part of another group refers to a straight or branched chain aliphatic hydrocarbon group containing at least one carbon-carbon triple bond, having from 2 to 12 carbon atoms. Examples of alkynyl include, but are not limited to ethynyl, propynyl, and butynyl. Alkynyl groups may further be substituted with one or more suitable substituents.

The term “cycloalkyl” refers to cyclic alkyl groups constituting of 3 to 20 carbon atoms having a single cyclic ring or multiple condensed rings, for example, fused or spiro systems, unless otherwise constrained by the definition. Such cycloalkyl groups include, by way of example, single ring structures, for example, cyclopropyl, cyclobutyl, cyclopentyl, cyclohexyl, cyclooctyl, and the like, or multiple ring structures, for example, adamantyl, and bicyclo[2.2.1]heptane, or cyclic alkyl groups to which is fused an aryl group or another cycloalkyl group, for example, indane and the like. Cycloalkyl groups may further be substituted with one or more suitable substituents. The term “cycloalkyl” may optionally contain one or more unsaturated bonds.

The term “aryl” herein refers to six to ten membered monocyclic aromatic group, for example phenyl or naphthyl ring and the like optionally substituted with one or more suitable substituents. The aryl group may optionally be fused with one or two cycloalkyl group(s) or other aryl group(s) resulting in polycyclic ring system. The fused group may be further substituted with one or more suitable substituents.

The term “heteroaryl” unless and otherwise specified refers to a five to ten membered aromatic monocyclic ring structure, containing one to five heteroatoms independently selected from N, O, S or P. “Heteroaryl” also includes, but is not limited to, bicyclic or tricyclic rings, wherein the above defined heteroaryl ring is fused to one or two rings independently selected from the group consisting of an aryl ring, a cycloalkyl ring, a heterocyclyl ring and another monocyclic heteroaryl ring. Examples of heteroaryl groups include, but are not limited to, oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, thienyl, isoxazolyl, triazinyl, furanyl, benzofuranyl, indolyl, benzothiazolyl, benzoxazolyl, imidazo[1,2-α]pyrimidine, imidazo[1,2-a]pyrazine, tetrahydroquinoline and the like. The heteroaryl group may be further substituted at any available position with one or more suitable substituents. Point of attachment of heteroaryl group to another group may be through carbon or heteroatom.

The term “heterocyclyl” unless otherwise specified refers to a non-aromatic 3 to 14 membered monocyclic cycloalkyl group, fully or partially unsaturated, with one to five heteroatoms independently selected from N, O, S or P. “Heterocyclyl” also includes, but is not limited to, bicyclic or tricyclic rings, wherein the heterocyclyl ring is fused to one or two rings independently selected from the group consisting of an aryl ring, a cycloalkyl ring, a heteroaryl ring or heterocyclyl ring. The heterocyclyl group may be further substituted at any available position with one or more suitable substituents. Examples of heterocyclyl groups include but are not limited to, morpholinyl, oxazolidinyl, tetrahydrofuranyl, dihydrofuranyl, dihydropyridinyl, dihydroisooxazolyl, dihydrobenzofuryl, azabicyclohexyl, dihydroindonyl, piperidinyl or piperazinyl. Point of attachment of heterocyclyl group to another group may be through carbon or heteroatom.

“Halogen” refers to F, Cl, Br or I.
“Hydroxy” or “hydroxyl” refers to the group —OH.
The term “oxo” refers to carbonyl group represented as >C═O.

In all the above definitions, nitrogen, sulphur and phosphorus heteroatom can optionally be quaternerized or oxidized wherever permissible.

Examples of suitable substituents groups include, but are not limited to, alkyl, alkenyl, alkynyl, aryl, cycloalkyl, heterocyclyl, heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —N(C═NH)NH₂, —NHNH₂, —NHCHO, —NHCHS, —NHCOOH, —NHCSOH, —OH, —SH, —SO₃H, —CH(═NOH), —CH(═NCN) and the like.

The term “Protecting Group” or “PG” refers to a group which is in a modified form to preclude undesired side reactions at the protected site. The term protecting group, unless otherwise specified, may be used with groups, for example, hydroxy, amino, carboxy and examples of such groups are found in T. W. Greene. et al. “Protecting Groups in Organic Synthesis,” 3^{rd} Ed, Wiley, New York, which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule. Examples of suitable hydroxy and amino protecting groups include but are not limited to trimethylsilyl, triethylsilyl, o-nitrobenzyloxycarbonyl, p-nitrobenzyloxycarbonyl, t-butyldiphenylsilyl, t-butyldimethylsilyl, acetyl, trifluoroacetyl, benzyloxycarbonyl (CBz), t-butoxycarbonyl (Boc), 9-fluorenylmethylenoxycarbonyl (Fmoc), 2,2,2-trichloroethyloxycarbonyl, allyloxycarbonyl and the like. Examples of suitable carboxy protecting groups are benzhydryl, o-nitrobenzyl, p-nitrobenzyl, 2-naphthylmethyl, allyl, 2-chloroallyl, benzyl, 2,2,2-trichloroethyl, trimethylsilyl, t-butyldimethylsilyl, t-butyldiphenylsilyl, 2-(trimethylsilyl)ethyl, phenacyl, p-methoxybenzyl, acetonyl, p-methoxyphenyl, 4-pyridylmethyl, t-butyl and the like.

“Subject” includes humans, non-human mammals (e.g., dogs, cats, rabbits, cattle, horses, sheep and the like) or non-mammals (e.g., birds and the like).

The term “therapeutically effective amount” means the amount of a compound that, when administered to a subject for treating a disease, is sufficient to effect such treatment for the disease. The “therapeutically effective amount” will vary depending on the compound, the disease and its severity, weight, physical condition and responsiveness of the subject to be treated, among other factors.

A “pharmaceutically acceptable salt” refers to salts prepared from pharmaceutically acceptable non-toxic bases or acids including inorganic or organic bases and inorganic or organic acids. A “pharmaceutically acceptable salt” also encompasses any compound according to the present invention that is utilized in the form of a salt thereof, especially where the salt confers on a compound improved pharmacokinetic properties as compared to the free form of compound or a different salt form of the compound.

Term comprises/comprising and grammatical variations thereof when used in this specification are to be taken to specify the presence of stated features, integers, steps or components or groups thereof, but do not preclude the presence or addition of one or more other features, integers, steps, components or groups thereof.

Asymmetric centres may exist in the compounds of the present invention. The compounds of Formula I may have one or more stereogenic centres and so can exhibit optical isomerism. All such isomers including enantiomers, diastereomers, and epimers are included within the scope of this invention. Furthermore, the invention includes such compounds as single isomers (R and/or S) and as mixtures, including racemates. If desired, racemic mixtures of the compounds may be separated so that the individual enantiomers are isolated. The separation may be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. Starting materials of particular stereochemistry may either be commercially available or may be made by the methods described herein and resolved by techniques well known in the art. The independent syntheses of these diastereomers or their chromatographic separations may be achieved as known in the art by appropriate modifications.

Certain compounds according to Formula I, can also exist as tautomers, which have different points of attachment of hydrogen accompanied by one or more double bond shifts. These tautomers, either separately or as mixtures, are also considered to be within the scope of the invention.

The present invention also encompasses geometrical isomers of compounds of Formula I and the mixtures thereof. The geometrical isomers may exist in E or Z; Syn or anti configurations. These geometrical isomers, either separately or as mixtures, are also considered to be within the scope of the invention.

Certain compounds according to Formula I, may also exist as polymorphs. Various “polymorphs” of a compound of general Formula I forming part of this invention may be prepared by crystallization of a compound of Formula I under different conditions. For example, by using different solvents commonly used or their mixtures for recrystallization; crystallizations at different temperatures; various modes of cooling, ranging from very fast to very slow cooling during crystallizations, heating or melting the compound followed by gradual or fast cooling may also obtain polymorphs. The presence of polymorphs may be determined by solid probe NMR spectroscopy, IR spectroscopy, differential scanning calorimetry, powder X-ray diffraction or such other techniques.

Particularly useful examples of the present invention include but are not limited to the compounds selected from Table 1, including their pharmaceutically acceptable derivatives, tautomeric forms, isomers, polymorphs, prodrugs, metabolites, salts or solvates thereof:

TABLE 1

| Compound | |
|---|---|
| No | Structure |
| 1 | 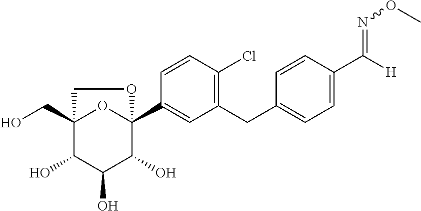 [SMILES: CON=Cc1ccc(Cc2cc([C@]34OC[C@](CO)(O3)[C@@H](O)[C@H](O)[C@H]4O)ccc2Cl)cc1] |
| 2 | 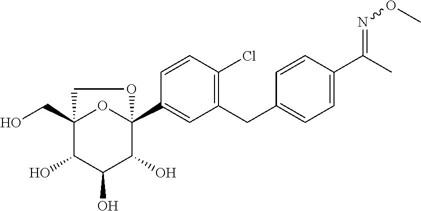 [SMILES: CON=C(C)c1ccc(Cc2cc([C@]34OC[C@](CO)(O3)[C@@H](O)[C@H](O)[C@H]4O)ccc2Cl)cc1] |
| 3 | 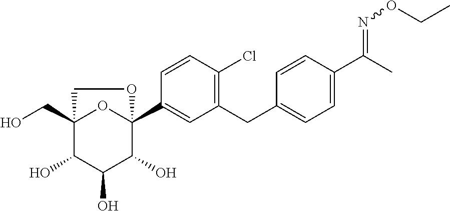 [SMILES: CCON=C(C)c1ccc(Cc2cc([C@]34OC[C@](CO)(O3)[C@@H](O)[C@H](O)[C@H]4O)ccc2Cl)cc1] |
| 4 | 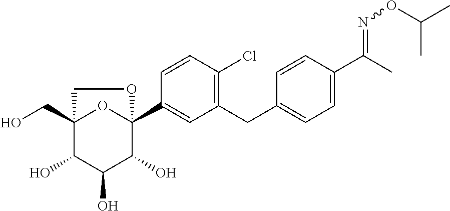 [SMILES: CC(=NOC(C)C)c1ccc(Cc2cc([C@]34OC[C@](CO)(O3)[C@@H](O)[C@H](O)[C@H]4O)ccc2Cl)cc1] |
| 5 | 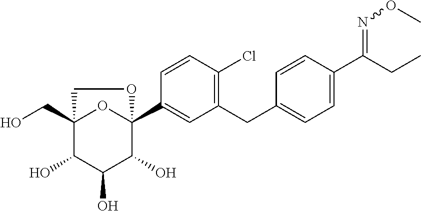 [SMILES: CCC(=NOC)c1ccc(Cc2cc([C@]34OC[C@](CO)(O3)[C@@H](O)[C@H](O)[C@H]4O)ccc2Cl)cc1] |
| 6 | 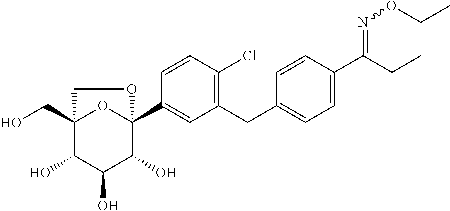 [SMILES: CCON=C(CC)c1ccc(Cc2cc([C@]34OC[C@](CO)(O3)[C@@H](O)[C@H](O)[C@H]4O)ccc2Cl)cc1] |
| 7 | 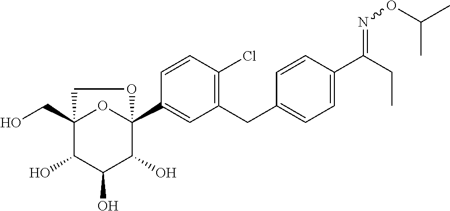 [SMILES: CCC(=NOC(C)C)c1ccc(Cc2cc([C@]34OC[C@](CO)(O3)[C@@H](O)[C@H](O)[C@H]4O)ccc2Cl)cc1] |
| 8 | 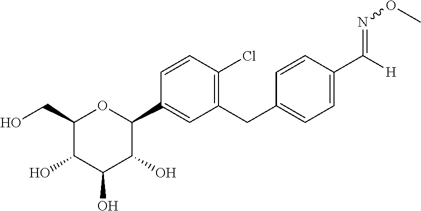 [SMILES: CON=Cc1ccc(Cc2cc([C@@H]3O[C@H](CO)[C@@H](O)[C@H](O)[C@H]3O)ccc2Cl)cc1] |
| 9 | 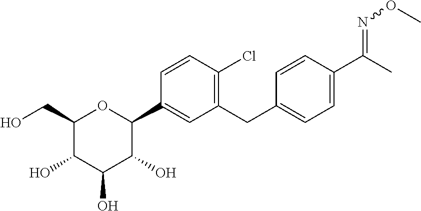 [SMILES: CON=C(C)c1ccc(Cc2cc([C@@H]3O[C@H](CO)[C@@H](O)[C@H](O)[C@H]3O)ccc2Cl)cc1] |
| 10 | 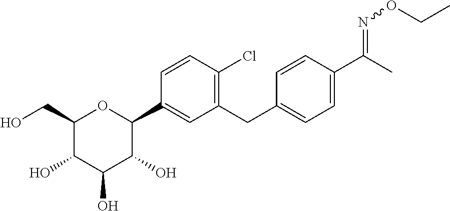 [SMILES: CCON=C(C)c1ccc(Cc2cc([C@@H]3O[C@H](CO)[C@@H](O)[C@H](O)[C@H]3O)ccc2Cl)cc1] |
| 11 | 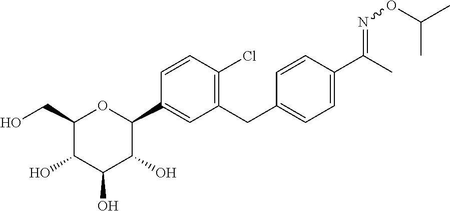 [SMILES: CC(=NOC(C)C)c1ccc(Cc2cc([C@@H]3O[C@H](CO)[C@@H](O)[C@H](O)[C@H]3O)ccc2Cl)cc1] |
| 12 | 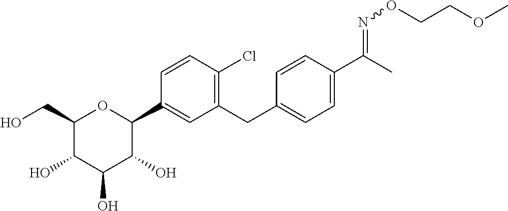 [SMILES: COCCON=C(C)c1ccc(Cc2cc([C@@H]3O[C@H](CO)[C@@H](O)[C@H](O)[C@H]3O)ccc2Cl)cc1] |
| 13 | 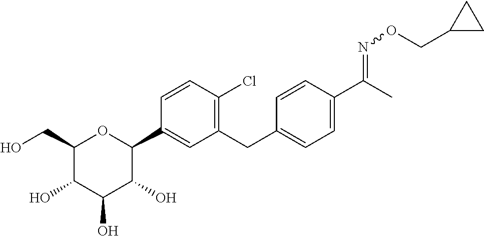 [SMILES: CC(=NOCC1CC1)c1ccc(Cc2cc([C@@H]3O[C@H](CO)[C@@H](O)[C@H](O)[C@H]3O)ccc2Cl)cc1] |
| 14 | 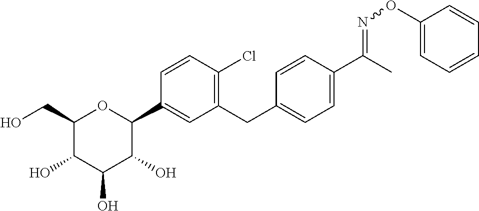 [SMILES: CC(=NOc1ccccc1)c1ccc(Cc2cc([C@@H]3O[C@H](CO)[C@@H](O)[C@H](O)[C@H]3O)ccc2Cl)cc1] |
| 15 | 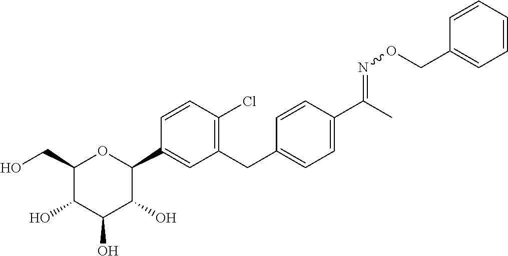 [SMILES: CC(=NOCc1ccccc1)c1ccc(Cc2cc([C@@H]3O[C@H](CO)[C@@H](O)[C@H](O)[C@H]3O)ccc2Cl)cc1] |
| 16 | 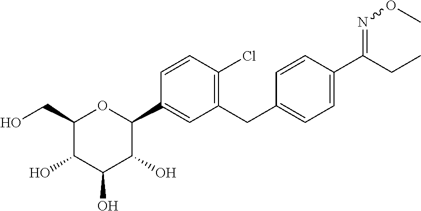 [SMILES: CCC(=NOC)c1ccc(Cc2cc([C@@H]3O[C@H](CO)[C@@H](O)[C@H](O)[C@H]3O)ccc2Cl)cc1] |
| 17 | 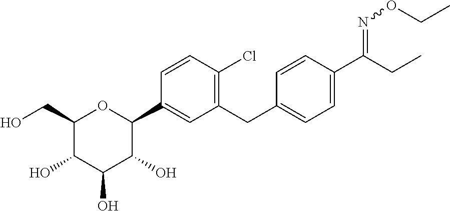 [SMILES: CCON=C(CC)c1ccc(Cc2cc([C@@H]3O[C@H](CO)[C@@H](O)[C@H](O)[C@H]3O)ccc2Cl)cc1] |
| 18 | 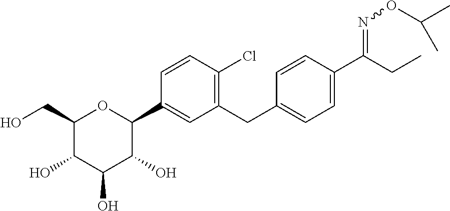 [SMILES: CCC(=NOC(C)C)c1ccc(Cc2cc([C@@H]3O[C@H](CO)[C@@H](O)[C@H](O)[C@H]3O)ccc2Cl)cc1] |
| 19 | 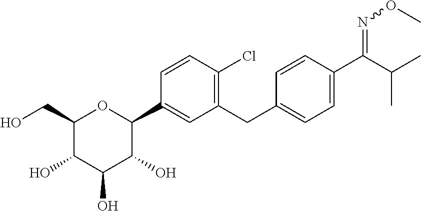 [SMILES: CON=C(c1ccc(Cc2cc([C@@H]3O[C@H](CO)[C@@H](O)[C@H](O)[C@H]3O)ccc2Cl)cc1)C(C)C] |
| 20 | 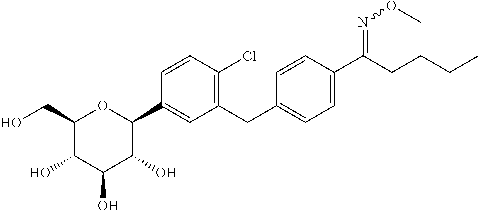 [SMILES: CCCCC(=NOC)c1ccc(Cc2cc([C@@H]3O[C@H](CO)[C@@H](O)[C@H](O)[C@H]3O)ccc2Cl)cc1] |
| 21 | 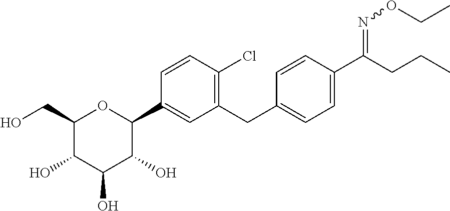 [SMILES: CCCC(=NOCC)c1ccc(Cc2cc([C@@H]3O[C@H](CO)[C@@H](O)[C@H](O)[C@H]3O)ccc2Cl)cc1] |
| 22 | 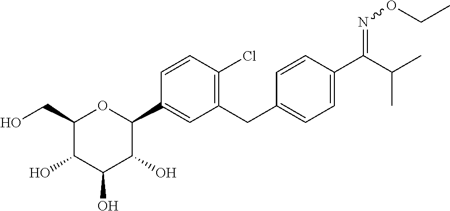 [SMILES: CCON=C(c1ccc(Cc2cc([C@@H]3O[C@H](CO)[C@@H](O)[C@H](O)[C@H]3O)ccc2Cl)cc1)C(C)C] |
| 23 | 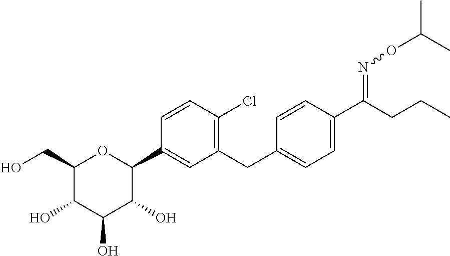 [SMILES: CCCC(=NOC(C)C)c1ccc(Cc2cc([C@@H]3O[C@H](CO)[C@@H](O)[C@H](O)[C@H]3O)ccc2Cl)cc1] |
| 24 | 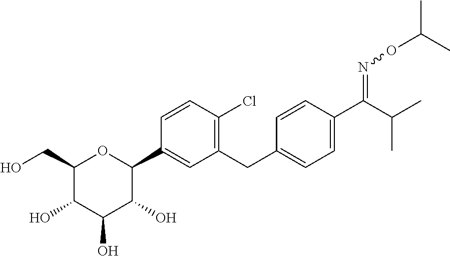 [SMILES: CC(C)ON=C(c1ccc(Cc2cc([C@@H]3O[C@H](CO)[C@@H](O)[C@H](O)[C@H]3O)ccc2Cl)cc1)C(C)C] |
| 25 | 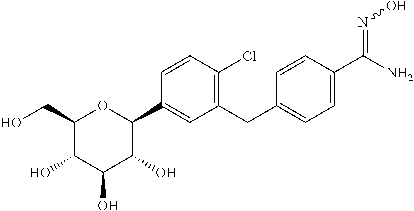 [SMILES: NC(=NO)c1ccc(Cc2cc([C@@H]3O[C@H](CO)[C@@H](O)[C@H](O)[C@H]3O)ccc2Cl)cc1] |
| 26 | 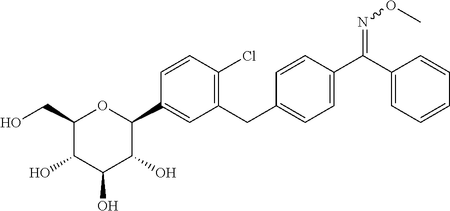 [SMILES: CON=C(c1ccccc1)c1ccc(Cc2cc([C@@H]3O[C@H](CO)[C@@H](O)[C@H](O)[C@H]3O)ccc2Cl)cc1] |
| 27 | 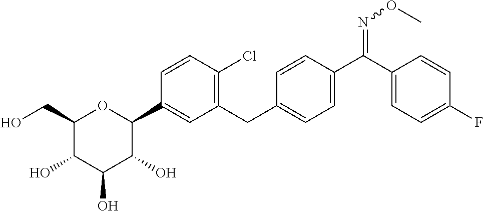 [SMILES: CON=C(c1ccc(F)cc1)c1ccc(Cc2cc([C@@H]3O[C@H](CO)[C@@H](O)[C@H](O)[C@H]3O)ccc2Cl)cc1] |
| 28 | 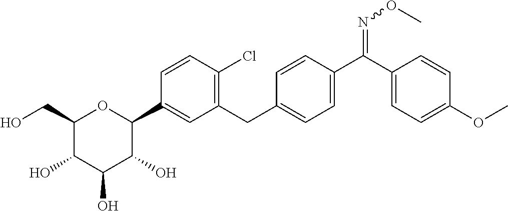 [SMILES: CON=C(c1ccc(Cc2cc([C@@H]3O[C@H](CO)[C@@H](O)[C@H](O)[C@H]3O)ccc2Cl)cc1)c1ccc(OC)cc1] |
| 29 | 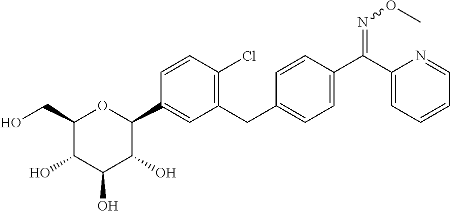 [SMILES: CON=C(c1ccc(Cc2cc([C@@H]3O[C@H](CO)[C@@H](O)[C@H](O)[C@H]3O)ccc2Cl)cc1)c1ccccn1] |
| 30 | 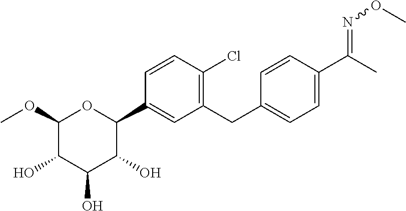 |
| 31 | 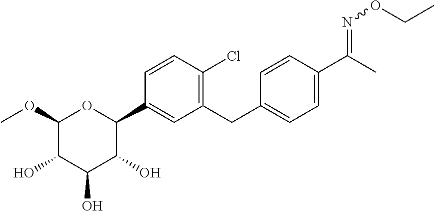 [SMILES: CCON=C(C)c1ccc(Cc2cc([C@@H]3O[C@H](OC)[C@@H](O)[C@H](O)[C@H]3O)ccc2Cl)cc1] |
| 32 | 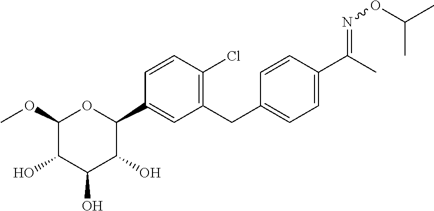 [SMILES: CO[C@H]1O[C@@H](c2ccc(Cl)c(Cc3ccc(C(C)=NOC(C)C)cc3)c2)[C@H](O)[C@@H](O)[C@@H]1O] |
| 33 | 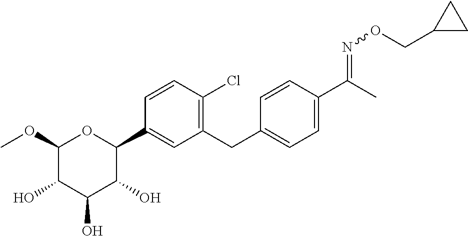 [SMILES: CO[C@H]1O[C@@H](c2ccc(Cl)c(Cc3ccc(C(C)=NOCC4CC4)cc3)c2)[C@H](O)[C@@H](O)[C@@H]1O] |
| 34 | 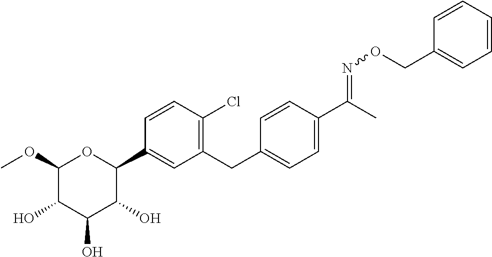 [SMILES: CO[C@H]1O[C@@H](c2ccc(Cl)c(Cc3ccc(C(C)=NOCc4ccccc4)cc3)c2)[C@H](O)[C@@H](O)[C@@H]1O] |
| 35 | 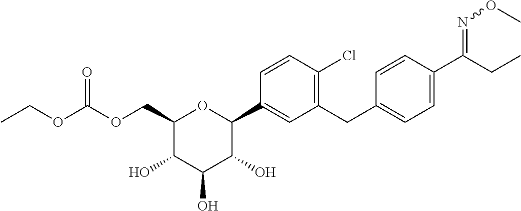 [SMILES: CCOC(=O)OC[C@H]1O[C@@H](c2ccc(Cl)c(Cc3ccc(C(CC)=NOC)cc3)c2)[C@H](O)[C@@H](O)[C@@H]1O] |
| 36 | 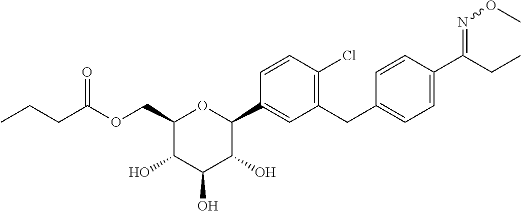 [SMILES: CCCC(=O)OC[C@H]1O[C@@H](c2ccc(Cl)c(Cc3ccc(C(CC)=NOC)cc3)c2)[C@H](O)[C@@H](O)[C@@H]1O] |
| 37 | 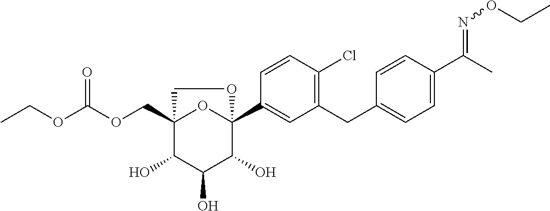 [SMILES: CCON=C(C)c1ccc(Cc2cc([C@]34OC[C@](COC(=O)OCC)(O3)[C@@H](O)[C@H](O)[C@H]4O)ccc2Cl)cc1] |
| 38 | 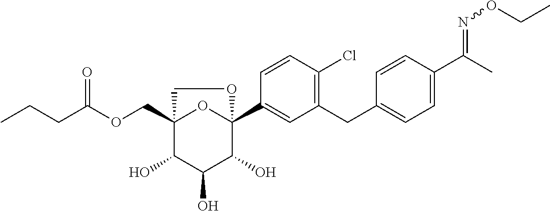 [SMILES: CCCC(=O)OC[C@@]12CO[C@@](c3ccc(Cl)c(Cc4ccc(C(C)=NOCC)cc4)c3)(O1)[C@H](O)[C@@H](O)[C@@H]2O] |
| 39 | 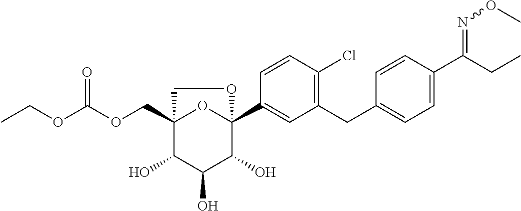 [SMILES: CCOC(=O)OC[C@@]12CO[C@@](c3ccc(Cl)c(Cc4ccc(C(CC)=NOC)cc4)c3)(O1)[C@H](O)[C@@H](O)[C@@H]2O] |
| 40 | 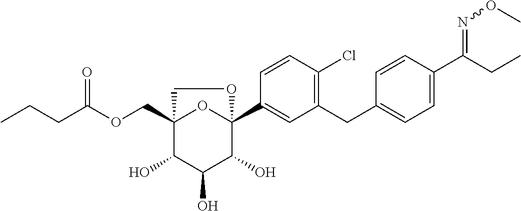 [SMILES: CCCC(=O)OC[C@@]12CO[C@@](c3ccc(Cl)c(Cc4ccc(C(CC)=NOC)cc4)c3)(O1)[C@H](O)[C@@H](O)[C@@H]2O] |
| 41 | 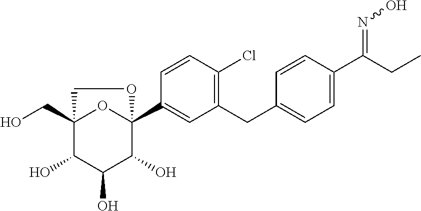 [SMILES: CCC(=NO)c1ccc(Cc2cc([C@]34OC[C@](CO)(O3)[C@@H](O)[C@H](O)[C@H]4O)ccc2Cl)cc1] |
| 42 | 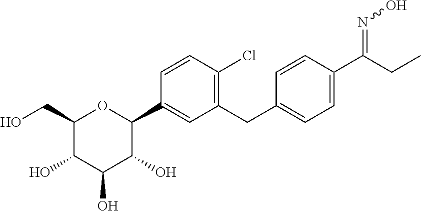 [SMILES: CCC(=NO)c1ccc(Cc2cc([C@@H]3O[C@H](CO)[C@@H](O)[C@H](O)[C@H]3O)ccc2Cl)cc1] |
| 43 | 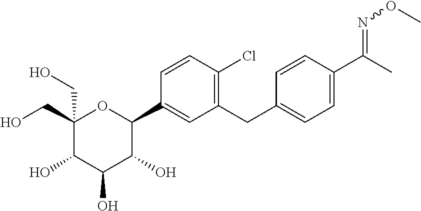 |
| 44 | 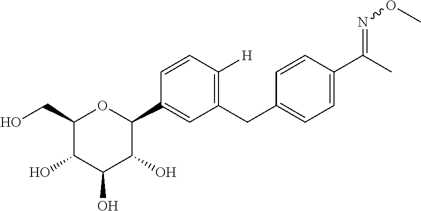 [SMILES: CON=C(C)c1ccc(Cc2cccc([C@@H]3O[C@H](CO)[C@@H](O)[C@H](O)[C@H]3O)c2)cc1] |
| 45 | 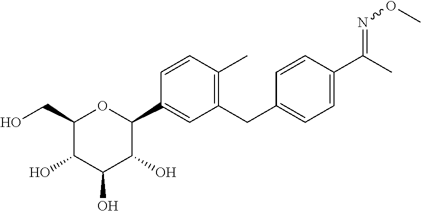 [SMILES: CON=C(C)c1ccc(Cc2cc([C@@H]3O[C@H](CO)[C@@H](O)[C@H](O)[C@H]3O)ccc2C)cc1] |
| 46 | 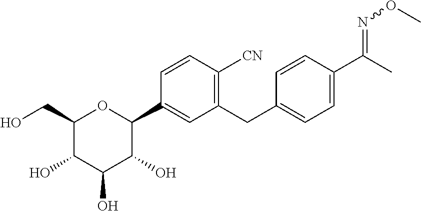 [SMILES: CON=C(C)c1ccc(Cc2cc([C@@H]3O[C@H](CO)[C@@H](O)[C@H](O)[C@H]3O)ccc2C#N)cc1] |
| 47 | 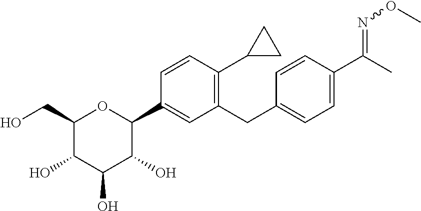 [SMILES: CON=C(C)c1ccc(Cc2cc([C@@H]3O[C@H](CO)[C@@H](O)[C@H](O)[C@H]3O)ccc2C2CC2)cc1] |
| 48 | 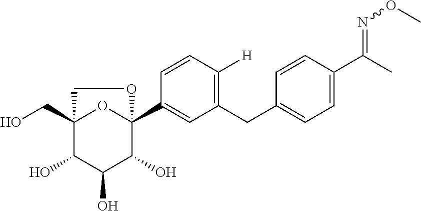 [SMILES: CON=C(C)c1ccc(Cc2cccc([C@]34OC[C@](CO)(O3)[C@@H](O)[C@H](O)[C@H]4O)c2)cc1] |
| 49 | 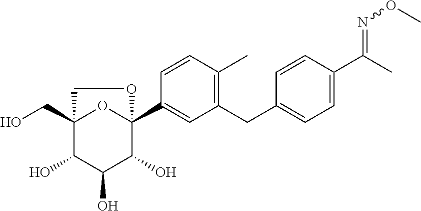 [SMILES: CON=C(C)c1ccc(Cc2cc([C@]34OC[C@](CO)(O3)[C@@H](O)[C@H](O)[C@H]4O)ccc2C)cc1] |
| 50 | 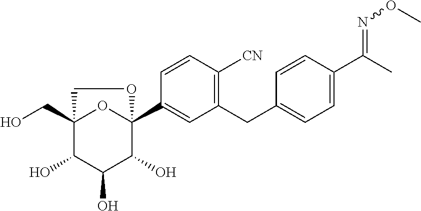 [SMILES: CON=C(C)c1ccc(Cc2cc([C@]34OC[C@](CO)(O3)[C@@H](O)[C@H](O)[C@H]4O)ccc2C#N)cc1] |

The compounds of the present invention may be prepared by the following reaction sequences as depicted in for example Scheme No 1 to 9. The compounds disclosed may also be prepared by techniques known in the art and familiar to the skilled organic chemist. All of the starting materials are either commercially available or can be prepared by procedures that would be well known to one of ordinary skill in organic chemistry.

“L” is used to denote an appropriate leaving group and as such may vary in nature depending on the exact reaction conditions employed. Some typical leaving groups may be fluoro, chloro, bromo, iodo, tosylate, mesylate, triflate and the like, but these should not be construed as limiting as many other leaving groups are also well known to those skilled in the art.

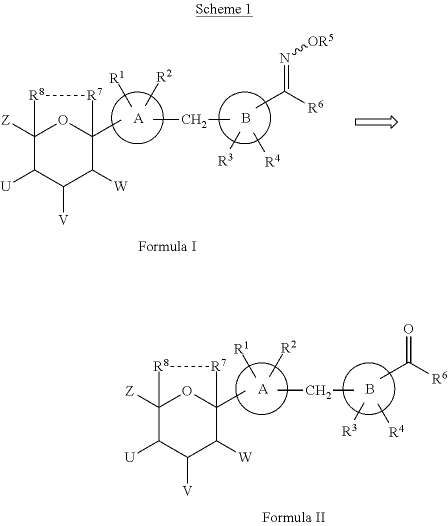

The compounds of the Formula I can be prepared from the compounds of Formula II as shown in the Scheme 1 by treating with compounds of Formula III (R⁵ONH₂) or their salts in the presence of a suitable base such as K₂CO₃, Na₂CO₃, NaHCO₃, pyridine, DIPEA, NEt₃ and the like in a solvent selected from but not limited to methanol, ethanol, pyridine, toluene, benzene or a combination thereof; optionally in the presence of NaOAc or ZnO.

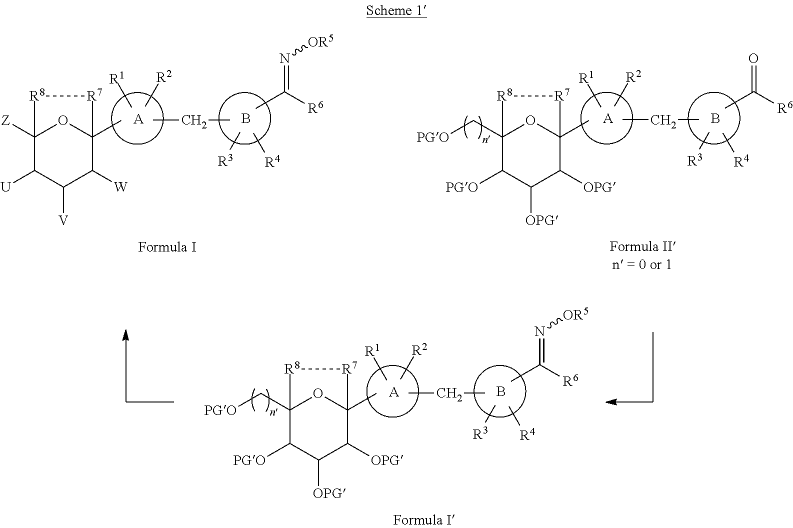

Alternately, compounds of Formula I can be prepared from compounds of Formula II′ in a two step procedure as described in Scheme 1′. The compounds of Formula II′ wherein U, V and/or W represent suitably protected hydroxyl groups, for example but not limited to O-acetyl, O-benzyl, O-allyl, O-p-methoxybenzyl and O-silyl, the oxime formation step described above can be followed by a suitable deprotection step to obtain the compounds of Formula I. Depending upon the nature of the protecting group of the compounds of Formula I′ (Novel Intermediates), deprotection method can be chosen by person skilled in the art. Such deprotection methods include but not limited to, treatment with alkali hydroxide, H₂/Pd—C, TMS1, BCl₃, pTSA, TFA, HCl, H₂SO₄, HBr, HI, TBAF, HF, DDQ, CAN, OsO₄, NaIO₄ and Pd(PPh₃)₄.

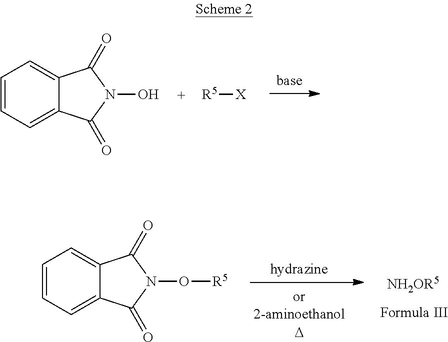

The commercially non available hydroxylamine derivatives of Formula III (NH₂OR⁵) can be prepared by following Scheme 2. Alkylation of N-hydroxyphthalimide can be done using R⁵—X, wherein X can be halogen or a leaving group for example but not limited to mesylate or tosylate in the presence of a base such as sodium hydride, potassium carbonate, LDA, potassium tert-butoxide, sodium tert-butoxide, LiHMDS and the like in a suitable solvent such as but not limited to THF, DMF, DMSO, acetone or a combination thereof. These types of transformations can be performed by methods as described in U.S. Pat. No. 5,120,849 and Chem. Pharm. Bull, 2003, 51, 138-151. The alkylated hydroxy phthalimide intermediate can be converted to compounds of Formula III by treating with reagents such as hydrazine hydrate, 2-aminoethanol and the like in the presence of a suitable solvent for example but not limited to methanol, ethanol or isopropanol.

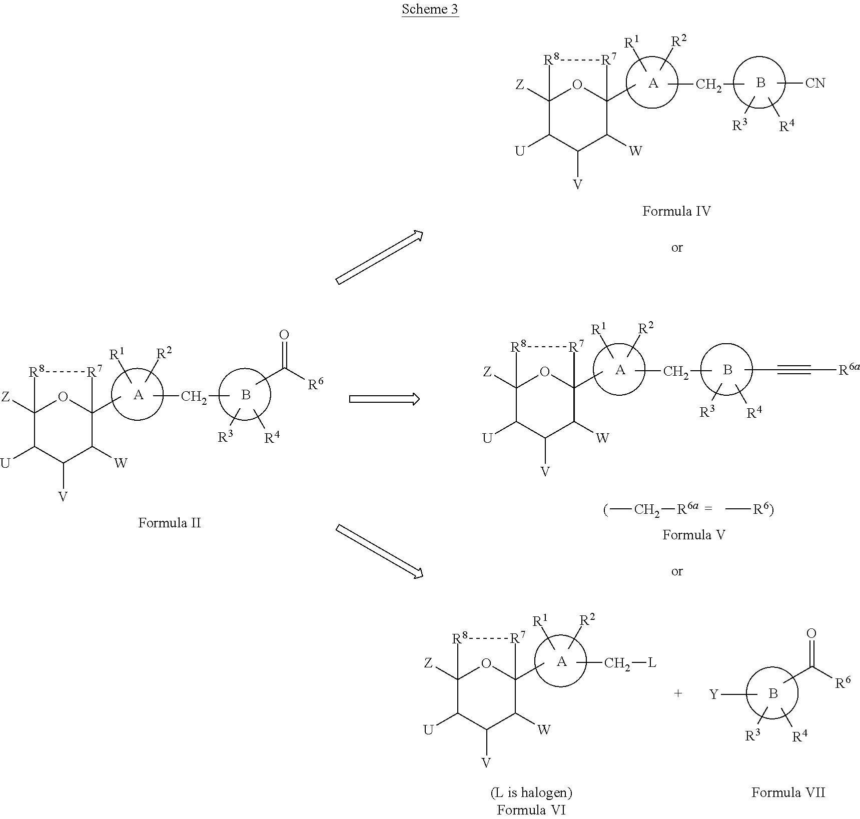

The compounds of the Formula II can be synthesized starting from compounds of Formula IV, Formula V or Formula VI by various routes as shown in Scheme 3.

The compounds of the Formula IV with nitrile functionality can be treated with suitable organometallic reagents such as alkyl/aryl magnesium halides, alkyl/aryl lithium, DIBAL-H and the like in a solvent such as but not limited to diethylether, THF, toluene, DCM or a combination there of to obtain compounds of Formula II. Alternately the treatment of compounds of Formula IV with suitably substituted hydroxylamine derivatives of Formula III (R⁵ONH₂) or its salts can lead to the formation of compounds of the Formula II.

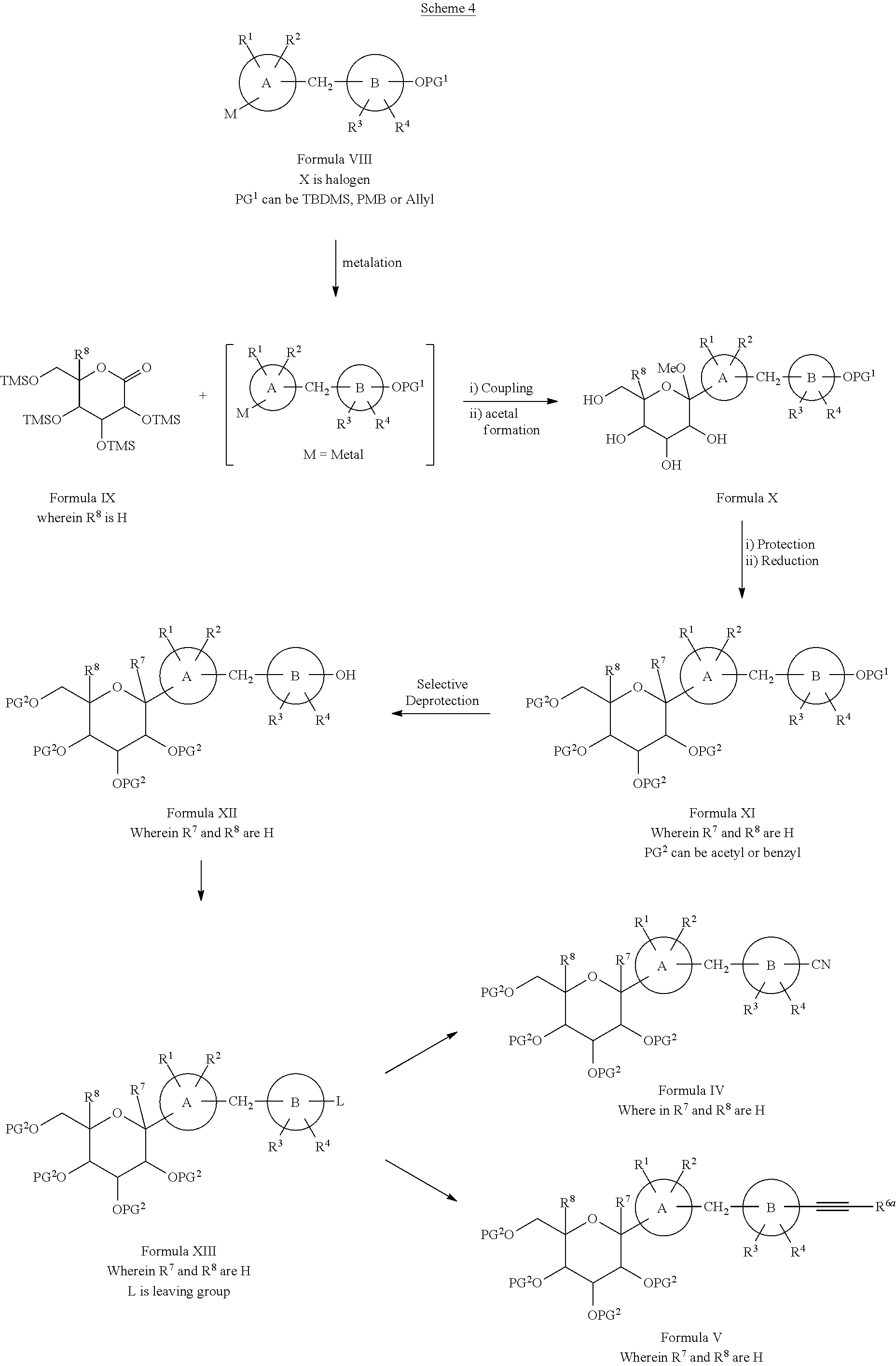

The compounds of the Formula V with alkyne functionality can be hydrated with acids optionally in presence of a suitable catalyst such as but not limited to dil. H₂SO₄, dil. H₂SO₄/HgSO₄ or formic acid to obtain the compounds of the Formula II. Alternately, a sequence of hydroboration and oxidation can be utilized for this transformation with combination of reagents such as Py.BH₃/I₂/H₂O₂/NaOH, bis(1,2-dimethylpropyl)borane/NaBO₃ and the like. Still another way of converting compounds of Formula V to Formula II is metal catalyzed hydration using suitable reagents or combinations such as ⁱPrAuCl/AgSbF₆, Ph₃PAuCl/AgSbF₆, NaAuCl, [(COD)RhCl]₂, [Ru₃(dppm)₃Cl₅]PF₆ and the like.

Alternatively, the compounds of the Formula II can be synthesized by coupling compounds of the Formula VI and Formula VII wherein L represents groups such as but not limited to halogen, triflate or phosphate and Y represents suitably substituted derivatives of Boron, Tin, Zinc, Magnesium or Silicon. The coupling reaction can be conducted in the presence of a metal or its derivatives for example but not limited to Palladium, Nickel, Rhodium, Copper, Iron or Gold.

The compounds of the Formula IV or V can be prepared by following the steps provided in Scheme 4. Compounds of Formula IX and Formula VIII can be prepared by following the procedure given in US20070049537. The obtained compounds of Formula VIII can be metalated with reagents such as but not limited to ⁿBuLi, ^{s}BuLi, ^{t}BuLi, Li, ⁱPrMgCl or ⁱPrMgCl/LiCl in a suitable solvent such as but not limited to toluene, THF, diethyl ether or combination thereof. The resulting metalated species can be treated with Compounds of Formula IX at temperatures ranging from −100 to 50° C. This step is followed by an acetal formation reaction, for example but not limited to treatment with methanesulfonicacid in presence of methanol to obtain compounds of Formula X. The free hydroxyl groups of the compounds of Formula X can protected with acetyl or benzyl groups under the conditions familiar to a person skilled in the art. Such conditions include but not limited to the treatment with acetic anhydride, acetyl chloride, benzyl bromide or benzyl chloride in the presence of a suitable base such as but not limited to NEt₃, DIPEA, Pyridine, DMAP, K₂CO₃ or NaOH in a suitable solvent for example DCM, DMF, THF, acetone or a combination thereof. Thus obtained hydroxy protected compounds of Formula X can be converted to compounds of Formula XI by treating with reagents for example, but not limited to triethylsilane or triisopropylsilane in combination with borontrifluoride etherate, AlCl₃, trifluoroacetic acid, trifluoromethane sulfonic acid in a suitable solvent such as acetonitrile, DCM, THF and the like to furnish the compounds of Formula XI.

The compounds of Formula XI can be converted to the compounds of Formula XII by selective deprotection of PG¹ by the methods known to a person skilled in the art. Such deprotection methods include but not limited to, treatment with acids such as pTSA, trifluoro acetic acid, HCl, H₂SO₄, HBr, HI and the like in a suitable solvent like DCM, dichloroethane, diethylether, diisopropylether, THF, dioxane, actonitrile, methanol and the like or combination thereof. Alternately, compounds of Formula XI can be converted to compounds of Formula XII by deprotection reaction involving reagents such as but not limited to tetrabutylammoniumfluoride or hydrogenfluoride in solvents such as DCM, dichloroethane, THF, dioxane or pyridine and the like. An oxidative deprotection using reagents for example but not limited to DDQ, CAN or a combination of OsO₄, NaIO₄ and NMO in a suitable solvent can also be employed to obtain compounds of Formula XII. Alternately, this selective deprotection can be achieved by metal catalyzed reactions involving Pd(PPh₃)₄ or Pd/C in presence of alkali base or 1,3-dimethyl barbituric acid in a suitable solvent such as but not limited to THF, methanol or DME. The Hydroxy group of the Compounds of Formula XII can be converted to a leaving group such as but not limited to triflate by treating with trifluoromethanesulfonic anhydride in presence of a base for example, but not limited to pyridine or triethylamine in a suitable solvent such as DCM, THF, ACN and the like to furnish compounds of Formula XIII. The compounds of Formula XIII can be converted to compounds of Formula IV by cyanation reaction using a catalyst for example but not limited to Pd₂(dba)₃, Pd(OAc)₂ or PdCl₂ in combination with Zn(CN)₂ or K₄[Fe(CN)₆] and ligand such as but not limited to dppf, PCy₃ or PPh₃ in a suitable solvent; optionally in presence of a suitable base. Alternately, Compounds of Formula XIII can be converted to Formula V by using Sonogashira coupling conditions. This coupling reaction with suitable alkyne can be done by using a catalyst for example but not limited to PdCl₂(PPh₃)₂, Pd(PPh₃)₄, PdCl₂(CH₃CN)₂, PdCl₂, Pd(OAc)₂ or PdCl₂(PCy₃)₂ usually in the presence of CuI and a suitable base such as but not limited to NEt₃, DIPEA, ⁱPr₂NH, DBU, pyrrolidine or Cs₂CO₃ in a suitable solvent such as DMF, NMP, THF or dioxane or a combination thereof.

The compounds of Formula IV or Formula V where in R⁸ and R⁷ are methylene and oxygen respectively which are linked through a bond, can be prepared by following the Scheme 5. Compounds of Formula X can be converted to Compounds of Formula XIV by protecting all free hydroxyl groups with TMS by using TMSCl in the presence of a base such as but not limited to NEt₃, DIPEA, NMM, imidazole or pyridine in a suitable solvent for example THF, DCM or ACN and the like. Thus obtained tetra silyl intermediate can be treated with silyl deprotecting agents for example but not limited to K₂CO₃, TBAF, a combination of KF-crownether, NBS, SiF₄, BF₃.Et₂O, CF₃SO₃SiMe₃ or aqueous acids in a suitable solvent such as MeOH, DCM, THF, ACN and the like, under tuned conditions to achieve selective deprotection of primary hydroxyl group. The compounds of Formula XIV can be oxidized to compounds of Formula XV by using various reagents in combination with DMSO and a base such as but not limited to NEt₃, DIPEA or collidine. The reagents that can be used for this transformation are for example but not limited to Ac₂O, oxalyl chloride, DCC, SO₃.Py. Alternately, the compounds of Formula XIV can be oxidized to compounds of Formula XV by using TPAP, TPAP and NMO, TEMPO, TEMPO and NaOCl, MnO₂, BaMnO₄, CrO₃.Py, PCC, PDC, Dess-Martin periodinane, O-iodoxybenzoic acid, oxone and the like. The compounds of Formula XV can be converted to compounds of Formula XVI by using formaldehyde or paraformaldehyde in the presence of a suitable base such as but not limited to NaOEt, NaOH, KOH, K₂CO₃ or Potassium Phosphate in a suitable solvent such as dioxane, methanol, ethanol, THF or a combination thereof. Alternately the compounds of Formula X can be converted into compounds of Formula XVI by selective conversion of primary hydroxyl group in to a leaving group such as but not limited to tosylate, mesylate or iodo by treatment with p-tolenesulfonyl chloride, methane sulfonyl chloride or iodine in combination with a suitable base such as pyridine, lutidine, collidine, Na₂CO₃ or K₂CO₃, optionally in presence of a solvent such as but not limited to DCM, DCE, DMF or DMSO. Thus obtained intermediate is heated at temperatures ranging from 60 to 180° C. in pyridine, lutidine or collidine under Kornblum oxidation conditions to afford the aldehyde intermediate. This aldehyde can be treated with formaldehyde or paraformaldehyde in the presence of a suitable base such as but not limited to NaOEt, NaOH, KOH, K₂CO₃ or Potassium Phosphate in a suitable solvent such as dioxane, methanol, ethanol, THF or a combination thereof to furnish compounds of formula XVI. The compounds of Formula XVI can be treated with a suitable acid such as methanesulfonic acid, trifluoroacetic acid, silica gel impregnated with pTSA, HCl, H₂SO₄ and the like in a suitable solvent for example but not limited to DCM, DCE, MeOH or acetic acid to furnish the compounds of Formula XVII. Thus obtained bicyclic compounds of Formula XVII can be protected with Acetyl or Benzyl groups by using conditions familiar to the person skilled in the art to obtain compounds of Formula XVIII. Such conditions include but not limited to the treatment with acetic anhydride, acetyl chloride, benzyl bromide, benzyl chloride in the presence of a suitable base such as but not limited to NEt₃, DIPEA, Pyridine, DMAP, K₂CO₃ or NaOH in a suitable solvent for example DCM, DMF, THF, acetone or a combination there of. The compounds of Formula XVIII can be converted to the compounds of Formula XIX by selective deprotection of PG¹ by the methods known to a person skilled in the art. These deprotection methods include but not limited to, treatment with acids such as trifluoroacetic acid, HCl, H₂SO₄, HBr, HI and the like in a suitable solvent like DCM, dichloroethane, diethylether, diisopropylether, THF, dioxane, actonitrile, methanol and the like or combination thereof.

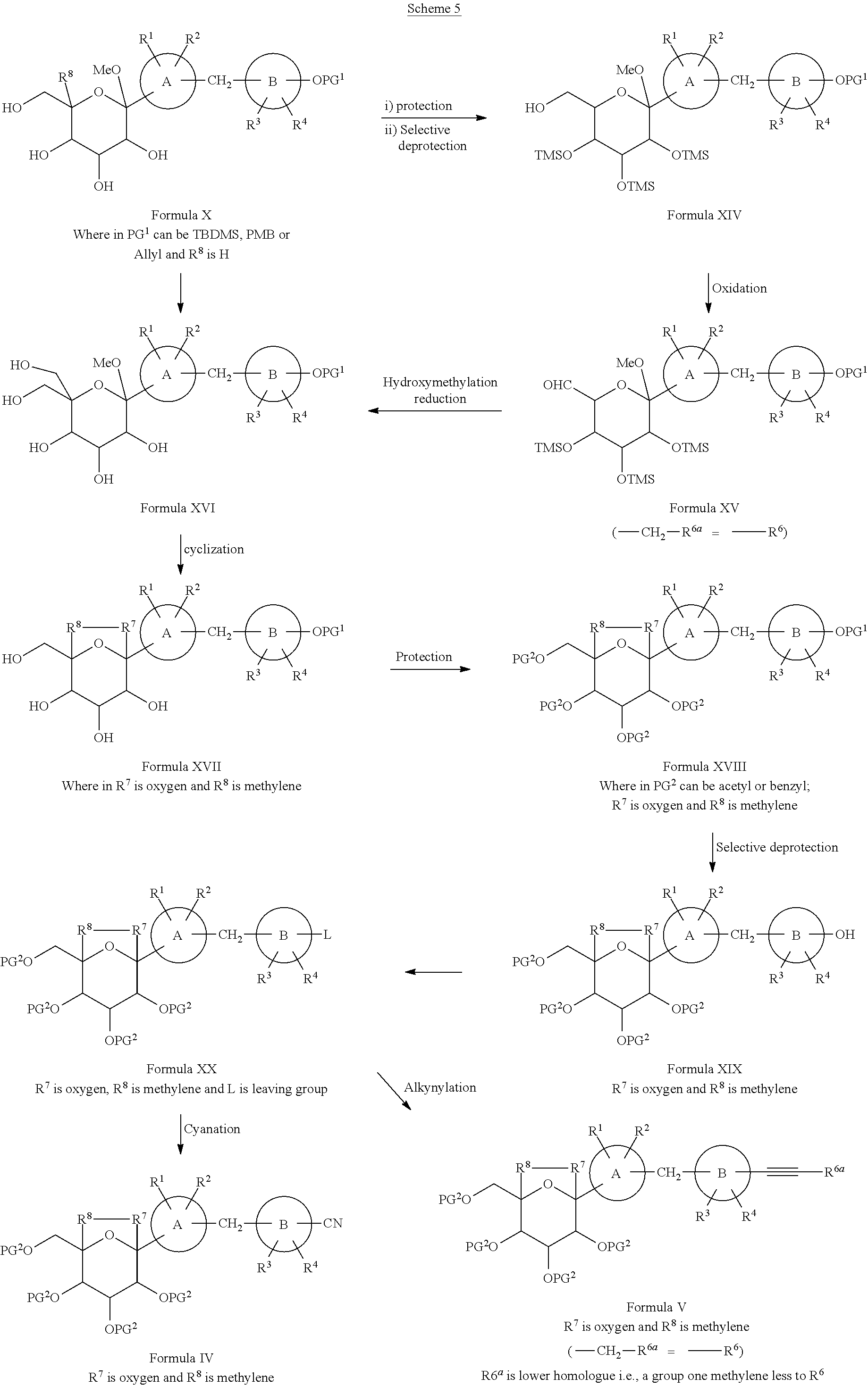

Alternately, compounds of Formula XVIII can be converted to compounds of Formula XIX by deprotection reactions involving reagents such as but not limited to tetrabutylammoniumfluoride or hydrogenfluoride in solvents such as DCM, dichloroethane, THF, dioxane or pyridine and the like. An oxidative method using reagents for example but not limited to DDQ, CAN or a combination of OsO₄, NaIO₄ and NMO in a suitable solvent can also be employed to obtain compounds of Formula XIX. Alternately, this selective deprotection can be achieved by metal catalyzed reactions involving Pd(PPh₃)₄ or Pd/C in presence of alkali base or 1,3-dimethyl barbituric acid in a suitable solvent such as but not limited to THF, methanol or DME. The Hydroxy group of the compounds of Formula XIX can be converted to a leaving group such as but not limited to trifluoromethanesulfonate by treating with trifluoromethanesulfonic anhydride in presence of a base for example, but not limited to pyridine, triethyl amine usually in a suitable solvent such as DCM, THF, ACN and the like to obtain the compounds of Formula XX. The compounds of Formula XX can be converted to compounds of Formula IV by cyanation reaction using a catalyst for example but not limited to Pd₂(dba)₃, Pd(OAc)₂ or PdCl₂ in combination with Zn(CN)₂ or K₄[Fe(CN)₆] and ligand such as but not limited to dppf, PCy₃ or PPh₃ in a suitable solvent; optionally in presence of a suitable base. Alternately, Compounds of Formula XX can be converted to Formula V by using Sonogashira coupling conditions. This coupling reaction with suitable alkyne can be done by using a catalyst for example but not limited to PdCl₂(PPh₃)₂, Pd(PPh₃)₄, PdCl₂(CH₃CN)₂, PdCl₂, Pd(OAc)₂ or PdCl₂(PCy₃)₂ usually in the presence of CuI and a suitable base such as but not limited to NEt₃, DIPEA, ⁱPr₂NH, DBU, pyrrolidine or Cs₂CO₃ in a suitable solvent such as DMF, NMP, THF or dioxane or a combination thereof.

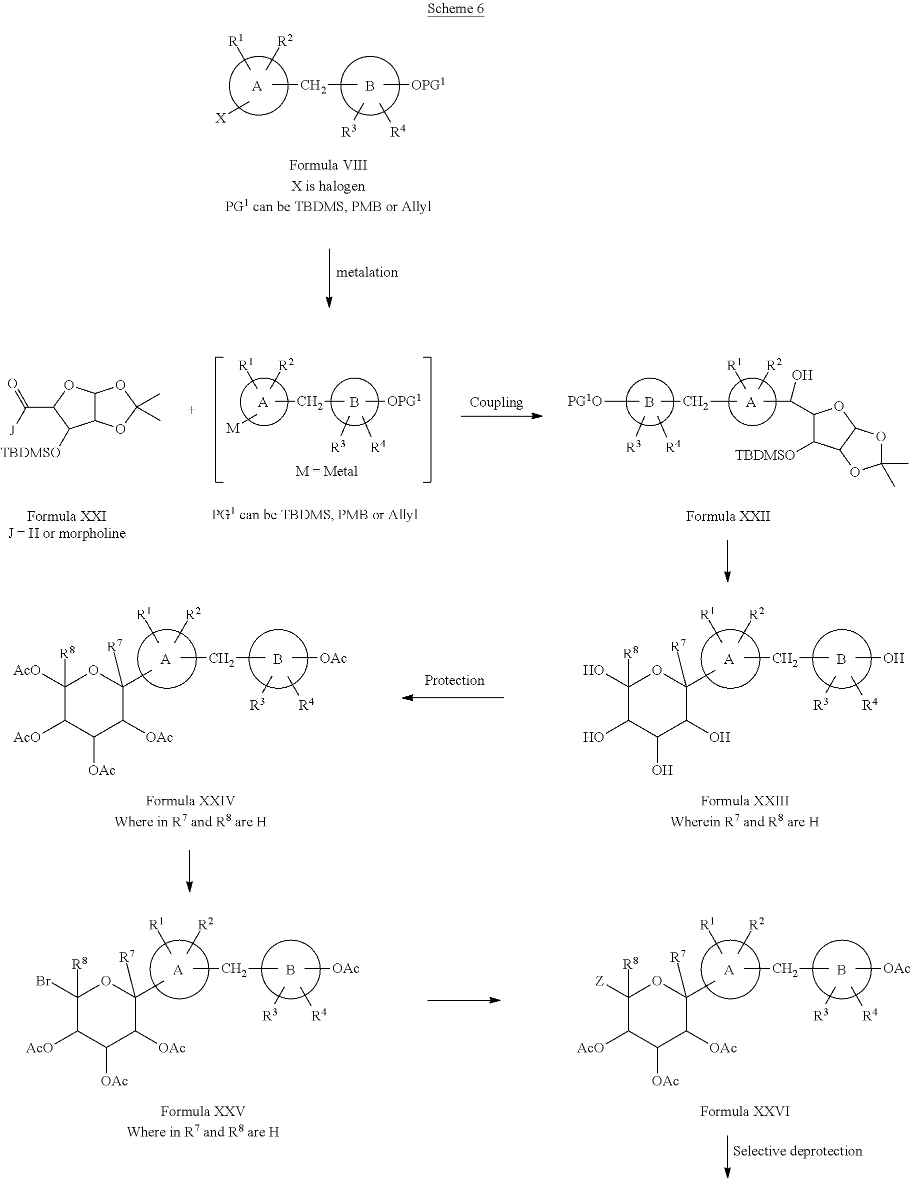

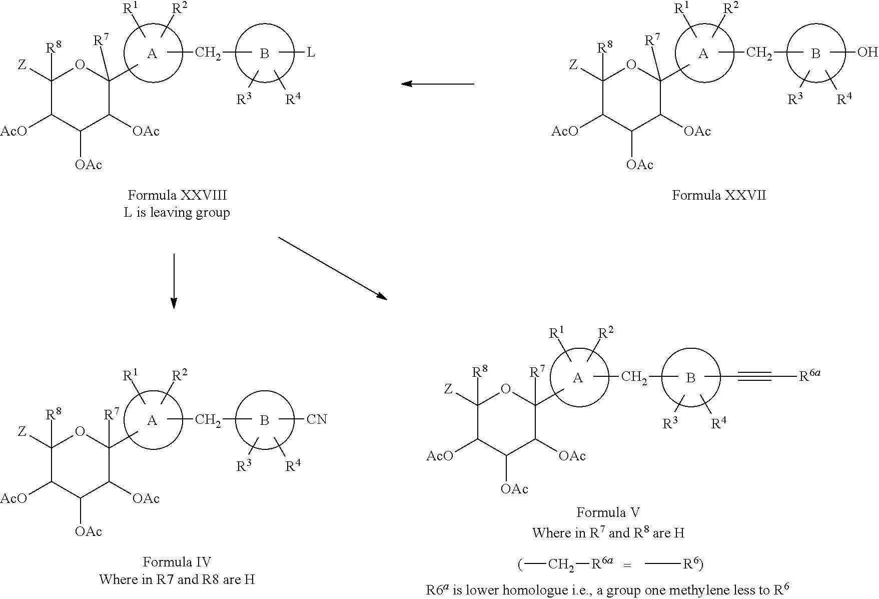

The compounds of the Formula IV and Formula V can also be prepared from compounds of Formula VIII and compounds of Formula XXI by following Scheme 6. The compounds of Formula VIII (can be prepared according to the procedure given in US20070049537) can be metalated by treating with reagents such as but not limited to ⁿBuLi, ^{s}BuLi, ^{t}BuLi, Li, ⁱPrMgCl or ⁱPrMgCl/LiCl in a suitable solvent such as but not limited to toluene, THF, diiethylether or combination thereof. The resulting metalated species can be treated with compounds of Formula XXI (can be prepared by following the procedure given in Nucleoside, Nucloetides & nucleic acids, 2001, 20(4-7), 649-652 or PCT application WO2009014970) at temperatures ranging from −100 to 50° C. to furnish the compounds of Formula XXII. The compounds of Formula XXII thus obtained can be converted to the compounds of Formula XXIII by deprotection methods known to a person skilled in the art. These deprotection methods include but not limited to, treatment with acids such as trifluoroacetic acid, HCl, H₂SO₄, HBr, HI etc. in a suitable solvent like DCM, dichloroethane, diethylether, diisopropylether, THF, dioxane, acetonitrile etc. or combination thereof. The compounds of Formula XXIII can be protected with acetyl groups to obtain compounds of Formula XXIV by treating with acetic anhydride or acetyl chloride in the presence of a suitable base such as but not limited to NEt₃, DIPEA, Pyridine, DMAP, K₂CO₃ or NaOH in a suitable solvent for example but not limited to DCM, DMF, THF, ACN or a combination there of. The compounds of Formula XXIV can be treated with HBr in acetic acid in the presence of a solvent such as but not limited to DCM, DCE or acetic acid to obtain compounds of Formula XXV. The compounds of Formula XXV can be treated with nucleophilic reagents such as but not limited to methanol, ethanol, methyl amine, ethyl amine, methanethiol or ethanethiol in presence of ZnO to obtain compounds of Formula XXVI. Selective deprotection of compounds of Formula XXVI can be achieved by using 1,1,3,3,-tetramethyl guanidine in ACN to furnish compounds of Formula XXVII (as described in Organic Letters 2003, 5, 209). The hydroxy group of the compounds of Formula XXVII can be converted to a leaving group such as but not limited to trifluormethanesulfonate by treating with trifluoromethanesulfonic anhydride in presence of a base for example, but not limited to pyridine, triethyl amine usually in a suitable solvent such as DCM, THF, ACN and the like to obtain the compounds of Formula XXVIII. Compounds of Formula XXVIII can be converted to Formula IV by cyanation reaction using a catalyst for example but not limited to Pd₂(dba)₃, Pd(OAc)₂ or PdCl₂ in combination with Zn(CN)₂ or K₄[Fe(CN)₆] and ligand such as but not limited to dppf, PCy₃ or PPh₃ in a suitable solvent; optionally in presence of a suitable base. Alternately, Compounds of Formula XXVIII can be converted to Formula V by using Sonogashira coupling conditions. This coupling reaction with a suitable alkyne can be done by using a catalyst for example but not limited to PdCl₂(PPh₃)₂, Pd(PPh₃)₄, PdCl₂(CH₃CN)₂, PdCl₂, Pd(OAc)₂ or PdCl₂(PCy₃)₂ usually in the presence of CuI and a suitable base such as but not limited to NEt₃, DIPEA, ⁱPr₂NH, DBU, Pyrrolidine or Cs₂CO₃ in a suitable solvent such as DMF, NMP, THF or dioxane or a combination thereof.

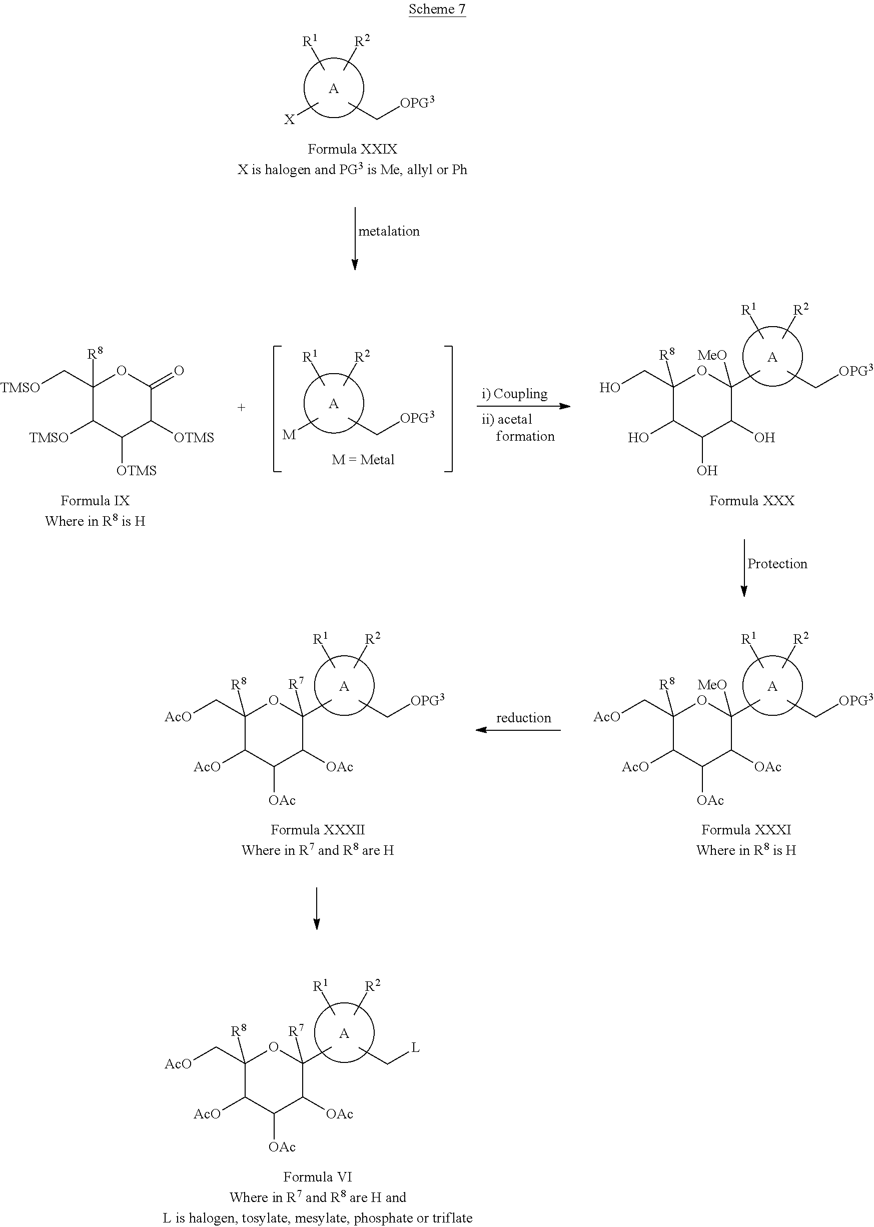

The compounds of the Formula VI can be prepared from compounds of Formula IX and compounds of Formula XXIX by following Scheme 7. The compounds of Formula XXIX (can be prepared according to the procedure given in PCT application WO2008034859) can be metalated by treating with reagents such as but not limited to ⁿBuLi, ^{s}BuLi, ^{t}BuLi, Li, ⁱPrMgCl or ⁱPrMgCl/LiCl in a suitable solvent such as but not limited to toluene, THF, diethylether or combination thereof. The resulting metalated species can be treated with compounds of Formula IX (can be prepared by following the procedure given in US20070049537) at temperatures ranging from −100 to 50° C. In situ treatment of the resulting intermediate with methanesulfonicacid in presence of methanol can lead to the compounds of Formula XXX. The free hydroxyl groups of compounds of Formula XXX can be protected with acetyl groups to obtain compounds of Formula XXXI by treating with acetic anhydride or acetyl chloride in the presence of a suitable base such as but not limited to NEt₃, DIPEA, pyridine, DMAP, K₂CO₃ or NaOH in a suitable solvent for example but not limited to DCM, DMF, THF, ACN or a combination there of. Thus obtained compounds of Formula XXXI can be converted to compounds of Formula XXXII by treating with reagents for example, but not limited to triethylsilane or triisopropylsilane in combination with borontrifluoride etherate, AlCl₃, trifluoroacetic acid, trifluoromethane sulfonic acid in a suitable solvent such as acetonitrile, DCM, THF and the like. The compounds of Formula XXXII upon treatment with acids for example but not limited to HBr, HI, BBr₃ or AlCl₃ in a suitable solvent such as DCM, DCE, Chloroform, Acetic acid and the like can furnish compounds of Formula VI. Alternately, the compounds of Formula XXXII can be subjected to deprotection of OPG³ and the resulting free hydroxyl group can be converted to a leaving group to furnish compounds of Formula VI. Such deprotection conditions include oxidative deprotection using reagents for example but not limited to DDQ, CAN or a combination of OsO₄, NaIO₄ and NMO in a suitable solvent. Alternately, deprotection can also be done by metal catalyzed reactions involving Pd(PPh₃)₄ or Pd/C in presence of alkali base or 1,3-dimethyl barbituric acid in a suitable solvent such as but not limited to THF, methanol or DME. The free hydroxy group of thus obtained intermediate can be converted to a leaving group such as but not limited to triflate, tosylate, mesylate, halogen or phosphate by treating with trifluoromethanesulfonic anhydride, p-toluenesulfonylchloride, methanesulfonyl chloride or P(OEt)₂OCl, PBr₃, SOCl₂ or a combination of CBr₄/PPh₃ optionally in presence of a suitable base for example, but not limited to pyridine, NMM, DIPEA or triethyl amine in a suitable solvent such as DCM, THF, ACN and the like to furnish the compounds of Formula VI.

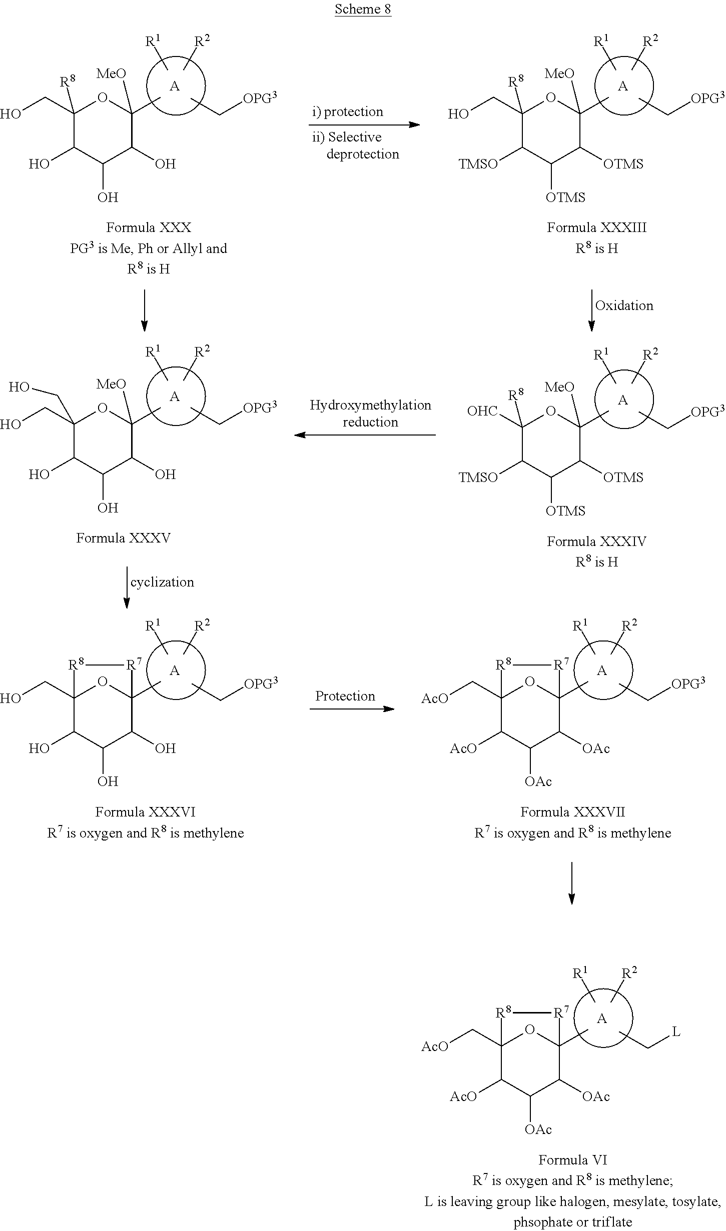

The compounds of the Formula VI where in R⁸ and R⁷ are methylene and oxygen respectively which are linked through a bond, can be prepared from compounds of Formula XXX by following Scheme 8. Compounds of Formula XXX can be converted to compounds of Formula XXXIII by protecting all free hydroxyl groups with TMS by using TMSCl in the presence of a base such as but not limited to NMM, NEt₃, DIPEA, imidazole or pyridine in a suitable solvent for example THF, DCM or ACN and the like. Thus obtained tetra silyl intermediate can be treated with silyl deprotecting agents for example but not limited to K₂CO₃, TBAF, a combination of KF-crownether, NBS, SiF₄, BF₃.Et₂O, CF₃SO₃SiMe₃ or aqueous acids in a suitable solvent such as MeOH, DCM, THF, ACN and the like, under tuned conditions to achieve selective deprotection of primary hydroxyl group. Thus obtained compounds of Formula XXXIII can be oxidized to compounds of Formula XXXIV by using various reagents in combination with DMSO and a base such as but not limited to NEt₃, DIPEA or collidine. The reagents that can be used for this transformation are for example but not limited to Ac₂O, oxalyl chloride, DCC, SO₃.Py. Alternately, the compounds of Formula XXXIII can be oxidized to compounds of Formula XXXIV by using TPAP, TPAP and NMO, TEMPO, TEMPO and NaOCl, MnO₂, BaMnO₄, CrO₃.Py, PCC, PDC, Dess-Martin periodinane, O-iodoxybenzoic acid, oxone and the like. The compounds of Formula XXXIV can be converted to compounds of Formula XXXV by using formaldehyde or paraformaldehyde in the presence of a suitable base such as but not limited to NaOH, KOH, K₂CO₃ or Potassium Phosphate in a suitable solvent such as dioxane, methanol, ethanol, THF or a combination thereof. Alternately the compounds of Formula XXX can be converted into compounds of Formula XXXV by selective conversion of primary hydroxyl group in to a leaving group such as but not limited to tosylate, mesylate or Iodo by treatment with p-tolenesulfonyl chloride, methane sulfonyl chloride or iodine in combination with a suitable base such as pyridine, lutidine, collidine, Na₂CO₃ or K₂CO₃ optionally in presence of a solvent such as but not limited to DCM, DCE, DMF or DMSO. Thus obtained intermediate is heated at temperatures ranging from 60 to 180° C. in pyridine, lutidine or collidine under Kornblum oxidation conditions to afford the aldehyde intermediate. This aldehyde can be treated with formaldehyde or paraformaldehyde in the presence of a suitable base such as but not limited to NaOEt, NaOH, KOH, K₂CO₃ or Potassium Phosphate in a suitable solvent such as dioxane, methanol, ethanol, THF or a combination thereof to furnish compounds of formula XXXV. The compounds of Formula XXXV can be treated with a suitable acid such as methanesulfonic acid, trifluoroacetic acid, HCl, H₂SO₄ and the like in a suitable solvent for example but not limited to DCM, DCE or acetic acid to furnish the compounds of Formula XXXVI. The compounds of Formula XXXVI can be protected with acetyl groups by using acetic anhydride or acetyl chloride in the presence of a suitable base such as but not limited to NEt₃, DIPEA, pyridine, DMAP, K₂CO₃ or NaOH in a suitable solvent for example DCM, DMF, THF, acetone or a combination there of to furnish compounds of Formula XXXVII. The compounds of Formula XXXVII upon treatment with acids for example but not limited to HBr, HI, BBr₃ or AlCl₃ in a suitable solvent such as DCM, DCE, chloroform, acetic acid and the like can furnish compounds of Formula VI. Alternately, the compounds of Formula XXXVII can be subjected to deprotection of OPG³ and the resulting free hydroxyl group can be converted to a leaving group to furnish compounds of Formula VI. Such deprotection conditions include oxidative deprotection using reagents for example but not limited to DDQ, CAN or a combination of OsO₄, NaIO₄ and NMO in a suitable solvent. Alternately, deprotection can also be done by metal catalyzed reactions involving Pd(PPh₃)₄ or Pd/C in presence of alkali base or 1,3-dimethyl barbituric acid in a suitable solvent such as but not limited to THF, methanol or DME. The free hydroxy group of thus obtained intermediate can be converted to a leaving group such as but not limited to triflate, tosylate, mesylate, halogen or phosphate by treating with trifluoromethanesulfonic anhydride, p-toluenesulfonylchloride, methanesulfonyl chloride or P(OEt)₂OCl, PBr₃, SOCl₂ or a combination of CBr₄/PPh₃ optionally in presence of a suitable base for example, but not limited to pyridine, NMM, DIPEA or triethyl amine in a suitable solvent such as DCM, THF, ACN and the like to furnish the compounds of Formula VI.

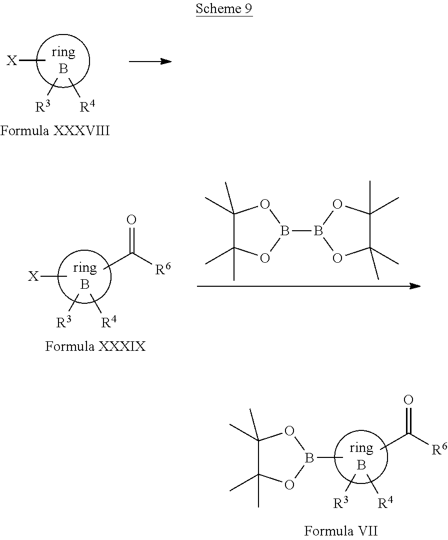

The commercially non available compounds of Formula VII can be prepared by following Scheme 9. The compound of Formula XXXVIII where in X is halogen can be treated with carboxylic acid anhydrides or halides for example but not limited to propionic anhydride, butanoic anhydride, propionyl chloride, butyryl chloride, isobutyryl chloride or isobutyryl chloride and the like in the presence of a Lewis acid such as but not limited to AlCl₃, SnCl₄, BF₃.Et₂O or TiCl₄ to furnish compounds of Formula XXXIX. Thus obtained compounds of Formula XXXIX can be reacted with bis-pinacolatodiborane in the presence of a catalyst such as Pd(dppf)Cl₂, Pd₂(dba)₃, Pd(OAc)₂, Pd(PPh₃)₄ or PdCl₂ in presence of KOAc, NaOAc or a phosphate buffer in a suitable solvent such as but not limited to dioxane, THF, toluene or DCE to obtain compounds of Formula VII.

It is understood that, as used herein, references to the compounds of structural Formula I are meant to also include the pharmaceutically acceptable salts, and also salts that are not pharmaceutically acceptable when they are used as precursors to the free compounds or their pharmaceutically acceptable salts or in other synthetic manipulations. The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. The salts may be prepared during the final isolation and purification of the compounds or separately by making basic or acidic addition salts. Representative salts of basic compounds of the present invention can be prepared by reacting free base form of the compound with a suitable acid, including, but not limited to acetate, trifluoroacetate, adipate, citrate, aspartate, benzoate, benzenesulphonate, bisulfate, besylate, butyrate, camphorsulphonate, difluconate, hemisulfate, heptanoate, formate, fumarate, lactate, maleate, methanesulfonate, naphthyl-sulfonate, nicotinate, oxalate, picrate, pivalate, succinate, tartrate, tirchloracetat, glutamate, p-toluenesulphonate, hydrochloric, hydrobromic, sulphuric, phosphoric and the like. Representative salts of acidic compounds of the present invention can be prepared by reacting free acid form of the compound with a suitable base, including, but not limited to ammonium, calcium, magnesium, potassium, sodium salts, salts of primary, secondary and tertiary amines, substituted amines including naturally occurring ones e.g., arginine, betaine, caffeine, choline, glucamine, glucosamine, histidine, lysine, morpholine, piperazine, piperidine, purine, triethylamine and the like. Compounds of the present invention that contain a carboxylic acid (—COOH) or alcohol group, their pharmaceutically acceptable esters of carboxylic acids such as methyl, ethyl and the like, or acyl derivatives of alcohols such as acetate and the like, can be employed. Compounds of the present invention that comprise basic nitrogen atom may be quaternized with alkyl halides, alkyl sulfates and the like. Such salts permit the preparation of both water soluble and oil soluble compounds of the present invention. It should be recognized that the free base or free acid forms will typically differ from their respective salt forms somewhat in physical properties such as solubility in polar solvents, but otherwise the salts are equivalent to their respective free forms for the purpose of the invention.

The “pharmaceutically acceptable solvates” refer to solvates with water (i.e., hydrates) or pharmaceutically acceptable solvents, for example, ethanol and the like.

The invention also encompasses “prodrugs” of the compounds of the present invention which upon in-vivo administration undergo cleavage by metabolic processes before becoming active pharmacological substances. In general such prodrugs are derivatives of functional group of a compound of the invention which are readily convertible in vivo into the compound of the invention. Conventional procedures for the selection and preparation of suitable prodrug derivatives are described, for example, in “Targeted prodrug design to optimize drug delivery”, AAPS PharmaSci (2000), 2(1), E6. In certain cases, the prodrug itself can also have biological activity in the disease area.

Preferably, the invention encompasses Oxygen prodrugs (O-prodrugs) of the compounds of the present invention which upon in-vivo administration undergo chemical conversion by metabolic processes before becoming active pharmacological substances. In general such O-prodrugs can be represented in general by O-alkyl ethers (methyl, ethyl, substituted alkyl ethers like methoxymethyl, ethoxyethyl and the like), O-allyl ethers, O-benzyl ethers, O-substituted benzyl ethers, O-esters (e.g., formate, benzoyl, acetate, benzoate and the like), or carbonates (e.g., methyl, methoxymethyl and the like) and the like.

The invention also encompasses active “metabolites” of the compound of the present invention. An active metabolite is an active derivative of a SGLT-2 inhibitor produced when the SGLT-2 inhibitor is metabolized.

The present invention includes all pharmaceutically acceptable isotopically-labeled compounds of Formula I wherein one or more atoms are replaced by atoms having the same atomic number, but an atomic mass or mass number different from the atomic mass or mass number usually found in nature. Examples of isotopes suitable for inclusion in the compounds of the present invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Isotopically-labeled compounds of Formula I can generally be prepared by conventional techniques known to those skilled in the art or by processes analogous to those described in the accompanying examples and schemes using an appropriate isotopically-labeled reagent in place of the non-labeled reagent previously employed.

The present invention also includes all the intermediate complexes of the compounds of Formula I, which are active by themselves or can be readily converted to compounds having inhibitory effect on sodium-dependent glucose cotransporter (SGLT), preferably SGLT-2.

The present invention also provides pharmaceutical compositions, comprising compounds of general Formula I or their pharmaceutically acceptable analogs, derivatives, tautomeric forms, isomers, polymorphs, prodrugs, metabolites, salts or solvates thereof together with one or more pharmaceutically acceptable carriers comprising excipients and auxiliaries, which facilitate processing of the active compound into preparations, which can be used pharmaceutically. The pharmaceutical compositions may be in the forms normally employed, such as tablets, capsules, powders, syrups, solutions, suspensions, emulsions, pills, granules, suppositories, pellets, depot formulations and the like, may contain flavourants, sweeteners etc in suitable solid or liquid carriers or diluents, or in suitable sterile media to form injectable solutions or suspensions. Such compositions typically contain from 0.1 to 99.9% by weight of active compound, the remainder of the composition being pharmaceutically acceptable carriers, diluents or solvents.

The pharmaceutical compositions of the present invention can be manufactured by the processes well known in the art, for example, by means of conventional mixing, dissolving, dry granulation, wet granulation, dragee-making, levigating, emulsifying, encapsulating, entrapping, lyophilizing processes or spray drying. The compounds or the pharmaceutical compositions comprising such compounds of the present invention may be administered in the form of any pharmaceutical formulation. The pharmaceutical formulation will depend upon the nature of the active compound and its route of administration. Any route of administration may be used, for example oral, buccal, pulmonary, topical, parenteral (including subcutaneous, intramuscular and intravenous), transdermal, ocular (ophthalmic), by inhalation, intranasal, transmucosal, implant or rectal administration. Preferably the compounds of the present invention are administered orally, parenterally or topically.

An embodiment of the present invention provides a therapeutically effective amount of a compound of Formula I, or its pharmaceutically acceptable derivatives, tautomeric forms, stereoisomers, polymorphs, prodrugs, metabolites, salts or solvates thereof, for use as a pharmaceutical composition.

In another embodiment, the amount of the novel compounds having the Formula I according to the present invention to be incorporated into the pharmaceutical compositions of the present invention can vary over a wide range depending on known factors such as, for example, the disorder to be treated, the severity of the disorder, the patient's body weight, the dosage form, the chosen route of administration and the number of administration per day. Typically, the amount of the compound of Formula I in the pharmaceutical compositions of the present invention will range from approximately 0.01 mg to about 5000 mg. In an embodiment, the daily dose of composition comprising the novel compounds having the Formula I is in the range of about 0.01 mg/kg to about 100 mg/kg based on the body weight of the subject in need thereof which may be administered as a single or multiple doses.

In an embodiment, the novel compounds having the Formula I according to the present invention are particularly useful for the treatment of disease(s) or disorder(s), which are chronic or acute in nature, which favorably respond to or are alleviated by the novel compounds having the Formula I or compositions comprising them. The compositions comprising the novel compounds having the Formula I are useful prophylactically or therapeutically depending upon the pathological condition intended to be prevented or treated respectively.

In one embodiment compounds of the present invention are useful in the prophylaxis, amelioration and/or treatment of one or more condition(s)/disease(s)/disorder(s), in a subject in need thereof.

In another embodiment compounds of the present invention are useful in the prophylaxis, amelioration and/or treatment of one or more condition(s)/disease(s)/disorder(s), which may be regulated or normalized via inhibition of Sodium Glucose Cotransporters (SGLT).

The compounds of the present invention possess activity as selective inhibitors of SGLT-2 and are therefore useful for the prophylaxis, amelioration and/or treatment of variety of diseases, disorders and conditions, including, but not limited to, diabetes (including Type I and Type II), Metabolic Syndrome or ‘Syndrome X’ including impaired glucose tolerance, insulin resistance, metabolic acidosis or ketosis, disorders of food intake, satiety disorders, obesity, hyperinsulinemia, dyslipidemia (including hyperlipidemia, hypertriglyceridemia, hypercholesterolemia, low HDL levels, high LDL levels), hypertension associated with metabolic disorders, congestive heart failure, edema, hyperuricemia, gout, wound healing and tissue ischemia.

The compounds of the present invention can also be used for the prophylaxis, amelioration and/or treatment of the diseases, disorders and conditions collectively referenced to as “diabetic complications” which include both acute complications and chronic complications. Examples of “acute complications” include hyperglycemia (e.g., ketoacidosis), infections (e.g., skin, soft tissue, biliary system, respiratory system and urinary tract infections), etc. Examples of “chronic complications” include microangiopathy (e.g., nephropathy, retinopathy), arteriosclerosis (e.g., atherosclerosis, heart infarction, brian infarction, lower extremity arterial occlusion), neuropathy (e.g, sensory nerves, motor nerves, autonomic nerves), foot gangrene, etc. Major diabetic complications include diabetic retinopathy, diabetic nephropathy and diabetic neuropathy.

All of the various forms and sub-forms of the disorders mentioned herein are contemplated as part of the present invention.

A further embodiment of the present invention is the use of a compound of Formula I for the manufacture of a medicament for the prophylaxis, amelioration and/or treatment of one or more condition(s)/disease(s)/disorder(s) involving SGLT-2 inhibition in a subject in need thereof.

Another embodiment of the present invention is the compound of Formula I or pharmaceutically acceptable derivatives, tautomeric forms, stereoisomers, polymorphs, prodrugs, metabolites, salts or solvates thereof, for use as a medicament.

In still another embodiment of the present invention is the compound of Formula I for use as a medicament for the prophylaxis, amelioration and/or treatment of one or more condition(s)/disease(s)/disorder(s) involving SGLT-2 inhibition, in a subject in need thereof preferably a mammal including a human.

Another embodiment of the present invention provides methods for the prophylaxis, amelioration and/or treatment of one or more condition(s)/disease(s)/disorder(s) involving SGLT-2 inhibition in a subject in need thereof that comprises administering a therapeutically effective amount of compound of Formula I.

In still another embodiment of the present invention is provided use of the dosage form compositions comprising the novel compounds of Formula I for the treatment of one or more condition(s)/disease(s)/disorder(s) involving SGLT-2 inhibition which comprises administrating to a subject in need thereof a pharmaceutically effective amount of the composition.

An embodiment of the present invention relates to methods of using the compounds of Formula I of the present invention or compositions comprising the compounds of Formula I for the prophylaxis, amelioration and/or treatment of any one or more condition(s)/disease(s)/disorder(s) involving SGLT-2 inhibition, which comprises administering to a subject in need thereof the compounds of Formula I or compositions comprising a pharmaceutically effective amount of the compounds of Formula I.

Another embodiment of the present invention provides a therapeutically effective amount of compound of Formula I, or pharmaceutically acceptable derivatives, tautomeric forms, stereoisomers, polymorphs, prodrugs, metabolites, salts or solvates thereof, for use in prophylaxis, amelioration and/or treatment of one or more condition(s)/disease(s)/disorder(s) involving SGLT-2 inhibition, in a subject in need thereof.

In yet another embodiment, the compounds or their pharmaceutically acceptable salts according to the present invention are useful in the treatment of the aforementioned diseases, disorders and conditions in combination with at least one other therapeutic agent. The compounds of the present invention may be used in combination with one or more other therapeutic agents in the treatment, prevention, suppression or amelioration of diseases or conditions for which compounds of the present invention or other therapeutic agents may have utility, where the combination of drugs together are safer or more effective than either drug alone.

Other therapeutic agents suitable for combination with the compounds of the present invention include, but are not limited to, known therapeutic agents useful in the treatment of the aforementioned disorders including: anti-diabetic agents; agents for prevention of complications of diabetes; anti-hyperglycemic agents; hypolipidemic/lipid lowering agents; anti-obesity agents; anti-hypertensive agents, anti-platelet agents, anti-atherosclerotic agents, anti-inflammatory agents, uricosuric agents, anti-TNF agent or c-AMP raising agents and appetite suppressants.

It is believed that the use of the compounds of the present invention in combination with at least one or more of the aforementioned other therapeutic agents may provide results greater than that possible from each of these medicaments alone or greater than the combined additive effects produced by these medicaments. The present compounds and the other therapeutic agents may be administered in the same dosage form or in a separate dosage form by same or different administration route, in dosages and regimens as generally known in the art. Those agents which potentiate the therapeutic effect of SGLT-2 inhibitors according to the invention may allow the dosage to be reduced.

Examples of suitable anti-diabetic agents for use in combination with the compounds of the present invention include but are not limited to (a) other SGLT inhibitors; (b) insulin sensitizers including (i) PPAR γ agonists such as thiozolidinediones or glitazones (e.g. pioglitazone, rosiglitazone and the like), PPAR δ agonists, PPAR α agonists such as fenofibric acid derivatives (gemfibrozil, clofibrate, fenofibrate and bezafibrate), PPARpan agonists, PPAR γ/δ agonists, PPAR α/γ dual agonists, PPAR α/δ dual agonists, PPAR γ antagonists, PPAR α/γ modulators and PPAR α/γ/δ modulators, (ii) biguanides such as metformin and phenformin, and (iii) protein tyrosine phosphatase-1B (PTP-1B) inhibitors; (c) insulin or insulin mimetics; (d) sulfonylureas and other insulin secretagogues, such as tolbutamide, chlorpropamide, tolazamide, glyburide (glibenclamide), glipizide, gliclazide, gliquidone, glimepiride, and meglitinides, such as repaglinide, mitiglinide, nateglinde and the like; (e) glucose absorption inhibitors like alpha.-glucosidase inhibitors (such as acarbose, voglibose and miglitol); (f) glucagon receptor antagonists; (g) GLP-1, GLP-1 mimetics such as exendin-4 or amylin and GLP-1 receptor agonists (h) GIP and GIP mimetics (i) PACAP, PACAP mimetics, and PACAP receptor agonists; (j) AMPK activators; (k) 11β-HSD inhibitors; (1) DPP-IV inhibitors such as Sitagliptin(Merck), Vildag-liptin (Novartis); (m) inhibitors of glucose-6-phosphate, fructose-1,6-biphosphate, glycogen phosphorylase, phosphoenol pyruvate carboxykinase, glycogen synthase kinase, aminopeptidase-N or pyruvate dehydrokinase; (n) glucokinase activators (GKAs); (o) RXR modulators; (p) GPR40 agonists/antagonists, GPR119 agonists or GPR120 agonists; (q) alpha2-antagonists; (r) IBAT inhibitors, HM74a/HM74 agonists, glucocorticoid antagonists, amylin receptor agonists, peptide YY hormone, PEPCK inhibitor, somatotropin release inhibiting factor, CPT-1 inhibitor, insulin receptor kinase stimulants, tripeptidyl peptidase II inhibitors, hepatic gluconeogenesis inhibitors or carboxypeptidase inhibitor.

Examples of suitable agents to be used in combination with the compounds of the present invention, for treatment or prevention of complications of diabetes include but are not limited to GABA-receptor antagonists, Na-channel blockers (e.g. mexiletine hydrochloride, oxacarbazepine or the like), γ-aminobutyric acid receptor antagonists (e.g. topiramat or the like), protein-kinase C inhibitors (e.g. midostaurin or the like), advanced glycation end product inhibitors (e.g. pyridoxamine or the like), transcript factor NF-κB inhibitors (e.g. dexlipotam or the like), lipid peroxide inhibitors (e.g. tirilazad mesylate or the like), α-linked-acid-dipeptidase inhibitors, carnitine derivatives (e.g. levacecamine, levocarnitine or the like), insulin like growth factor-I, platelet-derived growth factor, platelet-derived growth factor analogues, epidermal growth factor, nerve growth factor, biclomol, sulodexide or aldose reductase inhibitors (e.g. ascorbyl gamolenate, tolrestat, epalrestat or the like).

Examples of suitable hypolipidemic/lipid lowering agents for use in combination with the compounds of the present invention include but are not limited to (a) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) bile acid sequestrants (cholestyramine, colestipol, and dialkylaminoalkyl derivatives of a cross-linked dextran), (iii) nicotinyl alcohol, nicotinic acid or a salt thereof, (iv) PPAR agonists as described herein, (v) inhibitors of cholesterol absorption, such as beta-sitosterol and ezetimibe, (vi) acyl CoA cholesterol acyltransferase inhibitors, such as avasimibe, and (vii) anti-oxidants, such as probucol; (b) ileal bile acid transporter inhibitors; (c) HDL raising compounds such as CETP inhibitors or ABC1 regulators (d) lipoxygenase inhibitors; (e) ACAT inhibitors such as avasimibe; (f) fibric acid derivatives i.e. fibrates (e.g. bezafibrate, fenofibrate, gemfibrozil, clofibrate, ciprofibrate, clinofibrate or the like); (g) MTP inhibitors; (h) squalene synthetase inhibitors and squalene epoxidase inhibitors; (i) upregulators of LDL receptor activity; (j) serum cholesterol lowering agents; (k) thyroid hormone receptor agonists (sodium liothyronine, sodium levothyroxine or the like); (l) carnitine palmitoyltransferase inhibitors (etomoxir or the like); (m) probcol and microsomal triglyceride transfer protein inhibitors.

Examples of suitable anti-obesity compounds for use in combination with the compounds of the present invention include but are not limited to (a) fenfluramine, dexfenfluramine, phenteimine, tetrahydrolipostatin, and the like; (b) neuropeptide Y₁ or Y₅ antagonists; (c) CB-1 receptor inverse agonists and antagonists; (d) β₃ adrenergic receptor agonists; (e) melanocortin receptor agonists, in particular melanocortin-4 receptor agonists; (f) ghrelin antagonists; (g) melanin-concentrating hormone (MCH) receptor antagonists; (h) lipase inhibitors like orlistat; (i) serotonin (and dopamine) reuptake inhibitors like sibutramine, topiramate or axokine; (j) thyroid hormone receptor beta drugs; (k) anorectic agents like dexamphetamine, phentermine or mazindol; (l) Leptin analogs.

Examples of suitable appetite suppressants for use in combination with the compounds of the present invention include but are not limited to (a) monoamine reuptake inhibitors; (b) dopamine agonists; (c) leptin analogues; (d) α-melanocyte stimulating hormone; (e) enterostatin agonists; (f) CCK-A agonists; (g) corticotropin releasing hormone; (h) somatostatin; (i) brain-derived neurotrophic factor; (j) orexin receptor agonists.

Examples of suitable anti-hypertensive agents for use in combination with the compounds of the present invention include but are not limited to (a) vasopeptidase inhibitors like Neutral endopeptidase (neprilysin) inhibitors and/or ACE (angiotensin-converting enzyme) inhibitors or dual NEP/ACE inhibitors (enalapril, lisinopril, captopril, quinapril, trandolapril, fosinpril, benazepril, ramipril, enalaprilat, moexipril or perindopril and the like) and/or PKC inhibitors; (b) beta blockers (like metoprolol, propranolol, atenolol, carvedilol or sotalol) and calcium channel blockers (like amlodipine, diltiazem, nifedipine, verapamil or nicardipine); (c) Angiotensin-II receptor blockers (like losartan, candesartan, irbesartan, valsartan, telmisartan or eprosartan); (d) Renin inhibitors e.g., aliskiren; (e) alpha blockers like terazosin, doxazosin or prasozin; (f) diuretics such as hydrochlorothiazide, torasemide, furosemide, spironolactone or indapamide; (g) thrombocyte aggregation inhibitors; (h) endothelin-converting enzyme inhibitors and endothelin receptor antagonists; (i) vasodilating antihypertensive agents e.g. indapamide, todralazine, hydralazine, budralazine or the like; (j) centrally acting antihypertensive agents e.g. reserpine; (k) α₂-adrenoreceptor agonists e.g. clonidine, methyldopa, moxonidine or the like.

Examples of suitable anti-inflammatory agents for use in combination with the compounds of the present invention include but are not limited to aspirin, non-steroidal anti-inflammatory drugs, glucocorticoids, azulfidine, and selective cyclooxygenase-2 inhibitors.

Examples of suitable anti-platelet agents for use in combination with the compounds of the present invention include but are not limited to abciximab, ticlopidine, eptifibatide, dipyridamole, aspirin, anagrelide, tirofiban or clopidogrel.

Examples of suitable agents to be used in combination with the compounds of the present invention, for the treatment or prevention of hyperuricemia or gout include but are not limited to (a) uric acid synthesis inhibitors e.g. allopurinol, oxypurinol or the like; (b) uricosuric agents e.g. benzbromarone, probenecid or the like; (c) urinary alkanizers e.g. sodium hydrogen carbonate, potassium citrate or the like.

### EXAMPLES

The invention is explained in detail in the following examples which are given solely for the purpose of illustration only and therefore should not be construed to limit the scope of the invention. Those skilled in the art will readily understand that known variations of the conditions and processes of the following preparative procedures can be used to prepare the compounds of the present invention.

All solvents used in reactions were freshly distilled. Solvents were dried prior to use wherever necessary by standard methods (Perrin, D. D.; Armarego, W. L. F. Purification of Laboratory Chemicals, Pergamon Press: Oxford, 1988). Mass spectra (MS) were obtained by electron spray ionization (ESI) eV using Applied biosystem 4000 Q TRAP. ¹H NMR were recorded on Bruker 400 MHz Avance II NMR spectrometer in CDCl₃ (until and unless specified). Chemical shifts are reported as δ values in parts per million (ppm), relative to TMS as internal standard. All coupling constant (J) values are given in Hz.

### Abbreviations

The following abbreviations are employed in the examples and elsewhere herein:

| | | | |
|---|---|---|---|
| ¹H NMR | proton nuclear magnetic resonance | ACN | acetonitrile |
| AcOH | acetic acid | Ac₂O | acetic anhydride |
| C. | centigrade | CAN | ceric ammonium nitrate |
| CDCl₃ | deuterated chloroform | CD₃OD | deuterated methanol |
| d | doublet | dd | doublet of doublet |
| DBU | 1,8-Diazabicyclo[5.4.0] undec-7-ene | DCC | N,N′-Dicyclohexyl carbodiimide |
| DCM | dichloromethane | DDQ | dichloro dicyano quinone |
| DIBAL-H | diisobutylaluminium hydride | DIPEA | N,N-Diisopropylethylamine |
| DMAP | 4-Dimethylaminopyridine | DMF | dimethylformamide |
| DMSO | dimethylsulfoxide | dppf | 1,1′-bis(diphenylphosphino) |
| | | | ferrocene |
| ESIMS | electron spray ionization mass | EtOH | ethanol |
| | spectroscopy | | |
| g | gram(s) | h | hour(s) |
| Hz | hertz | ⁱPrMgCl | isopropyl magnesium chloride |
| ⁱPr₂NH | diisopropylamine | J | coupling constant |
| L | litre | LDA | lithium di-isopropyl amide |
| LiHMDS | lithium hexamethyldisilazide | m | multiplet |
| M | molar | MeOH | methanol |
| mesylate | methane sulfonate | mg | milligram |
| MHz | mega hertz | min | minutes |
| mmol | millimoles | mpk | milligram/kilogram |
| NaOAc | sodium acetate | NBS | N-bromosuccinimide |
| ⁿBuLi | n-butyl lithium | nM | nano molar |
| NMM | N-methylmorpholine | NMO | N-methylmorpholine-N-oxide |
| NMP | N-methyl-2-pyrrolidone | NMR | nuclear magnetic resonance |
| OAc | acetoxy | P(OEt)₂OCl | diethoxyphosphoryl chloride |
| PCy₃ | tricyclohexylphosphine | Pd₂(dba)₃ | tris(dibenzylideneacetone) |
| | | | dipalladium(0) |
| Pet. Ether | petroleum ether | PG | protecting group |
| pH | potential hydrogen | PMB | p-methoxybenzyl |
| PPh₃ | triphenylphosphine | pTSA | p-toluenesulfonic acid |
| Py | pyridine | q | quartet |
| r.t. | room temperature | s | singlet |
| t | triplet | TBAF | tetra-n-butylammonium fluoride |
| TBDMS | tert-butyldimethylsilyl | ^{t}BuLi | tert-butyl lithium |
| TEMPO | (2,2,6,6-Tetramethylpiperidin-1-yl)oxyl | TFA | trifluoro acetic acid |
| THF | tetrahydrofuran | TLC | thin layer chromatography |
| TMS | tetramethylsilane | TMSCl | trimethylsilyl chloride |
| tosylate | p-toluenesulfonate | TPAP | tetrapropylammonium perruthenate |
| triflate | trifluoromethanesulfonate | UGE | urinary glucose excretion |
| μg | microgram | | |

### Intermediate 1: Preparation of (2R,3R,4R,5S,6S)-2-(acetoxymethyl)-6-(3-(bromomethyl)-4-chlorophenyl)tetrahydro-2H-pyran-3,4,5-triyl triacetate

### Step 1: Preparation of (5-bromo-2-chlorophenyl) methanol

5-bromo-2-chlorobenzoic acid (20 g, 84.92 mmol) in dry THF (50 mL) was added to a solution of sodium borohydride (8 g, 212.34 mmol) in dry THF (128 mL) in 15 min at r.t. To the resulting reaction mixture a solution of Iodine (26.85 g, 106.15 mmol) in dry THF (170 mL) was added in another 15 min. Stirring was continued till the Iodine color disappeared after which the reaction mixture was heated to reflux temperature for 18 h. After completion of the reaction as confirmed by TLC, volatiles were evaporated in vacuo. The residue obtained was dissolved in ethyl acetate (200 mL), washed with a saturated solution of NaHSO₃ (200 mL) and brine (200 mL) successively. The organic layer was dried over Na₂SO₄ and concentrated in vacuo to afford crude product which was purified by column chromatography (silica gel, 3:7 Ethyl acetate:Pet. Ether) to afford the title compound (18.3 g, 97%).

ESIMS (m/z): 222.8 (M+1)

### Step 2: Preparation of 4-bromo-1-chloro-2-(chloromethyl)benzene

(5-Bromo-2-chlorophenyl) methanol (11.37 g, 51.22 mmol) was taken in aqueous HCl (35%) (1.54 L) and stirred at 70-80° C. for 18 h. After completion of the reaction as confirmed by TLC, the reaction mixture was cooled in an ice bath. The precipitate obtained was collected over Buchner filter, washed with water (2×500 mL) and dried in vacuo to afford the title compound (12.2 g, 99%).

ESIMS (m/z): 240.7 (M+1)

### Step 3: Preparation of 4-bromo-1-chloro-2-(phenoxymethyl)benzene

To a solution of 4-bromo-1-chloro-2-(chloromethyl)benzene (12.2 g, 50.85 mmol) and phenol (4.79 g, 50.85 mmol) in DMF (102 mL) under nitrogen atmosphere, K₂CO₃ was added (35.14 g, 254.25 mmol). The resulting mixture was stirred at r.t. for 18 h. After completion of the reaction as confirmed by TLC, the reaction mixture was passed through celite plug and the filterate was evaporated. The residue obtained was dissolved in ethyl acetate (100 mL) and washed with water (2×100 mL) and brine (2×100 mL) successively. The organic layer was dried over Na₂SO₄ and concentrated in vacuo to afford crude product which was purified by column chromatography (silica gel, 1:9 Ethyl acetate:Pet. Ether) to afford the title compound (14.7 g, 97.3%).

ESIMS (m/z): 297.6 (M−1)

### Step 4: Preparation of (2S,3R,4S,5S,6R)-2-(4-chloro-3-(phenoxymethyl)phenyl)-6-(hydroxymethyl)-2-methoxytetrahydro-2H-pyran-3,4,5-triol

To a solution of 4-bromo-1-chloro-2-(phenoxymethyl)benzene (10.0 g, 33.61 mmol) in dry Toluene (200 mL) and dry THF (100 L), ⁿBuLi (33.61 mL, 53.77 mmol, 1.6 M solution in pentane) was added at −78° C. The reaction mixture was stirred at the same temperature for 15 min A solution of (3R,4S,5R,6R)-3,4,5-tris((trimethylsilyl)oxy)-6-(((trimethylsilyl)oxy)methyl)tetrahydro-2H-pyran-2-one (18.80 g, 40.33 mmol, prepared following the procedure given in US20070049537) in dry toluene (170 mL) was introduced into it at the same temperature and stirred for another 3 h. After completion of the reaction as confirmed by TLC, water (90 mL) was added to at the same temperature and allowed to stir while raising the temperature to r.t. The reaction mixture was diluted with ethyl acetate (200 mL) and the separated aqueous layer was discarded. The organic layer was dried over Na₂SO₄ and concentrated in vacuo. The residue obtained was dissolved in methanol (90 mL) and methanesulfonic acid (2.15 mL, 33.56 mmol) was added at 0° C. The temperature was raised to r.t. gradually and stirred for 16 h. The reaction was quenched by the addition of NEt₃ (14 mL, 50.5 mmol) at 0° C. The volatiles were evaporated in vacuo and the residue obtained was dissolved in ethyl acetate (500 mL), washed with water (200 mL) and brine (200 mL) successively. The organic layer was dried over anhydrous Na₂SO₄ and volatiles were evaporated in vacuo. The residue obtained was purified by column chromatography (silica gel, 1:9 MeOH:DCM) to provide title compound (9.5 g, 68.8%).

ESIMS (m/z): 410.0 (M−1)

### Step 5: Preparation of (2S,3R,4S,5R,6R)-6-(acetoxymethyl)-2-(4-chloro-3-(phenoxymethyl)phenyl)-2-methoxytetrahydro-2H-pyran-3,4,5-triyl triacetate

To a solution of (2S,3R,4S,5S,6R)-2-(4-chloro-3-(phenoxymethyl)phenyl)-6-(hydroxymethyl)-2-methoxytetrahydro-2H-pyran-3,4,5-triol (9.5 g, 23.12 mmol), DIPEA (28.86 ml, 168.80 mmol) and DMAP (1.02 g, 8.32 mmol) in THF (87 mL) was added acetic anhydride (17.73 mL, 187.59 mmol) at 0° C. The reaction was stirred at r.t. for 2 h. After the completion of reaction as confirmed by TLC, the reaction mixture was diluted with ethyl acetate (100 mL) and washed with 10% aqueous HCl (50 mL), saturated NaHCO₃ (50 mL) and water (50 mL) successively. The organic layer was dried over Na₂SO₄ and concentrated in vacuo to afford crude product which was purified by column chromatography (silica gel, 3:7 Ethyl acetate:Pet. Ether) to afford the title compound (12.18 g, 91%).

ESIMS (m/z): 596.2 (M+18)

### Step 6: Preparation of (2R,3R,4R,5S,6S)-2-(acetoxymethyl)-6-(4-chloro-3-(phenoxymethyl)phenyl)tetrahydro-2H-pyran-3,4,5-triyl triacetate

To a solution of (2S,3R,4S,5R,6R)-6-(acetoxymethyl)-2-(4-chloro-3-(phenoxymethyl)phenyl)-2-methoxytetrahydro-2H-pyran-3,4,5-triyl triacetate (12.00 g, 20.73 mmol), triethylsilane (10.46 mL, 65.51 mmol) and water (373 mg, 20.73 mmol) in ACN (62.18 mL) was added BF₃.Et₂O (5.21 mL, 41.46 mmol) at 0° C. The reaction was stirred at r.t. for 18 h. After the completion of reaction as confirmed by TLC, the reaction mixture was diluted with ethyl acetate (100 mL) and washed with saturated NaHCO₃ (50 mL) The organic layer was dried over Na₂SO₄ and concentrated in vacuo to afford crude product which was purified by column chromatography (silica gel, 3:7 Ethyl acetate:Pet. Ether) to afford the title compound (10.58 g, 93%).

ESIMS (m/z): 571.7 (M+23)

### Step 7: Preparation of (2R,3R,4R,5S,6S)-2-(acetoxymethyl)-6-(3-(bromomethyl)-4-chlorophenyl)tetrahydro-2H-pyran-3,4,5-triyl triacetate

To a solution of (2R,3R,4R,5S,6S)-2-(acetoxymethyl)-6-(4-chloro-3-(phenoxymethyl)phenyl)tetrahydro-2H-pyran-3,4,5-triyl triacetate (10.00 g, 18.22 mmol) in acetic acid (31 mL) was added HBr in acetic acid (45.55 mL, 2.5 mL/mmol) and the reaction was stirred at r.t. for 18 h. After the completion of reaction as confirmed by TLC, the reaction mixture was neutralized by the addition of saturated K₂CO₃ at 0° C. and extracted with ethyl acetate (2×50 mL). The combined organic layers were washed with water (50 mL) and brine (50 mL) successively. The organic layer was dried over Na₂SO₄ and concentrated in vacuo to afford crude product which was purified by column chromatography (silica gel, 3:7 Ethyl acetate:Pet. Ether) to afford the title compound (7.9 g, 81%).

ESIMS (m/z): 554.6 (M+18)

### Intermediate 2: Preparation of 1-(4-(4,4,5,5-tetramethyl-1,3,2-dioxaborolan-2-yl)phenyl)propan-1-one

### Step 1: Preparation of 1-(4-bromophenyl)propan-1-one

To a solution of bromobenzene (25 g, 159.2 mmol) and AlCl₃ (25 g, 191 mmol) was added propionyl chloride (14.18 mL, 163.19 mmol) at 50° C. The reaction was stirred at the same temperature for 2 h. After the completion of reaction as confirmed by TLC, the reaction contents were added to ice in a beaker and conc. HCl (50 mL) was added to it. The reaction mixture was extracted with DCM (2×100 mL). The combined organic layers were dried over Na₂SO₄ and concentrated in vacuo to afford crude product which was purified by column chromatography (silica gel, 1:9 Ethyl acetate:Pet. Ether) to afford the title compound (30 g, 88%).

ESIMS (m/z): 230 (M+18)

### Step 2: Preparation of 1-(4-(4,4,5,5-tetramethyl-1,3,2-dioxaborolan-2-yl)phenyl)propan-1-one

To a solution of bispinacolatodiborane (32.76 g, 129.03 mmol), KOAc (34.5 g, 352.1 mmol) and Pd(dppf)Cl₂ (2.87 g, 3.51 mmol) in dioxane (530 mL) was added a solution of 1-(4-bromophenyl)-2-methylpropan-1-one (25 g, 117.3 mmol) in dioxane (50 mL) under N₂ atmosphere. The resulting mixture was stirred at 80° C. for 18 h. After completion of the reaction as confirmed by TLC, volatiles from the reaction mixture were evaporated in vacuo. The residue obtained was diluted with ethyl acetate (500 mL) and washed with water (2×100 mL). The organic layer was dried over Na₂SO₄ and concentrated in vacuo to afford crude product which was purified by column chromatography (silica gel, 1:9 Acetone:Pet. Ether) to afford the title compound (30 g, 98.4%).

ESIMS (m/z): 261.7 (M+1)

### Intermediate 3: Preparation of 1-(4-(4,4,5,5-tetramethyl-1,3,2-dioxaborolan-2-yl)phenyl)ethanone

To a solution of 4-bromoacetophenone (10.0 g, 50.23 mmol) in 1,4-dioxane (150 mL) under nitrogen atmosphere, was added bis(pinacolato)diboron (15.30 g, 60.27 mmol), Pd(dppf)Cl₂.CH₂Cl₂ (1.23 g, 1.520 mmol) and potassium acetate (14.79 g, 150.68 mmol). The reaction mixture was heated to 90° C. for 12 h. After the completion of the reaction as confirmed by TLC, the solution was cooled to r.t. and filtered through celite. The solvent was concentrated in vacuo. The crude compound was extracted with ethyl acetate (700 mL). The organic layer was washed with water, separated, dried over Na₂SO₄ and concentrated in vacuo to afford crude compound which was purified by column chromatography (silica gel, 1:9 Ethyl acetate:Pet. Ether) to afford the title compound (11.0 g, 91%) as a white solid.

ESIMS (m/z): 247.2 (M+1)

### Example 1

### Preparation of 1-(4-(2-chloro-5-((2S,3R,4R,5S,6S)-3,4,5-trihydroxy-6-methoxytetrahydro-2H-pyran-2-yl)benzyl)phenyl)ethanone O-methyl oxime

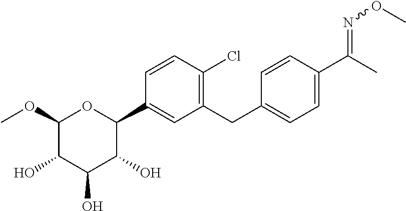

### Step 1: Preparation of ((3aS,6R,6aS)-6-((tert-butyldimethylsilyl)oxy)-2,2-dimethyl tetrahydrofuro[2,3-d][1,3]dioxol-5-yl)(3-(4-((tert-butyldimethylsilyl)oxy)benzyl)-4-chlorophenyl)methanol

A solution of (4-(5-bromo-2-chlorobenzyl)phenoxy)(tert-butyl)dimethylsilane (14.2 g, 34.57 mmol, prepared following the procedure given in US20070049537) in dry THF (90 mL) was cooled to −78° C. and ⁿBuLi (30.7 mL, 46.05 mmol, 1.5 M solution in hexane) was added dropwise while stirring and stirring was continued for further 30 min. A solution of (3aS,5R,6R,6aS)-6-((tert-butyldimethylsilyl)oxy)-2,2-dimethyltetrahydrofuro[2,3-d][1,3]dioxole-5-carbaldehyde (7.0 g, 23.05 mmol, prepared according to the procedure given in Nucleoside, Nucloetides & Nucleic Acids, 2001, 20(4-7), 649-652) in dry THF (13 mL) was added dropwise to the reaction mixture and stirred at the same temperature for 2 h. Reaction temperature was raised slowly to 0° C. and then stirred at r.t. for 16 h. After completion of reaction, as confirmed by TLC, reaction mixture was quenched by the addition of saturated NH₄Cl solution and extracted with ethyl acetate (3×300 mL). The combined organic layers were dried over Na₂SO₄ and the volatiles were evaporated in vacuo. The residue obtained was purified by column chromatography (silica gel, 0.2:9.8 acetone:Pet. Ether) to afford the title compound (3.66 g, 25%).

ESIMS (m/z): 657.0 (M+23)

### Step 2: Preparation of (3S,4R,5S,6S)-6-(3-(4-acetoxybenzyl)-4-chlorophenyl)tetrahydro-2H-pyran-2,3,4,5-tetrayl tetraacetate

A solution of ((3aS,6R,6aS)-6-((tert-butyldimethylsilyl)oxy)-2,2-dimethyl tetrahydrofuro[2,3-d][1,3]dioxol-5-yl)(3-(4-((tert-butyldimethylsilyl)oxy)benzyl)-4-chlorophenyl)methanol (3.66 g, 5.76 mmol) in 3:2 acetic acid and water (30 mL) was refluxed at 110° C. for 22 h. After completion of reaction, as confirmed by TLC, reaction mixture was concentrated in vacuo. Toluene (3×10 mL) was added and distilled and the residue obtained was dissolved in pyridine (15 mL). The resulting mixture was treated with acetic anhydride (4.0 mL, 42.62 mmol) at r.t. for 16 h. After completion of reaction, as confirmed by TLC, water was added and stirred for 1 h. The reaction mixture was diluted with ethylacetate (100 mL) and washed with saturated NaHCO₃ solution (50 mL) and brine (50 mL). The organic layers were dried over Na₂SO₄ and the volatiles were removed in vacuo. The residue obtained was purified by column chromatography (silica gel, 2:8 acetone:Pet. Ether) to afford the title compound (2.03 g, 70%).

ESIMS (m/z): 598.3 (M+23)

### Step 3: Preparation of (3S,4R,5S,6S)-2-bromo-6-(4-chloro-3-(4-hydroxybenzyl) phenyl)tetrahydro-2H-pyran-3,4,5-triyl triacetate

To (3S,4R,5S,6S)-6-(3-(4-acetoxybenzyl)-4-chlorophenyl)tetrahydro-2H-pyran-2,3,4,5-tetrayl tetraacetate (2.03 g, 4.03 mmol) 33% solution of HBr in AcOH (6 mL) was added at r.t. and stirred for 1 h. The reaction mixture was diluted with DCM (20 mL) and stirred for 30 min Water was added to the resulting mixture and stirred for another 1 h, diluted it with DCM (50 mL), washed with water (50 mL), saturated NaHCO₃ solution (50 mL) and brine (50 mL). The organic layer was dried over Na₂SO₄ and the volatiles were evaporated in vacuo to afford the title compound (2.02 g, 90%).

ESIMS (m/z): 554.9 (M+1)

### Step 4: Preparation of (2S,3S,4R,5S,6S)-2-(4-chloro-3-(4-hydroxybenzyl)phenyl)-6-methoxytetrahydro-2H-pyran-3,4,5-triyl triacetate

To a solution of (3S,4R,5S,6S)-2-bromo-6-(4-chloro-3-(4-hydroxybenzyl) phenyl)tetrahydro-2H-pyran-3,4,5-triyl triacetate (2.02 g, 3.62 mmol) in methanol (15 mL) was added ZnO (294.5 mg, 3.62 mmol) at 60° C. The reaction mixture was stirred at 60° C. for 1 h. After completion of reaction, as confirmed by TLC, reaction mixture was passed through sintered funnel to remove the solids. The filerate was evaporated in vacuo and the residue obtained was purified by column chromatography (silica gel, 2:8 acetone:Pet. Ether) to afford title compound (1.1 g, 65%).

ESIMS (m/z): 506.0 (M−1)

### Step 5: Preparation of (2S,3S,4R,5S,6S)-2-(4-chloro-3-(4-(trifluoromethylsulfonyloxy)benzyl)phenyl)-6-methoxytetrahydro-2H-pyran-3,4,5-triyl triacetate

To a solution of (2S,3S,4R,5S,6S)-2-(4-chloro-3-(4-hydroxybenzyl)phenyl)-6-methoxytetrahydro-2H-pyran-3,4,5-triyl triacetate (572 mg, 1.13 mmol) in dry DCM (10 mL), was added NEt₃ (0.63 mL, 4.5 mmol), followed by addition of triflic anhydride (0.37 mL, 2.25 mmol) at 0° C. The reaction was stirred at r.t. for 2 h. After the completion of reaction as observed by TLC, the reaction mixture was diluted with ethyl acetate (100 mL), washed with water (2×30 mL) and dried over Na₂SO₄. The combined organic layers were evaporated in vacuo to obtain the crude compound which was purified by column chromatography (silica gel, 3:7 ethyl acetate:Pet. Ether) to provide the title compound (490 mg, 65.7%).

### Step 6: Preparation of (2S,3S,4R,5S,6S)-2-(4-chloro-3-(4-cyanobenzyl)phenyl)-6-methoxytetrahydro-2H-pyran-3,4,5-triyl triacetate

To a solution of (2S,3S,4R,5S,6S)-2-(4-chloro-3-(4-(trifluoromethylsulfonyloxy)benzyl)phenyl)-6-methoxytetrahydro-2H-pyran-3,4,5-triyl triacetate (490 mg, 0.76 mmol) in dry DMF (10 mL) was added tetrakis(triphenylphosphine)palladium(0) (442.9 mg, 0.38 mmol) followed by addition of zinc cyanide (270 mg, 2.3 mmol) 1,1′-bis(diphenylphosphino)ferrocene (212 mg, 0.38 mmol). The reaction mixture was refluxed at 150° C. for 3 h. After the completion of reaction as observed by TLC, the reaction mixture was cooled to r.t., diluted with ethyl acetate (100 mL), washed with water (2×20 mL) and dried over Na₂SO₄. The combined organic layers were evaporated in vacuo to obtain the crude compound which was purified by column chromatography (silica gel, 3.5:6.5 Ethyl acetate:Pet. Ether) to provide the title compound (230 mg, 58.1%).

ESIMS (m/z): 533.5 (M+18)

### Step 7: Preparation of 4-(2-chloro-5-((2S,3R,4R,5S,6S)-3,4,5-trihydroxy-6-methoxytetrahydro-2H-pyran-2-yl)benzyl)benzonitrile

To a solution of (2S,3S,4R,5S,6S)-2-(4-chloro-3-(4-cyanobenzyl)phenyl)-6-methoxytetrahydro-2H-pyran-3,4,5-triyl triacetate (230 mg, 0.446 mmol) in THF (0.7 mL), methanol (1.2 mL) and water (0.35 mL) was added lithium hydroxide monohydrate (37.5 mg, 0.89 mmol) at 0° C. The reaction was stirred at r.t. for 1 h. volatiles were evaporated in vacuo and the residue obtained was used as such in the next step.

ESIMS (m/z): 407.7 (M+18)

### Step 8: Preparation of 1-(4-(2-chloro-5-((2S,3R,4R,5S,6S)-3,4,5-trihydroxy-6-methoxytetrahydro-2H-pyran-2-yl)benzyl)phenyl)ethanone

To a solution of 4-(2-chloro-5-((2S,3R,4R,5S,6S)-3,4,5-trihydroxy-6-methoxytetrahydro-2H-pyran-2-yl)benzyl)benzonitrile (160 mg, 0.41 mmol) in THF (10 mL) and toluene (10 mL) was added 1.0 M methyl magnesium iodide in ether (5 mL, 5 mmol) at r.t. The reaction mixture was refluxed at 90° C. for 12 h. After the completion of reaction as observed by TLC, the reaction mixture was cooled to r.t., and aqueous ammonium chloride (10 mL) was added. The reaction mixture was diluted with ethyl acetate (50 mL), washed with water (2×20 mL) and dried over Na₂SO₄. The combined organic layers were evaporated in vacuo to obtain the crude compound which was purified by column chromatography (silica gel, 1:9 MeOH:DCM) to provide the title compound (130 mg, 77.8%).

ESIMS (m/z): 407.7 (M+1)

### Step 9: Preparation of 1-(4-(2-chloro-5-((2S,3R,4R,5S,6S)-3,4,5-trihydroxy-6-methoxytetrahydro-2H-pyran-2-yl)benzyl)phenyl)ethanone O-methyl oxime

To a solution of 1-(4-(2-chloro-5-((2S,3R,4R,5S,6S)-3,4,5-trihydroxy-6-methoxytetrahydro-2H-pyran-2-yl)benzyl)phenyl)ethanone (130 mg, 0.319 mmol), in ethanol (10 mL) under nitrogen atmosphere, were added and pyridine (0.128 mL, 1.59 mmol), sodium acetate (261 mg, 3.19 mmol) and O-methylhydroxylamine hydrochloride (132 mg, 1.59 mmol). The reaction mixture was refluxed for 5 h. After completion of the reaction as confirmed by TLC, the solvent was evaporated. The crude compound was extracted with ethyl acetate (3×25 mL). The organic layer was separated, washed with water (3×10 mL), dried over Na₂SO₄ and concentrated in vacuo to afford crude product which was purified by column chromatography (silica gel, 1:9 MeOH:DCM) to afford the title compound (80 mg, 63.8%).

ESIMS (m/z): 436.7 (M+1)

¹H NMR (400 MHz, CD₃OD): δ 2.14-2.17 (s, 3H), 3.26-3.29 (m, 2H), 3.43 (m, 1H), 3.47 (s, 3H), 3.92 (s, 3H), 4.07-4.16 (m, 3H), 4.29 (d, J=7.7 Hz, 1H), 7.19-7.21 (m, 2H), 7.27 (dd, J=8.1 and 2.1 Hz, 1H), 7.30-7.31 (m, 1H), 7.37 (d, J=8.2 Hz, 1H), 7.54-7.56 (m, 2H)

### Example 2

### Preparation of 1-(4-(2-chloro-5-((2S,3R,4R,5S,6R)-3,4,5-trihydroxy-6-(hydroxymethyl)tetrahydro-2H-pyran-2-yl)benzyl)phenyl)ethanone O-methyl oxime

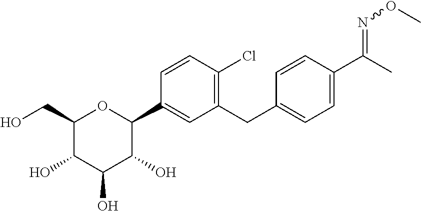 [SMILES: CON=C(C)c1ccc(Cc2cc([C@@H]3O[C@H](CO)[C@@H](O)[C@H](O)[C@H]3O)ccc2Cl)cc1]

### Step 1: Preparation of (2S,3R,4S,5S,6R)-2-(3-(4-((tert-butyldimethylsilyl)oxy)benzyl)-4-chlorophenyl)-6-(hydroxymethyl)-2-methoxytetrahydro-2H-pyran-3,4,5-triol

To a solution of (4-(5-bromo-2-chlorobenzyl)phenoxy)(tert-butyl)dimethylsilane (67.0 g, 0.163 mol, prepared by following the procedure given in US20070049537) in dry THF (1.0 L), ^{t}BuLi (228 mL, 0.342 mol, 1.6 M solution in pentane) was added at −78° C. The reaction mixture was stirred at the same temperature for 30 min. A solution of (3R,4S,5R,6R)-3,4,5-tris((trimethylsilyl)oxy)-6-(((trimethylsilyl)oxy)methyl)tetrahydro-2H-pyran-2-one (77.8 g, 0.167 mol, prepared following the procedure given in US20070049537) in dry THF (272 mL) was introduced into it at the same temperature and stirred for another 4 h. A solution of methanesulfonic acid (18.35 mL, 0.283 mol) in methanol (586 mL) was added and the temperature was raised to r.t. gradually and stirred for 16 h. The reaction was quenched by the addition of saturated aqueous solution of NaHCO₃ (500 mL) and volatiles were evaporated in vacuo. The residue obtained was dissolved in ethylacetate (1 L), washed with water (500 mL) and brine (500 mL) successively. The organic layer was dried over anhydrous Na₂SO₄ and volatiles were evaporated in vacuo. The residue obtained was purified by column chromatography (silica gel, 1:9 MeOH:DCM) to provide title compound (47.37 g, 55.4%) as off-white solid.

ESIMS (m/z): 524.2 (M−1)

### Step 2: Preparation of (2S,3R,4S,5R,6R)-6-(acetoxymethyl)-2-(3-(4-((tert-butyldimethylsilyl)oxy)benzyl)-4-chlorophenyl)-2-methoxytetrahydro-2H-pyran-3,4,5-triyl triacetate

To a solution of (2S,3R,4S,5S,6R)-2-(3-(4-((tert-butyldimethylsilyl)oxy)benzyl)-4-chlorophenyl)-6-(hydroxymethyl)-2-methoxytetrahydro-2H-pyran-3,4,5-triol (47.0 g, 0.090 mol) in dry THF (286 mL), diisopropylethylamine (114.28 mL, 0.660 mol) and 4-dimethylaminopyridine (3.94 g, 0.032 mol) were added. Acetic anhydride (55 mL, 0.582 mol) was added to the resulting solution at 0° C. The reaction mixture was stirred at r.t. for 2 h. Ethylacetate (1 L) was added to the reaction mixture, washed it with 2% HCl solution (2×100 mL), water (500 mL) and brine (500 mL) successively. The organic layer was dried over anhydrous Na₂SO₄ and volatiles were evaporated in vacuo. The residue obtained was purified by column chromatography (silica gel, 1:9 MeOH:DCM) to provide title compound (53.9 g, 86.93%) as off-white solid.

ESIMS (m/z): 715.0 (M+23)

### Step 3: Preparation of (2R,3R,4R,5S,6S)-2-(acetoxymethyl)-6-(4-chloro-3-(4-hydroxybenzyl)phenyl)tetrahydro-2H-pyran-3,4,5-triyl triacetate

To a solution of (2S,3R,4S,5R,6R)-6-(acetoxymethyl)-2-(3-(4-((tert-butyldimethylsilyl)oxy)benzyl)-4-chlorophenyl)-2-methoxytetrahydro-2H-pyran-3,4,5-triyl triacetate (44.0 g, 0.064 mol) in acetonitrile (200 mL), water (1.15 mL, 0.064 mol), triethylsilane (32.45 mL, 0.203 mol) and borontrifluoroetherate (15.94 mL, 0.127 mol) were added at 10° C. The resulting mixture was stirred at r.t. for 16 h. Additional amounts of triethylsilane (3.25 mL, 0.020 mol) and borontrifluoroetherate (1.59 mL, 0.013 mol) were added at 10° C. and heated at 30° C. for 6 h. Ethylacetate (1 L) was added to the reaction mixture, washed it with saturated NaHCO₃ solution (2×500 mL), water (500 mL) and brine (500 mL) successively. The organic layer was dried over anhydrous Na₂SO₄ and volatiles were evaporated in vacuo. The residue obtained was purified by column chromatography (silica gel, 4:6 Ethylacetate:Pet.Ether) to provide title compound (30 g, 86.27%) as off-white solid.

ESIMS (m/z): 548.2 (M−1)

### Step 4: Preparation of (2R,3R,4R,5S,6R)-2-(acetoxymethyl)-6-(4-chloro-3-(4-(trifluoro methylsulfonyloxy)benzyl)phenyl)tetrahydro-2H-pyran-3,4,5-triyl triacetate

To a solution of (2R,3R,4R,5S,6S)-2-(acetoxymethyl)-6-(4-chloro-3-(4-hydroxybenzyl)phenyl)tetrahydro-2H-pyran-3,4,5-triyl triacetate (1 g, 1.83 mmol) in dry DCM (15 mL), was added NEt₃ (0.76 mL, 5.46 mmol) followed by addition of triflic anhydride (0.51 mL, 3.09 mmol) at 0° C. The reaction was stirred at r.t. for 2 h. After the completion of reaction as observed by TLC, the reaction mixture was diluted with ethyl acetate (100 mL), washed with water (2×30 mL) and dried over Na₂SO₄. The combined organic layers were evaporated in vacuo to obtain the crude compound which was purified by column chromatography (silica gel, 1.5:8.5 Acetone:Pet. Ether) to provide the title compound (1 g, 80.6%).

ESIMS (m/z): 681.2 (M+1)

### Step 5: Preparation of (2R,3R,4R,5S,6S)-2-(acetoxymethyl)-6-(4-chloro-3-(4-cyanobenzyl)phenyl)tetrahydro-2H-pyran-3,4,5-triyl triacetate

To a solution of (2R,3R,4R,5S,6R)-2-(acetoxymethyl)-6-(4-chloro-3-(4-(trifluoromethyl sulfonyloxy)benzyl)phenyl)tetrahydro-2H-pyran-3,4,5-triyl triacetate (1 g, 1.46 mmol) in dry DMF (10 mL) was added tetrakis(triphenylphosphine)palladium(0) (848 mg, 0.73 mmol) followed by addition of zinc cyanide (517 mg, 4.4 mmol), 1,1′-bis(diphenylphosphino)ferrocene (407 mg, 0.73 mmol). The reaction mixture was refluxed at 150° C. for 3 h. After the completion of reaction as observed by TLC, the reaction mixture was cooled to r.t., diluted with ethyl acetate (100 mL), washed with water (2×20 mL) and dried over Na₂SO₄. The combined organic layers were evaporated in vacuo to obtain the crude compound which was purified by column chromatography (silica gel, 2:8 Acetone:Pet. Ether) to provide the title compound (600 mg, 73.2%).

ESIMS (m/z): 557.1 (M−1)

### Step 6: Preparation of 4-(2-chloro-5-((2S,3R,4R,5S,6R)-3,4,5-trihydroxy-6-(hydroxymethyl)tetrahydro-2H-pyran-2-yl)benzyl)benzonitrile

To a solution of (2R,3R,4R,5S,6S)-2-(acetoxymethyl)-6-(4-chloro-3-(4-cyanobenzyl)phenyl)tetrahydro-2H-pyran-3,4,5-triyl triacetate (600 mg, 1.08 mmol) in THF (3.5 mL), methanol (5.1 mL) and water (1.7 mL) was added lithium hydroxide monohydrate (90 mg, 2.15 mmol) at 0° C. The reaction was stirred at r.t. for 1 h. volatiles were evaporated in vacuo and the residue obtained was used as such in the next step.

ESIMS (m/z): 389 (M−1)

### Step 7: Preparation of 1-(4-(2-chloro-5-((2S,3R,4R,5S,6R)-3,4,5-trihydroxy-6-(hydroxymethyl)tetrahydro-2H-pyran-2-yl)benzyl)phenyl)ethanone

To a solution of 4-(2-chloro-5-((2S,3R,4R,5S,6R)-3,4,5-trihydroxy-6-(hydroxymethyl)tetrahydro-2H-pyran-2-yl)benzyl)benzonitrile (335 mg, 0.86 mmol) in THF (10 mL) and toluene (10 mL) was added 1.0 M methyl magnesium iodide in ether (4.3 mL, 4.3 mmol) at r.t. The reaction mixture was refluxed at 90° C. for 12 h. After the completion of reaction as observed by TLC, the reaction mixture was cooled to r.t., and aqueous ammonium chloride (10 mL) was added. The reaction mixture was diluted with ethyl acetate (50 mL), washed with water (2×20 mL) and dried over Na₂SO₄. The combined organic layers were evaporated in vacuo to obtain the crude compound which was purified by column chromatography (silica gel, 1:9 MeOH:DCM) to provide the title compound (209 mg, 60%).

ESIMS (m/z): 407.9 (M+1)

### Step 8: Preparation of 1-(4-(2-chloro-5-((2S,3R,4R,5S,6R)-3,4,5-trihydroxy-6-(hydroxy methyl)tetrahydro-2H-pyran-2-yl)benzyl)phenyl)ethanone O-methyl oxime

To a solution of 1-(4-(2-chloro-5-((2S,3R,4R,5S,6R)-3,4,5-trihydroxy-6-(hydroxymethyl)tetrahydro-2H-pyran-2-yl)benzyl)phenyl)ethanone (209 mg, 0.52 mmol), in ethanol (10 mL) under nitrogen atmosphere, were added pyridine (0.20 mL, 2.57 mmol), sodium acetate (421 mg, 5.14 mmol) and O-methylhydroxylamine hydrochloride (214 mg, 2.57 mmol). The reaction mixture was refluxed for 5 h. After completion of the reaction as confirmed by TLC, the solvent was evaporated. The crude compound was extracted with ethyl acetate (3×25 mL). The organic layer was separated, washed with water (3×10 mL), dried over Na₂SO₄ and concentrated in vacuo to afford crude product which was purified by column chromatography (silica gel, 1:9 MeOH:DCM) to afford the title compound (138 mg, 69%).

ESIMS (m/z): 436.7 (M+1)

¹H NMR (400 MHz, MeOD): δ 2.17 (s, 3H), 3.26-3.29 (m, 1H), 3.38-3.39 (m, 2H), 3.42-3.44 (m, 1H), 3.66-3.70 (m, 1H), 3.85-3.88 (m, 1H), 3.92 (s, 3H), 4.07-4.16 (m, 3H), 7.20 (d, J=8.3 Hz, 2H), 7.29 (dd, J=6.3 and 1.9 Hz, 1H), 7.34-7.36 (m, 2H), 7.55 (d, J=8.3 Hz, 1H)

### Example 3

### Preparation of 1-(4-(2-chloro-5-((1S,2S,3S,4R,5S)-2,3,4-trihydroxy-1-(hydroxymethyl)-6,8-dioxabicyclo[3.2.1]octan-5-yl)benzyl)phenyl)propan-1-one O-methyl oxime

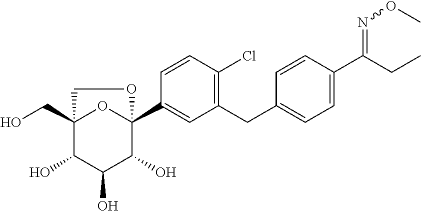 [SMILES: CCC(=NOC)c1ccc(Cc2cc([C@]34OC[C@](CO)(O3)[C@@H](O)[C@H](O)[C@H]4O)ccc2Cl)cc1]

### Step 1: Preparation of 2-(4-(allyloxy)benzyl)-4-bromo-1-chlorobenzene

To a solution of 4-(5-bromo-2-chlorobenzyl)phenol (6 g, 20.16 mmol, prepared as described in WO2006120208) in dry DMF (25 mL), was added Cs₂CO₃ (18.2 g, 60.6 mmol) and allyl bromide (4.2 mL, 50.6 mmol) at 0° C. and the reaction stirred at r.t. for 2 h. After the completion of the reaction as monitored by TLC, the reaction mixture was diluted with ethyl acetate (200 mL) and washed with water (3×30 mL). The combined organic layers were dried over Na₂SO₄ and the volatiles were evaporated in vacuo. The residue obtained was purified by column chromatography (silica gel, 0.4:9.6 Ethyl acetate:Pet. Ether) to afford the title compound (6.1 g, 89.6%).

### Step 2: Preparation of (2S,3R,4S,5S,6R)-2-(3-(4-(allyloxy)benzyl)-4-chlorophenyl)-6-(hydroxymethyl)-2-methoxytetrahydro-2H-pyran-3,4,5-triol

To a solution of 2-(4-(allyloxy)benzyl)-4-bromo-1-chlorobenzene (2.0 g, 5.92 mmol, prepared as described in Intermediate 1) in dry THF (50 mL), under nitrogen atmosphere, was added ⁿBuLi (4.8 mL, 7.7 mmol, 1.6 M solution in hexanes) at −78° C. The reaction mixture was stirred at the same temperature for 30 min A solution of (3R,4S,5R,6R)-3,4,5-tris((trimethylsilyl)oxy)-6-(((trimethylsilyl)oxy)methyl)tetrahydro-2H-pyran-2-one (3.6 g, 7.7 mol, prepared following the procedure given in US20070049537) in dry THF (40 mL) was added dropwise at the same temperature and stirred for another 3 h. After the completion of the reaction as confirmed by TLC, a solution of methanesulfonic acid (0.76 mL, 11.8 mmol) in methanol (30 mL) was added and the temperature was gradually raised to r.t. and stirred for 16 h. The reaction was quenched by the addition of triethylamine (3.3 mL) and volatiles were evaporated in vacuo. The residue obtained was dissolved in ethylacetate (300 mL) and washed with water (3×20 mL). The organic layers were dried over anhydrous Na₂SO₄ and volatiles were evaporated in vacuo. The residue obtained was purified by column chromatography (silica gel, 0.4:9.6 MeOH:DCM) to provide title compound (1.2 g, 45%).

ESIMS (m/z): 451.5 (M+1)

### Step 3: Preparation of ((2R,3S,4S,5R,6S)-6-(3-(4-(allyloxy)benzyl)-4-chlorophenyl)-3,4,5-trihydroxy-6-methoxytetrahydro-2H-pyran-2-yl)methyl 4-methylbenzenesulfonate

To a solution of (2S,3R,4S,5S,6R)-2-(3-(4-(allyloxy)benzyl)-4-chlorophenyl)-6-(hydroxymethyl)-2-methoxytetrahydro-2H-pyran-3,4,5-triol (1.2 g, 2.66 mmol) in leutidine (18.6 mL, 159.8 mmol), was added tosyl chloride (3.0 g, 15.98 mmol) at 0° C. The reaction mixture was stirred at r.t. for 12 h. After the completion of reaction as monitored by TLC, the reaction was diluted with ethylacetate (50 mL) and washed with 5% HCl (3×10 mL). The organic layers were dried over anhydrous Na₂SO₄ and volatiles were evaporated in vacuo. The residue obtained was purified by column chromatography (silica gel, 0.7:9.3 MeOH:DCM) to provide title compound (1.42 g, 89%).

### Step 4: Preparation of (2S,3S,4S,5R,6S)-6-(3-(4-(allyloxy)benzyl)-4-chlorophenyl)-3,4,5-trihydroxy-6-methoxytetrahydro-2H-pyran-2-carbaldehyde

To a solution of ((2R,3S,4S,5R,6S)-6-(3-(4-(allyloxy)benzyl)-4-chlorophenyl)-3,4,5-trihydroxy-6-methoxytetrahydro-2H-pyran-2-yl)methyl 4-methylbenzenesulfonate (4.5 g, 7.49 mmol) in dry DMSO (30 mL), was added collidine (6.95 mL, 52.44 mmol) and the mixture was heated at 150° C. for 45 min. After the completion of the reaction as monitored by TLC, the reaction mixture was used as such for the next step.

### Step 5: Preparation of (2S,3R,4S,5S)-2-(3-(4-(allyloxy)benzyl)-4-chlorophenyl)-6,6-bis(hydroxymethyl)-2-methoxytetrahydro-2H-pyran-3,4,5-triol

The above reaction mixture was diluted with EtOH (67 mL) and heated to 55° C. To it was added para-formaldehyde (5.62 g, 187.25 mmol), followed by dropwise addition of 21% solution of sodium ethoxide in EtOH (22 mL). The resulting mixture was heated at 55° C. for 20 h. After the completion of the reaction as monitored by TLC, the reaction was quenched with a solution of NaHSO₃ (15.5 g, 149.8 mmol in 110 mL of water) and stirred for 20 min. The solvent was evaporated under reduced pressure and then diluted with ethyl acetate (300 mL) and washed with water (2×30 mL). The combined organic layers were dried over anhydrous Na₂SO₄ and volatiles were evaporated in vacuo. The residue obtained was purified by column chromatography (silica gel, 0.7:9.3 MeOH:DCM) to provide title compound (0.6 g, 16.5% over two steps).

ESIMS (m/z): 480.5 (M+)

### Step 6: Preparation of (1S,2S,3S,4R,5S)-5-(3-(4-(allyloxy)benzyl)-4-chlorophenyl)-1-(hydroxymethyl)-6,8-dioxabicyclo[3.2.1]octane-2,3,4-triol

To a solution of (2S,3R,4S,5S)-2-(3-(4-(allyloxy)benzyl)-4-chlorophenyl)-6,6-bis(hydroxymethyl)-2-methoxytetrahydro-2H-pyran-3,4,5-triol (0.5 g, 1.04 mmol) in dry DCM (20 mL), was added trifluoracetic acid (4.6 mL, 62.43 mmol) dropwise at 0° C. and the reaction stirred at r.t. for 1 h. After the completion of the reaction as monitored by TLC, the reaction mixture was diluted with ethyl acetate (100 mL) and washed sequentially with water (2×10 mL) and NaHCO₃ (2×10 mL). The combined organic layers were dried over anhydrous Na₂SO₄ and volatiles were evaporated in vacuo. The crude compound was used as such in the next reaction (440 mg, 94.3%).

ESIMS (m/z): 448.0 (M+)

### Step 7: Preparation of (1R,2S,3S,4R,5S)-1-(acetoxymethyl)-5-(3-(4-(allyloxy)benzyl)-4-chlorophenyl)-6,8-dioxabicyclo[3.2.1]octane-2,3,4-triyl triacetate

To a solution of (1S,2S,3S,4R,5S)-5-(3-(4-(allyloxy)benzyl)-4-chlorophenyl)-1-(hydroxymethyl)-6,8-dioxabicyclo[3.2.1]octane-2,3,4-triol (650 mg, 1.44 mmol) in dry THF (20 mL), was added DIPEA (1.75 mL, 10.08 mmol) followed by DMAP (66 mg, 0.525 mmol). The reaction was cooled to 0° C. and acetic anhydride (0.68 mL, 7.24 mmol) was added dropwise. The reaction was stirred at r.t. for 1 h. After the completion of the reaction as observed by TLC, the reaction was diluted with ethyl acetate (100 mL) and washed with water (3×10 mL). The combined organic layers were dried over anhydrous Na₂SO₄ and volatiles were evaporated in vacuo. The residue obtained was purified by column chromatography (silica gel, 1.7:8.3 Acetone:Pet. Ether) to provide title compound (0.6 g, 67.5%).

ESIMS (m/z): 635.2 (M+18)

### Step 8: Preparation of (1R,2S,3S,4R,5S)-1-(acetoxymethyl)-5-(4-chloro-3-(4-hydroxybenzyl)phenyl)-6,8-dioxabicyclo[3.2.1]octane-2,3,4-triyl triacetate

To a solution of (1R,2S,3S,4R,5S)-1-(acetoxymethyl)-5-(3-(4-(allyloxy)benzyl)-4-chlorophenyl)-6,8-dioxabicyclo[3.2.1]octane-2,3,4-triyl triacetate (0.6 g, 0.973 mmol) in dry THF (10 mL), was added Pd(PPh₃)₄ (337 mg, 0.29 mmol) followed by 1,3-dimethyl barbituric acid (1.57 g, 9.73 mmol). The reaction mixture was then refluxed at 90° C. for 1 h. After the completion of reaction as monitored by TLC, the reaction was quenched with saturated solution of NaHCO₃ (10 mL), diluted with ethyl acetate (100 mL) and then washed with water (3×10 mL). The combined organic layers were dried over anhydrous Na₂SO₄ and volatiles were evaporated in vacuo. The residue obtained was purified by column chromatography (silica gel, 5:5 Ethyl acetate:Pet. Ether) to provide title compound (0.53 g, 94%).

ESIMS (m/z): 594.6 (M+18)

### Step 9: Preparation of (1R,2S,3S,4R,5S)-1-(acetoxymethyl)-5-(4-chloro-3-(4-(trifluoromethylsulfonyloxy)benzyl)phenyl)-6,8-dioxabicyclo[3.2.1]octane-2,3,4-triyl triacetate

To a solution of (1R,2S,3S,4R,5S)-1-(acetoxymethyl)-5-(4-chloro-3-(4-hydroxybenzyl)phenyl)-6,8-dioxabicyclo[3.2.1]octane-2,3,4-triyl triacetate (530 mg, 0.919 mmol) in dry DCM (10 mL), was added NEt₃ (0.51 mL, 3.66 mmol), followed by addition of triflic anhydride (0.30 mL, 1.83 mmol) at 0° C. The reaction was stirred at r.t. for 2 h. After the completion of reaction as observed by TLC, the reaction mixture was diluted with ethyl acetate (50 mL), washed with water (2×10 mL) and dried over Na₂SO₄. The combined organic layers were evaporated in vacuo to obtain the crude compound which was purified by column chromatography (silica gel, 1.5:8.5 Acetone:Pet. Ether) to provide the title compound (580 mg, 89%). ESIMS (m/z): 726.8 (M+18)

### Step 10: Preparation of (1R,2S,3S,4R,5S)-1-(acetoxymethyl)-5-(4-chloro-3-(4-cyanobenzyl)phenyl)-6,8-dioxabicyclo[3.2.1]octane-2,3,4-triyl triacetate

To a solution of (1R,2S,3S,4R,5S)-1-(acetoxymethyl)-5-(4-chloro-3-(4-trifluoromethylsulfonyloxy)benzyl)phenyl)-6,8-dioxabicyclo[3.2.1]octane-2,3,4-triyl triacetate (0.32 g, 0.45 mmol) in dry DMF (5 mL) was added tetrakis(triphenylphosphine) palladium(0) (156 mg, 0.135 mmol) followed by addition of zinc cyanide (159 mg, 1.35 mmol), 1,1′bis(diphenylphosphino)ferrocene (75 mg, 0.14 mmol). The reaction mixture was refluxed at 150° C. for 3 h. After the completion of reaction as observed by TLC, the reaction mixture was cooled to r.t., diluted with ethyl acetate (50 mL), washed with water (2×10 mL) and dried over Na₂SO₄. The combined organic layers were evaporated in vacuo to obtain the crude compound which was purified by column chromatography (silica gel, 1.5:8.5 Acetone:Pet. Ether) to provide the title compound (200 mg, 75.7%).

ESIMS (m/z): 587.1 (M+1)

### Step 11: Preparation of 4-(2-chloro-5-((1S,2S,3S,4R,5S)-2,3,4-trihydroxy-1-(hydroxymethyl)-6,8-dioxabicyclo[3.2.1]octan-5-yl)benzyl)benzonitrile

To a solution of (1R,2S,3S,4R,5S)-1-(acetoxymethyl)-5-(4-chloro-3-(4-cyanobenzyl)phenyl)-6,8-dioxabicyclo[3.2.1]octane-2,3,4-triyl triacetate (200 mg, 0.34 mmol) in THF (1.12 mL), methanol (1.68 mL) and water (0.56 mL) was added lithium hydroxide monohydrate (29 mg, 0.69 mmol) at 0° C. The reaction was stirred at r.t. for 1 h. volatiles were evaporated in vacuo and the residue obtained was purified by column chromatography (silica gel, 0.7:9.3 MeOH:DCM) to provide the title compound (130 mg, 91.3%).

### Step 12: Preparation of 1-(4-(2-chloro-5-((1S,2S,3S,4R,5S)-2,3,4-trihydroxy-1-(hydroxymethyl)-6,8-dioxabicyclo[3.2.1]octan-5-yl)benzyl)phenyl)propan-1-one

To a solution of 4-(2-chloro-5-((1S,2S,3S,4R,5S)-2,3,4-trihydroxy-1-(hydroxymethyl)-6,8-dioxabicyclo[3.2.1]octan-5-yl)benzyl)benzonitrile (130 mg, 0.31 mmol) in THF (2 mL) and toluene (5 mL) was added 1.0 Methyl magnesium iodide in ether (6 mL, 6.22 mmol) at r.t. The reaction mixture was refluxed at 80° C. for 15 h. After the completion of reaction as observed by TLC, the reaction mixture was cooled to r.t., and aqueous ammonium chloride (10 mL) was added. The reaction mixture was diluted with ethyl acetate (50 mL), washed with water (2×20 mL) and dried over Na₂SO₄. The combined organic layers were evaporated in vacuo to obtain the crude compound which was used as such in the next step.

ESIMS (m/z): 447.9 (M−1)

### Step 13: Preparation of (1R,2S,3S,4R,5S)-1-(acetoxymethyl)-5-(4-chloro-3-(4-propionylbenzyl)phenyl)-6,8-dioxabicyclo[3.2.1]octane-2,3,4-triyl triacetate

To a solution of 1-(4-(2-chloro-5-((1S,2S,3S,4R,5S)-2,3,4-trihydroxy-1-(hydroxymethyl)-6,8-dioxabicyclo[3.2.1]octan-5-yl)benzyl)phenyl)propan-1-one, obtained in the above step in dry THF (10 mL), were added, diisopropylethylamine (0.38 mL, 2.17 mmol) and 4-dimethylaminopyridine (15.8 mg, 0.124 mmol). Acetic anhydride (0.14 mL, 1.55 mmol) was added to the resulting solution at 0° C. The reaction mixture was stirred at r.t. for 30 min. After the completion of the reaction as observed by TLC, ethyl acetate (100 mL) was added to the reaction mixture, and it was washed with water (2×20 mL). The organic layer was dried over anhydrous Na₂SO₄ and volatiles were evaporated in vacuo. The residue obtained was purified by column chromatography (silica gel, 1.5:8.5 Acetone:Pet. Ether) to provide title compound (110 mg, 57.2% over steps 12 and 13).

ESIMS (m/z): 639.9 (M+23)

### Step 14: Preparation of (1R,2S,3S,4R,5S)-1-(acetoxymethyl)-5-(4-chloro-3-(4-1-(methoxyimino)propyl)benzyl)phenyl)-6,8-dioxabicyclo[3.2.1]octane-2,3,4-triyl triacetate

To a solution of (1R,2S,3S,4R,5S)-1-(acetoxymethyl)-5-(4-chloro-3-(4-propionylbenzyl)phenyl)-6,8-dioxabicyclo[3.2.1]octane-2,3,4-triyl triacetate (110 mg, 0.18 mmol), in ethanol (5 mL) under nitrogen atmosphere, were added pyridine (0.07 mL, 0.90 mmol), sodium acetate (146 mg, 1.78 mmol) and O-methylhydroxylamine hydrochloride (75 mg, 0.90 mmol). The reaction mixture was refluxed for 5 h. After completion of the reaction as confirmed by TLC, the solvent was evaporated. The crude compound was extracted with ethyl acetate (3×25 mL). The organic layer was separated, washed with water (3×10 mL), dried over Na₂SO₄ and concentrated in vacuo to afford crude product which was purified by column chromatography (silica gel, 1.5:8.5 Acetone:Pet. Ether) to afford the title compound (70 mg, 61%). ESIMS (m/z): 668.9 (M+23)

### Step 15: Preparation of 1-(4-(2-chloro-5-((1S,2S,3S,4R,5S)-2,3,4-trihydroxy-1-(hydroxymethyl)-6,8-dioxabicyclo[3.2.1]octan-5-yl)benzyl)phenyl)propan-1-one O-methyl oxime

To a solution of (1R,2S,3S,4R,5S)-1-(acetoxymethyl)-5-(4-chloro-3-(4-1-(methoxyimino) propyl)benzyl)phenyl)-6,8-dioxabicyclo[3.2.1]octane-2,3,4-triyl triacetate (70 mg, 0.11 mmol) in THF (0.36 mL), methanol (0.54 mL) and water (0.18 mL) was added lithium hydroxide monohydrate (9.1 mg, 0.22 mmol) at 0° C. The reaction was stirred at r.t. for 1 h. After completion of the reaction as observed by TLC, volatiles were evaporated in vacuo and the residue obtained was purified by column chromatography (silica gel, 0.6:9.4 MeOH:DCM) to provide the title compound (47 mg, 91%).

ESIMS (m/z): 478.6 (M+1)

¹H NMR (400 MHz, CD₃OD): δ 1.70 (t, J=7.6 Hz, 3H), 2.72 (q, J=7.6 Hz, 2H), 3.54 (d, J=7.9 Hz, 1H), 3.59 (dd, J=7.5 and 1.2 Hz, 1H), 3.62-3.69 (m, 2H), 3.77 (dd, J=8.0 and 1.0 Hz, 1H), 3.83 (d, J=12.5 Hz, 1H), 3.91 (s, 3H), 4.12-4.15 (m, 3H), 7.19-7.21 (m, 2H), 7.35-7.41 (m, 2H), 7.49-7.50 (d, J=1.1 Hz, 1H), 7.52-7.54 (m, 2H)

### Example 4

### Preparation of 1-(4-(2-chloro-5-((1S,2S,3S,4R,5S)-2,3,4-trihydroxy-1-(hydroxymethyl)-6,8-dioxabicyclo[3.2.1]octan-5-yl)benzyl)phenyl)ethanone O-methyl oxime

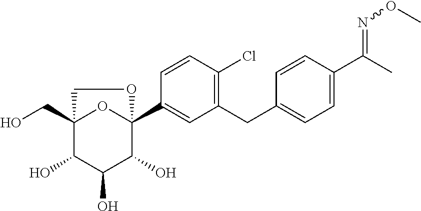 [SMILES: CON=C(C)c1ccc(Cc2cc([C@]34OC[C@](CO)(O3)[C@@H](O)[C@H](O)[C@H]4O)ccc2Cl)cc1]

### Step 1: Preparation of (1R,2S,3S,4R,5S)-1-(acetoxymethyl)-5-(4-chloro-3-(4-((trimethylsilyl)ethynyl)benzyl)phenyl)-6,8-dioxabicyclo[3.2.1]octane-2,3,4-triyl triacetate

To a solution of (1R,2S,3S,4R,5S)-1-(acetoxymethyl)-5-(4-chloro-3-(4-(trifluoromethylsulfonyloxy)benzyl)phenyl)-6,8-dioxabicyclo[3.2.1]octane-2,3,4-triyl triacetate (580 mg, 0.818 mmol, obtained in Step 9 of EXAMPLE 3) in dry DMF (2.7 mL), was added Pd(PPh₃)₂Cl₂ (57.5 mg, 0.081 mmol), CuI (31.15 mg, 0.163 mmol), triethylamine (0.40 mL, 2.90 mmol) followed by TMS acetylene (0.342 mL, 2.47 mmol). The solution was degassed under Argon and the reaction mixture was heated at 90° C. for 5 h. To the reaction mixture was again added Pd(PPh₃)₂Cl₂ (57.5 mg, 0.08 mmol) and TMS acetylene (0.34 mL, 2.47 mmol). The heating was continued at 90° C. for 12 h. After the completion of the reaction as confirmed by TLC, the reaction mixture was diluted with ethyl acetate (50 mL) and given water washings (2×10 mL). The combined organic layers were dried over Na₂SO₄ and concentrated in vacuo to afford crude product which was purified by column chromatography (silica gel, 1.3:8.7 Acetone:Pet. Ether) to afford the title compound (290 mg, 54%).

ESIMS (m/z): 674.9 (M+18)

### Step 2: Preparation of (1R,2S,3S,4R,5S)-1-(acetoxymethyl)-5-(3-(4-acetylbenzyl)-4-chlorophenyl)-6,8-dioxabicyclo[3.2.1]octane-2,3,4-triyl triacetate

(1R,2S,3S,4R,5S)-1-(acetoxymethyl)-5-(4-chloro-3-(4-((trimethylsilyl)ethynyl)benzyl)phenyl)-6,8-dioxabicyclo[3.2.1]octane-2,3,4-triyl triacetate (290 mg, 0.44 mmol) was taken in formic acid (1.41 mL) and refluxed at 100° C. for 30 min. After the completion of reaction as confirmed by TLC, the reaction mixture was diluted with ethyl acetate (50 mL) and given water washings (2×20 mL). The combined organic layers were dried over Na₂SO₄ and concentrated in vacuo to afford crude product which was purified by column chromatography (silica gel, 3:7 Pet. Ether:Ethyl acetate) to afford the title compound (210 mg, 78.9%).

ESIMS (m/z): 620.9 (M+18)

### Step 3: Preparation of (1R,2S,3S,4R,5S)-1-(acetoxymethyl)-5-(4-chloro-3-(4-(1-(methoxyimino)ethyl)benzyl)phenyl)-6,8-dioxabicyclo[3.2.1]octane-2,3,4-triyl triacetate

To a solution of (1R,2S,3S,4R,5S)-1-(acetoxymethyl)-5-(3-(4-acetylbenzyl)-4-chlorophenyl)-6,8-dioxabicyclo[3.2.1]octane-2,3,4-triyl triacetate (210 mg, 0.35 mmol), in ethanol (10 mL) under nitrogen atmosphere, were added pyridine (0.14 mL, 1.74 mmol), sodium acetate (285 mg, 3.48 mmol) and O-methylhydroxylamine hydrochloride (145.5 mg, 1.74 mmol). The reaction mixture was refluxed at 80° C. for 3 h. After completion of the reaction as confirmed by TLC, the solvent was evaporated. The crude compound was extracted with ethyl acetate (3×25 mL). The organic layer was separated, washed with water (3×20 mL), dried over Na₂SO₄ and concentrated in vacuo to afford crude product which was purified by column chromatography (silica gel, 1.5:8.5 Acetone:Pet ether) to afford the title compound (120 mg, 57.2%).

ESIMS (m/z): 632.9 (M+1)

### Step 4: Preparation of 1-(4-(2-chloro-5-((1S,2S,3S,4R,5S)-2,3,4-trihydroxy-1-(hydroxymethyl)-6,8-dioxabicyclo[3.2.1]octan-5-yl)benzyl)phenyl)ethanone O-methyl oxime

To a solution of (1R,2S,3S,4R,5S)-1-(acetoxymethyl)-5-(4-chloro-3-(4-(1-(methoxyimino)ethyl)benzyl)phenyl)-6,8-dioxabicyclo[3.2.1]octane-2,3,4-triyl triacetate (120 mg, 0.19 mmol) in THF (0.6 mL), methanol (0.9 mL) and water (0.3 mL) was added lithium hydroxide monohydrate (16 mg, 0.38 mmol) at 0° C. The reaction was stirred at r.t. for 1 h. After the completion of reaction as confirmed by TLC, volatiles were evaporated in vacuo and the residue obtained was purified by column chromatography (silica gel, 0.6:9.4 MeOH:DCM) to afford the title compound (70 mg, 79.47%).

ESIMS (m/z): 464.7 (M+1)

¹H NMR (400 MHz, CD₃OD): δ 2.17 (s, 3H), 3.52 (d, J=7.6 Hz, 1H), 3.58 (dd, J=7.6 and 1.2 Hz, 1H), 3.64 (t, J=8.1 Hz, 1H), 3.67 (d, J=12.5 Hz, 1H), 3.76 (d, J=8.3 Hz, 1H), 3.82 (d, J=12.5 Hz, 1H), 3.92 (s, 3H), 4.12 (s, 2H), 4.13 (d, J=7.6 Hz, 1H), 7.19 (d, J=8 Hz, 1H), 7.36 (d, J=8 Hz, 2H), 7.40 (dd, J=8.4 and 2 Hz, 1H), 7.48 (d, J=2 Hz, 1H), 7.53-7.56 (d, J=8.4 Hz, 2H)

### Example 5

### Preparation of 1-(4-(2-chloro-5-((2S,3R,4R,5S,6S)-3,4,5-trihydroxy-6-methoxy tetrahydro-2H-pyran-2-yl)benzyl)phenyl)ethanone O-ethyl oxime

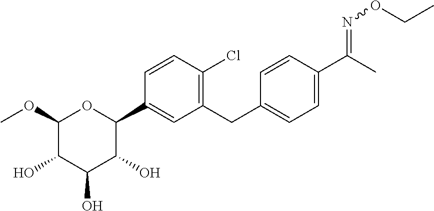 [SMILES: CCON=C(C)c1ccc(Cc2cc([C@@H]3O[C@H](OC)[C@@H](O)[C@H](O)[C@H]3O)ccc2Cl)cc1]

### Step 1: Preparation of (2S,3S,4R,5S,6S)-2-(4-chloro-3-(4-((trimethylsilyl)ethynyl)benzyl)phenyl)-6-methoxytetrahydro-2H-pyran-3,4,5-triyl triacetate

To a solution of (2S,3S,4R,5S,6S)-2-(4-chloro-3-(4-(((trifluoromethyl)sulfonyl)oxy)benzyl)phenyl)-6-methoxytetrahydro-2H-pyran-3,4,5-triyl triacetate (2.3 g, 3.599 mmol, step 5 of EXAMPLE 1) in dry DMF (12 mL), was added Pd(PPh₃)₄ (831.9 mg, 0.719 mmol), CuI (274.04 mg, 1.439 mmol), DIPEA (2.51 mL, 14.40 mmol) followed by TMS acetylene (1.99 mL, 14.396 mmol). The solution was purged with N₂ and heated at 100° C. for 4 h in a sealed vessel. To the reaction mixture was again added Pd(PPh₃)₄ (831.9 mg, 0.719 mmol) and TMS acetylene (1.99 mL, 14.396 mmol) and heating was continued at 100° C. for another 16 h. After the completion of the reaction as confirmed by TLC, the reaction mixture was diluted with ethyl acetate (50 mL) and given water washings (2×10 mL). The organic layer was dried over Na₂SO₄ and concentrated in vacuo to afford crude product which was purified by column chromatography (silica gel, 3:7 Ethyl acetate:Pet. Ether) to afford the title compound (1.95 g, 92.4%).

ESIMS (m/z): 606 (M+18)

### Step 2: Preparation of (2S,3S,4R,5S,6S)-2-(3-(4-acetylbenzyl)-4-chlorophenyl)-6-methoxytetrahydro-2H-pyran-3,4,5-triyl triacetate

(2S,3S,4R,5S,6S)-2-(4-chloro-3-(4-((trimethylsilyl)ethynyl)benzyl)phenyl)-6-methoxytetrahydro-2H-pyran-3,4,5-triyl triacetate (1.95 g, 3.321 mmol) was taken in formic acid (66.42 mL) and refluxed at 100° C. for 2 h. After the completion of reaction as confirmed by TLC, the reaction mixture was diluted with ethyl acetate (150 mL) and given water washings (2×50 mL). The organic layer was dried over Na₂SO₄ and concentrated in vacuo to afford crude product which was purified by column chromatography (silica gel, 3:7 Ethyl acetate:Pet. Ether) to afford the title compound (1.3 g, 73.44%).

ESIMS (m/z): 550 (M+18)

### Step 3: Preparation of 1-(4-(2-chloro-5-((2S,3R,4R,5S,6S)-3,4,5-trihydroxy-6-methoxytetrahydro-2H-pyran-2-yl)benzyl)phenyl)ethanone

To a solution of (2S,3S,4R,5S,6S)-2-(3-(4-acetylbenzyl)-4-chlorophenyl)-6-methoxytetrahydro-2H-pyran-3,4,5-triyl triacetate (400 mg, 0.75 mmol) in THF (3.75 mL), methanol (2.50 mL) and water (1.25 mL) was added lithium hydroxide monohydrate (47.23 mg, 1.125 mmol) at 0° C. The reaction was stirred at r.t. for 1 h. After the completion of reaction as confirmed by TLC, volatiles were evaporated in vacuo and the residue obtained was purified by column chromatography (silica gel, 1:9 MeOH:DCM) to afford the title compound (300 mg, 98.36%).

ESIMS (m/z): 424.5 (M+18), 429.5 (M+23)

### Step 4: Preparation of 1-(4-(2-chloro-5-((2S,3R,4R,5S,6S)-3,4,5-trihydroxy-6-methoxy tetrahydro-2H-pyran-2-yl)benzyl)phenyl)ethanone O-ethyl oxime

To a solution of 1-(4-(2-chloro-5-((2S,3R,4R,5S,6S)-3,4,5-trihydroxy-6-methoxytetrahydro-2H-pyran-2-yl)benzyl)phenyl)ethanone (130 mg, 0.32 mmol) in ethanol (1.6 mL) under nitrogen atmosphere, were added and K₂CO₃ (220.7 mg, 1.597 mmol) and O-ethylhydroxylamine hydrochloride (155.83 mg, 1.597 mmol) at 0° C. The resulting mixture was stirred at r.t. for 1 h. After completion of the reaction as confirmed by TLC, the reaction mixture was passed through celite plug and the filterate was evaporated. The crude product was purified by column chromatography (silica gel, 1:9 Methanol:DCM) to afford the title compound (80 mg, 55.65%).

ESIMS (m/z): 450.7 (M+1)

¹H NMR (400 MHz, CD₃OD): δ 1.29 (t, J=7.1 Hz, 3H), 2.18 (s, 3H), 3.26-3.34 (m, 2H), 3.38-3.46 (m, 1H), 3.47 (s, 3H), 4.07-4.20 (m, 5H), 4.30 (d, J=7.7 Hz, 1H), 7.21 (d, J=8.3 Hz, 2H), 7.26 (dd, J=8.2 and 2.0 Hz, 1H), 7.30 (d, J=1.9 Hz, 1H), 7.37 (d, J=8.2 Hz, 1H), 7.56 (d, J=8.4 Hz, 2H)

### Example 6

### Preparation of 1-(4-(2-chloro-5-((2S,3R,4R,5S,6R)-3,4,5-trihydroxy-6-(hydroxymethyl)tetrahydro-2H-pyran-2-yl)benzyl)phenyl)ethanone O-ethyl oxime

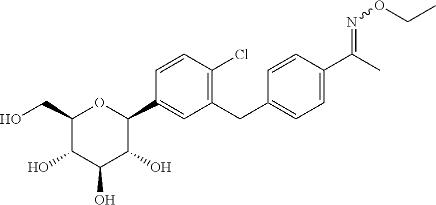 [SMILES: CCON=C(C)c1ccc(Cc2cc([C@@H]3O[C@H](CO)[C@@H](O)[C@H](O)[C@H]3O)ccc2Cl)cc1]

### Step 1: Preparation of (2R,3R,4R,5S,6S)-2-(acetoxymethyl)-6-(4-chloro-3-(4-((trimethylsilyl)ethynyl)benzyl)phenyl)tetrahydro-2H-pyran-3,4,5-triyl triacetate

To a solution of (2R,3R,4R,5S,6S)-2-(acetoxymethyl)-6-(4-chloro-3-(4-(((trifluoromethyl)sulfonyl)oxy)benzyl)phenyl)tetrahydro-2H-pyran-3,4,5-triyl triacetate (4.5 g, 6.77 mmol) obtained in step 4 of EXAMPLE 2, in dry DMF (24 mL), was added Pd(PPh₃)₂Cl₂ (475.17 mg, 0.677 mmol), CuI (257.87 mg, 1.35 mmol), triethylamine (3.34 mL, 24.03 mmol) followed by TMS acetylene (2.87 mL, 20.46 mmol). The solution was degassed under Argon and the reaction mixture was heated at 90° C. for 5 h. To the reaction mixture was again added Pd(PPh₃)₂Cl₂ (475.17 mg, 0.677 mmol) and TMS acetylene (2.87 mL, 20.46 mmol). The heating was continued at 90° C. for 12 h. After the completion of the reaction as confirmed by TLC, the reaction mixture was diluted with ethyl acetate (200 mL) and water (100 mL). The organic layer was separated, dried over Na₂SO₄ and concentrated in vacuo to afford crude product which was purified by column chromatography (silica gel, 1:9 Ethyl acetate:Pet. Ether) to afford the title compound (3.25 g, 76%) as a white solid.

ESIMS (m/z): 657(M⁺)

### Step 2: Preparation of (2R,3R,4R,5S,6S)-2-(acetoxymethyl)-6-(3-(4-acetylbenzyl)-4-chlorophenyl)tetrahydro-2H-pyran-3,4,5-triyl triacetate

(2R,3R,4R,5S,6S)-2-(acetoxymethyl)-6-(4-chloro-3-(4-((trimethylsilyl)ethynyl)benzyl) phenyl)tetrahydro-2H-pyran-3,4,5-triyl triacetate (3.0 g, 4.77 mmol) was taken in formic acid (95 mL) and refluxed at 100° C. for 30 min. After the completion of the reaction as confirmed by TLC, the reaction mixture was quenched by slow addition of sodium bicarbonate solution (250 mL). The crude compound was diluted with ethyl acetate (250 mL). The organic layer was separated, washed with water (100 mL), dried over Na₂SO₄ and concentrated in vacuo to afford crude product which was purified by column chromatography (silica gel, 2:8 Ethyl acetate:Pet. Ether) to afford the title compound (2.5 g, 92.5%) as a solid.

### Step 3: Preparation of (2R,3R,4R,5S,6S)-2-(acetoxymethyl)-6-(4-chloro-3-(4-(1-(ethoxyimino)ethyl)benzyl)phenyl)tetrahydro-2H-pyran-3,4,5-triyl triacetate

To a solution of (2R,3R,4R,5S,6S)-2-(acetoxymethyl)-6-(3-(4-acetylbenzyl)-4-chlorophenyl)tetrahydro-2H-pyran-3,4,5-triyl triacetate (400 mg, 0.69 mmol) in ethanol (5 mL) under nitrogen atmosphere, was added pyridine (0.24 mL, 2.78 mmol) and O-ethylhydroxylamine hydrochloride (135.0 mg, 1.39 mmol). The reaction mixture was refluxed at 80° C. for 4 h. After completion of the reaction as confirmed by TLC, the solvent was evaporated. The crude compound was extracted with ethyl acetate (2×20 mL). The organic layer was separated, washed with 5% HCl (10 mL), water (10 mL), dried over Na₂SO₄ and concentrated in vacuo to afford crude product which was purified by column chromatography (silica gel, 2:8 Ethyl acetate:Pet. Ether) to afford the title compound (300 mg, 69.7%) as a solid.

ESIMS (m/z): 618.7 (M+1)

### Step 4: Preparation of 1-(4-(2-chloro-5-((2S,3R,4R,5S,6R)-3,4,5-trihydroxy-6-(hydroxymethyl)tetrahydro-2H-pyran-2-yl)benzyl)phenyl)ethanone O-ethyl oxime

To a solution of (2R,3R,4R,5S,6S)-2-(acetoxymethyl)-6-(4-chloro-3-(4-(1-(ethoxyimino)ethyl)benzyl)phenyl)tetrahydro-2H-pyran-3,4,5-triyl triacetate (300 mg, 0.48 mmol) in THF (1.6 mL), methanol (2.4 mL) and water (0.8 mL) was added lithium hydroxide monohydrate (24.4 mg, 0.58 mmol) at 0° C. The reaction was stirred at r.t. for 1 h. After the completion of reaction as confirmed by TLC, volatiles were evaporated in vacuo and the compound was extracted with ethyl acetate (50 mL). The organic layer was separated, washed with water (10 mL), dried over Na₂SO₄ and concentrated to afford the title compound (280 mg, 82.5%) as a white solid.

ESIMS (m/z): 450.6 (M+1), 451.7 (M+2)

¹H NMR (400 MHz, CD₃OD): δ 1.25 (t, J=4.0 Hz, 3H), 2.17 (s, 3H), 3.26-3.28 (m, 1H), 3.35-3.39 (m, 2H), 3.42-3.47 (m, 1H), 3.67 (dd, J=12.0 and 5.2 Hz, 1H), 3.87 (dd, J=12.4 and 2.0

Hz, 1H), 4.08-4.15 (m, 3H), 4.17 (q, J=6.8 Hz, 2H), 7.19 (d, J=8.0 Hz, 2H), 7.28 (dd, J=8.0 and 4.0 Hz, 1H), 7.35 (dd, J=3.6 and 1.6 Hz, 2H), 7.54 (d, J=8.0 Hz, 1H)

### Example 7

### Preparation of 1-(4-(2-chloro-5-((2S,3R,4R,5S,6R)-3,4,5-trihydroxy-6-(hydroxymethyl)tetrahydro-2H-pyran-2-yl)benzyl)phenyl)-propan-1-one O-methyl oxime

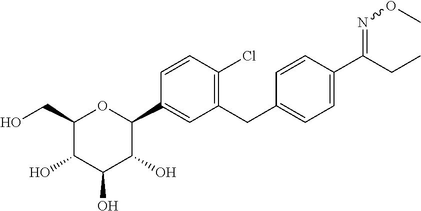 [SMILES: CCC(=NOC)c1ccc(Cc2cc([C@@H]3O[C@H](CO)[C@@H](O)[C@H](O)[C@H]3O)ccc2Cl)cc1]

### Step 1: Preparation of (2R,3R,4R,5S,6S)-2-(acetoxymethyl)-6-(4-chloro-3-(4-propionylbenzyl) phenyl)tetrahydro-2H-pyran-3,4,5-triyl triacetate

To a solution of (2R,3R,4R,5S,6S)-2-(acetoxymethyl)-6-(3-(bromomethyl)-4-chlorophenyl)tetrahydro-2H-pyran-3,4,5-triyl triacetate (Intermediate 1, 5 g, 9.36 mmol) and 1-(4-(4,4,5,5-tetramethyl-1,3,2-dioxaborolan-2-yl)phenyl)propan-1-one (Intermediate 2, 3.4 g, 13.1 mmol) in DMF (15 mL) and water (15 mL) were added Na₂CO₃ (2.97 g, 28.08 mmol) and Pd(dppf)₂Cl₂ (380 mg, 0.46 mmol). The resulting solution was purged with N₂ and heated at 100° C. for 1 h. After completion of the reaction as confirmed by TLC, the reaction mixture was diluted with ethyl acetate (200 mL) and washed with water (2×50 mL). The organic layer was dried over Na₂SO₄ and concentrated in vacuo to afford crude product which was purified by column chromatography (silica gel, 4:6 Ethyl acetate:Pet. Ether) to afford the title compound (4.5 g, 81%).

ESIMS (m/z): 589.8 (M+1)

### Step 2: Preparation of (2R,3R,4R,5S,6S)-2-(acetoxymethyl)-6-(4-chloro-3-(4-(1-(methoxyimino)-propyl)benzyl)phenyl)tetrahydro-2H-pyran-3,4,5-triyl triacetate

To a solution of (2R,3R,4R,5S,6S)-2-(acetoxymethyl)-6-(4-chloro-3-(4-propionylbenzyl) phenyl)tetrahydro-2H-pyran-3,4,5-triyl triacetate (1.3 g, 2.21 mmol), in ethanol (11 mL) under nitrogen atmosphere, were added and pyridine (0.89 mL, 11.05 mmol) and O-methylhydroxylamine hydrochloride (369 mg, 4.42 mmol). The reaction mixture was refluxed at 80° C. for 3 h. After completion of the reaction as confirmed by TLC, the solvent was evaporated. The crude compound was diluted with ethyl acetate (100 mL). The organic layer was washed with water (3×20 mL), dried over Na₂SO₄ and concentrated in vacuo to afford crude product which was purified by column chromatography (silica gel, 4:6 Ethyl acetate:Pet. Ether) to afford the title compound (1 g, 73.3%).

ESIMS (m/z): 618.4 (M+1)

### Step 3: Preparation of 1-(4-(2-chloro-5-((2S,3R,4R,5S,6R)-3,4,5-trihydroxy-6-(hydroxymethyl)tetrahydro-2H-pyran-2-yl)benzyl)phenyl)-propan-1-one O-methyl oxime

To a solution of (2R,3R,4R,5S,6S)-2-(acetoxymethyl)-6-(4-chloro-3-(4-(1-(methoxyimino)-propyl)benzyl)phenyl)tetrahydro-2H-pyran-3,4,5-triyl triacetate (500 mg, 0.81 mmol) in THF (1.82 mL), methanol (2.43 mL) and water (0.61 mL) was added lithium hydroxide monohydrate (51 mg, 1.21 mmol) at 0° C. The reaction was stirred at r.t. for 1 h. After the completion of reaction as confirmed by TLC, volatiles were evaporated in vacuo and the residue obtained was purified by column chromatography (silica gel, 1:9 MeOH:DCM) to afford the title compound (309.4 mg, 85%).

ESIMS (m/z): 450.1 (M+1)

¹H NMR (400 MHz, CD₃OD): δ 1.00 (t, J=7.4 Hz, 3H), 2.51 (q, J=7.5 Hz, 2H), 3.25-3.47 (m, 4H), 3.68 (dd, J=12.0 and 5.2 Hz, 1H), 3.70 (s, 3H), 3.85-3.88 (m, 1H), 4.07-4.17 (m, 3H), 7.22 (d, J=8.4 Hz, 2H), 7.27-7.30 (m, 3H), 7.36 (d, J=8.2 Hz, 1H), 7.38 (d, J=2.1 Hz, 1H)

### Example 8

### Preparation of 1-(4-(2-chloro-5-((1S,2S,3S,4R,5S)-2,3,4-trihydroxy-1-(hydroxymethyl)-6,8-dioxabicyclo[3.2.1]octan-5-yl)benzyl)phenyl)ethanone O-ethyl oxime

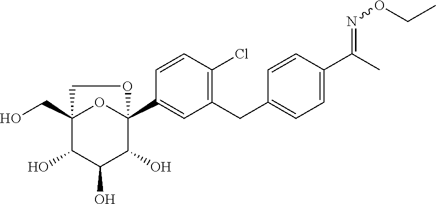 [SMILES: CCON=C(C)c1ccc(Cc2cc([C@]34OC[C@](CO)(O3)[C@@H](O)[C@H](O)[C@H]4O)ccc2Cl)cc1]

### Step 1: Preparation of ((2R,3R,4S,5R,6S)-6-(4-chloro-3-(phenoxymethyl)phenyl)-6-methoxy-3,4,5-tris((trimethylsilyl)oxy)tetrahydro-2H-pyran-2-yl)methanol

To a solution of (2S,3R,4S,5S,6R)-2-(4-chloro-3-(phenoxymethyl)phenyl)-6-(hydroxymethyl)-2-methoxytetrahydro-2H-pyran-3,4,5-triol (25 g, 60.90 mmol), (obtained in step 4 of Intermediate 1) in DCM (180 mL) under nitrogen atmosphere, was added imidazole (41.46 g, 609.01 mmol) and chlorotrimethyl silane (46.37 mL, 365.4 mmol) at 0° C. The reaction mixture was warmed to room temperature and stirred for 2 h. After the completion of the reaction as confirmed by TLC, water (50 mL) was added. The organic layer was separated, dried over Na₂SO₄ and concentrated to afford crude (((2S,3R,4S,5R,6R)-2-(4-chloro-3-(phenoxymethyl)phenyl)-2-methoxy-6-(((trimethylsilyl)oxy)methyl)tetrahydro-2H-pyran-3,4,5-triyl)tris(oxy))tris(trimethylsilane) (42.35 g, 100%) as a solid which was used for next step without any purification.

To the above suspension in MeOH (150 mL) under nitrogen atmosphere, was added potassium carbonate (168.08 mg, 1.218 mmol) at 0° C. The reaction mixture was warmed to room temperature and stirred for 2 h. After completion of the reaction as confirmed by TLC, the reaction mixture was cooled to 0° C. and quenched with a solution of acetic acid (0.07 ml, 1.217 mmol) in methanol (2 mL) followed by addition of water (200 mL). The crude compound was extracted with diethyl ether (500 mL). The organic layer was separated, washed with water (200 mL), dried over Na₂SO₄, concentrated in vacuo to afford crude product which was purified by column chromatography (silica gel, 4:6 Ethyl acetate:Pet. Ether) to afford the title compound (29 g, 76%) as a white solid.

ESIMS (m/z): 626.3 (M+)

### Step 2: Preparation of (2S,3R,4S,5R,6S)-6-(4-chloro-3-(phenoxymethyl)phenyl)-6-methoxy-3,4,5-tris((trimethylsilyl)oxy)tetrahydro-2H-pyran-2-carbaldehyde

To a suspension of ((2R,3R,4S,5R,6S)-6-(4-chloro-3-(phenoxymethyl)phenyl)-6-methoxy-3,4,5-tris((trimethylsilyl)oxy)tetrahydro-2H-pyran-2-yl)methanol (23.0 g, 36.7 mmol) in DMSO (250 mL) was added DCM (20 mL) to make a clear solution. The solution was cooled to 0° C. followed by addition of triethyl amine (122.4 ml, 881.0 mmol) and sulphur trioxide pyridine complex (87.5 g, 550.6 mmol). The reaction mixture was stirred at 0 to 10° C. for 3 h under nitrogen atmosphere. After completion of the reaction as confirmed by TLC, the crude compound was extracted with diethyl ether (1 L). The organic layer was separated, washed with water (2×500 mL), dried over Na₂SO₄, concentrated in vacuo to afford crude title compound (22.9 g, 100%) as a viscous oil which was used for next step without any purification.

### Step 3: Preparation of (2S,3R,4S,5S)-2-(4-chloro-3-(phenoxymethyl)phenyl)-6,6-bis(hydroxymethyl)-2-methoxytetrahydro-2H-pyran-3,4,5-triol

To a solution of (2S,3R,4S,5R,6S)-6-(4-chloro-3-(phenoxymethyl)phenyl)-6-methoxy-3,4,5-tris((trimethylsilyl)oxy)tetrahydro-2H-pyran-2-carbaldehyde (22.9 g, 36.7 mmol) in EtOH (92 mL) under nitrogen atmosphere, was added para-formaldehyde (33.0 g, 1.1 mol) at 55° C. followed by addition of 21% NaOEt in ethanol (23.12 g NaOEt/110 mL EtOH). The reaction mixture was heated at 55° C. overnight. After the completion of the reaction as confirmed by TLC, ethanol was evaporated. The crude compound was extracted with ethyl acetae (700 mL) followed by washing with sodium bisulphate solution (68.67 g, 660.6 mmol) in water (458.7 mL) The organic layer was separated, dried over Na₂SO₄ and concentrated in vacuo to afford crude compound, which was purified by column chromatography (silica gel, 6:4 MeOH:DCM) to obtain the compound as a white solid (12 g).

### Step 4: Preparation of (3S,4S,5R,6S)-2,2-bis(acetoxymethyl)-6-(4-chloro-3-(phenoxymethyl)phenyl)-6-methoxytetrahydro-2H-pyran-3,4,5-triyl triacetate

To a solution of the compound obtained above (12 g, 27.24 mmol) in THF (55 mL) under nitrogen atmosphere, was added DMAP (1.19 g, 9.80 mmol), DIPEA (34.24 mL, 199.68 mmol) and acetic anhydride (20.6 mL, 217.93 mmol) at 0° C. The reaction mixture was warmed to r.t. and stirred for 2 h. After the completion of reaction as confirmed by TLC, the crude compound was extracted with ethyl acetate (500 mL). The organic layer was washed with 10% HCl (200 mL), separated, dried over Na₂SO₄ and concentrated in vacuo to afford crude compound which was purified by column chromatography (silica gel, 2.5:7.5 Ethyl acetate:Pet. Ether) to afford (3S,4S,5R,6S)-2,2-bis(acetoxymethyl)-6-(4-chloro-3-(phenoxymethyl)phenyl)-6-methoxytetrahydro-2H-pyran-3,4,5-triyl triacetate (12.0 g, 50%) as a white solid.

ESIMS (m/z): 673.9 (M+23), 651.7 (M+1)

### Step 5: Preparation of (2S,3R,4S,5S)-2-(4-chloro-3-(phenoxymethyl)phenyl)-6,6-bis(hydroxymethyl)-2-methoxytetrahydro-2H-pyran-3,4,5-triol

To a solution of (3S,4S,5R,6S)-2,2-bis(acetoxymethyl)-6-(4-chloro-3-(phenoxymethyl)phenyl)-6-methoxytetrahydro-2H-pyran-3,4,5-triyl triacetate (12.0 g, 18.44 mmol) obtained above, in THF (60.8 mL), methanol (90 mL) and water (30.6 mL), was added lithium hydroxide monohydrate (929 mg, 22.12 mmol) at 0° C. The reaction mixture was stirred at r.t. for 1 h. After the completion of the reaction as confirmed by TLC, volatiles were evaporated in vacuo and the compound was extracted with ethyl acetate (500 mL). The organic layer was separated, washed with water (100 mL), dried over Na₂SO₄ and concentrated to afford the title compound (8.12 g, 100%) as a white solid.

ESIMS (m/z): 463.8 (M+23)

### Step 6: Preparation of (1S,2S,3S,4R,5S)-5-(4-chloro-3-(phenoxymethyl)phenyl)-1-(hydroxymethyl)-6,8-dioxabicyclo[3.2.1]octane-2,3,4-triol

To a solution of (2S,3R,4S,5S)-2-(4-chloro-3-(phenoxymethyl)phenyl)-6,6-bis(hydroxymethyl)-2-methoxytetrahydro-2H-pyran-3,4,5-triol (8.0 g, 18.16 mmol) in DCM (45 mL) under nitrogen atmosphere was added TFA (4.17 mL, 54.4 mmol) at 0° C. The reaction mixture was warmed to r.t. and stirred for 3-4 h. After the completion of the reaction as confirmed by TLC, the reaction mixture was quenched with saturated solution of sodium bicarbonate. The crude compound was extracted with DCM (100 mL). The organic layer was separated, dried over Na₂SO₄ and concentrated in vacuo to afford crude title compound (7.45 g, 100%) as a white solid which was used without further purification for the next step.

ESIMS (m/z): 409.3 (M+1), 431.4 (M+23)

### Step 7: Preparation of (1R,2S,3S,4R,5S)-1-(acetoxymethyl)-5-(4-chloro-3-(phenoxymethyl)phenyl)-6,8-dioxabicyclo[3.2.1]octane-2,3,4-triyl triacetate

To a solution of (1S,2S,3S,4R,5S)-5-(4-chloro-3-(phenoxymethyl)phenyl)-1-(hydroxymethyl)-6,8-dioxabicyclo[3.2.1]octane-2,3,4-triol (7.0 g, 17.13 mmol) in THF (80 mL) under nitrogen atmosphere, was added DMAP (750.4 mg, 6.13 mmol), DIPEA (22.3 mL, 125.16 mmol) and acetic anhydride (12.88 mL, 136.58 mmol) at 0° C. The reaction mixture was warmed to r.t. and stirred for 2 h. After the completion of reaction as confirmed by TLC, the crude compound was extracted with ethyl acetate (600 mL). The organic layer was washed with 10% HCl (200 mL), separated, dried over Na₂SO₄ and concentrated in vacuo to afford crude compound which was purified by column chromatography (silica gel, 2:8 Ethyl acetate:Pet. Ether) to afford the title compound (8.2 g, 82%) as a white solid.

ESIMS (m/z): 601.9 (M+23)

### Step 8: Preparation of (1R,2S,3S,4R,5S)-1-(acetoxymethyl)-5-(3-(bromomethyl)-4-chlorophenyl)-6,8-dioxabicyclo[3.2.1]octane-2,3,4-triyl triacetate

To a solution of (1R,2S,3S,4R,5S)-1-(acetoxymethyl)-5-(4-chloro-3-(phenoxymethyl)phenyl)-6,8-dioxabicyclo[3.2.1]octane-2,3,4-triyl triacetate (5.5 g, 9.54 mmol), in acetic acid (23.62 mL) under nitrogen atmosphere, was added 37% HBr/AcOH (47.7 mL). The reaction mixture was stirred at r.t. for 3 h. After the completion of the reaction as confirmed by TLC, the reaction mixture was cooled to 0° C. and quenched very slowly with saturated solution of potassium carbonate until effervescence ceases. The crude compound was extracted with ethyl acetate (2×300 mL). The organic layer was separated, dried over Na₂SO₄ and concentrated in vacuo to afford crude compound which was purified by column chromatography (silica gel, 3:7 Ethyl acetate:Pet ether) to afford the title compound (4.28 g, 79%) as a white solid.

### Step 9: Preparation of (1R,2S,3S,4R,5S)-1-(acetoxymethyl)-5-(3-(4-acetylbenzyl)-4-chlorophenyl)-6,8-dioxabicyclo[3.2.1]octane-2,3,4-triyl triacetate

To a mixture of (1R,2S,3S,4R,5S)-1-(acetoxymethyl)-5-(3-(bromomethyl)-4-chlorophenyl)-6,8-dioxabicyclo[3.2.1]octane-2,3,4-triyl triacetate (2.7 g, 4.79 mmol), 1-(4-(4,4,5,5-tetramethyl-1,3,2-dioxaborolan-2-yl)phenyl)ethanone (1.76 g, 23.14 mmol), Pd(PPh₃)₄ (442 mg, 0.382 mmol) and cesium fluoride (2.17 g, 14.31 mmol) under nitrogen atmosphere, was added 1,4-dioxane (100 mL). The reaction mixture was refluxed at 120° C. for 2-3 h. After the completion of the reaction as confirmed by TLC, the solution was cooled to r.t. and filtered through celite. The solvent was concentrated in vacuo. The crude compound was extracted with ethyl acetate (200 mL). The organic layer was washed with water, separated, dried over Na₂SO₄ and concentrated in vacuo to afford crude compound which was purified by column chromatography (silica gel, 2:8 Ethyl acetate:Pet. Ether) to afford the title compound (820 mg, 30%) as a white solid.

ESIMS (m/z): 625.4 (M+23)

### Step 10: Preparation of (1R,2S,3S,4R,5S)-1-(acetoxymethyl)-5-(4-chloro-3-(4-(1-(ethoxyimino)ethyl)benzyl)phenyl)-6,8-dioxabicyclo[3.2.1]octane-2,3,4-triyl triacetate

To a solution of (1R,2S,3S,4R,5S)-1-(acetoxymethyl)-5-(3-(4-acetylbenzyl)-4-chlorophenyl)-6,8-dioxabicyclo[3.2.1]octane-2,3,4-triyl triacetate (800 mg, 1.32 mmol), in ethanol (20 mL) under nitrogen atmosphere, was added pyridine (0.53 mL, 6.59 mmol) and O-ethylhydroxylamine hydrochloride (244.0 mg, 2.64 mmol). The reaction mixture was refluxed at 80° C. for 4 h. After completion of the reaction as confirmed by TLC, the solvent was evaporated. The crude compound was extracted with ethyl acetate (2×50 mL). The organic layer was separated, washed with 5% HCl (20 mL), water (20 mL), dried over Na₂SO₄ and concentrated in vacuo to afford crude product which was purified by column chromatography (silica gel, 2:8 Ethyl acetate:Pet. Ether) to afford the title compound (742 mg, 80%) as a white solid.

ESIMS (m/z): 646.6 (M+1)

### Step 11: Preparation of 1-(4-(2-chloro-5-((1S,2S,3S,4R,5S)-2,3,4-trihydroxy-1-(hydroxymethyl)-6,8-dioxabicyclo[3.2.1]octan-5-yl)benzyl)phenyl)ethanone O-ethyl oxime

To a solution of (1R,2S,3S,4R,5S)-1-(acetoxymethyl)-5-(4-chloro-3-(4-(1-(ethoxyimino)ethyl)benzyl)phenyl)-6,8-dioxabicyclo[3.2.1]octane-2,3,4-triyl triacetate (600 mg, 0.93 mmol), in THF (3.08 mL), methanol (4.67 mL) and water (1.5 mL), was added lithium hydroxide monohydrate (47.12 mg, 1.12 mmol) at 0° C. The reaction was stirred at r.t. for 1 h. After the completion of reaction as confirmed by TLC, volatiles were evaporated in vacuo and the compound was extracted with ethyl acetate (100 mL). The organic layer was separated, washed with water (50 mL), dried over Na₂SO₄ and concentrated in vacuo to afford the title compound (375 mg, 84%) as a white solid.

ESIMS (m/z): 478.1 (M+1)

¹H NMR (400 MHz, CD₃OD): δ 1.28 (t, J=7.2 Hz, 3H), 2.17 (s, 3H), 3.53 (d, J=8.0 Hz, 1H), 3.58 (dd, J=7.2 and 1.2 Hz, 1H), 3.64 (t, J=7.6 Hz, 1H), 3.67 (d, J=12.8 Hz, 1H), 3.77 (dd, J=8.2 and 1.2 Hz, 1H), 3.82 (d, J=12.4 Hz, 1H), 4.11 (s, 2H), 4.13 (d, J=7.6 Hz, 1H), 4.17 (q, J=7.2 Hz, 2H), 7.19 (d, J=8.4 Hz, 2H), 7.36 (d, J=8.0 Hz, 1H), 7.40 (dd, J=8.4 and 2.0 Hz, 1H), 7.48 (d, J=2.0 Hz, 1H), 7.54 (d, J=8.4 Hz, 2H)

The compounds listed in Tables 2 to 4 were prepared essentially following the procedures described for Examples 1 to 8:

TABLE 2

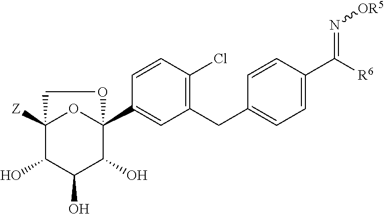

| Example No. | Z | R⁵ | R⁶ | ESIMS (m/z) |
|---|---|---|---|---|
| 9 | CH₂OH | CH₃ | H | 450.9 (M + 1) |
| 10 | CH₂OH | CH(CH₃)₂ | CH₃ | 492.6 (M + 1) |
| 11 | CH₂OH | C₂H₅ | C₂H₅ | 492.5 (M + 1) |
| 12 | CH₂OH | CH(CH₃)₂ | C₂H₅ | 506.8 (M + 1) |
| 13 | CH₂OCOOCH₂CH₃ | CH₃ | C₂H₅ | 550.2 (M + 1) |
| 14 | CH₂OCOOCH₂CH₃ | C₂H₅ | CH₃ | 550.2 (M + 1) |
| 15 | CH₂OCOCH₂CH₂CH₃ | CH₃ | C₂H₅ | 548.3 (M + 1) |
| 16 | CH₂OCOCH₂CH₂CH₃ | C₂H₅ | CH₃ | 548.3 (M + 1) |
| 17 | CH₂OH | H | C₂H₅ | 464.5 (M + 1) |

TABLE 3

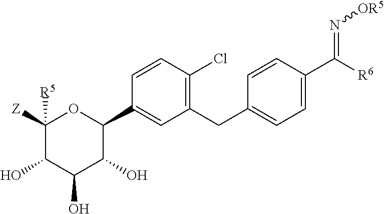

| Example | | | | | | ESIMS |
|---|---|---|---|---|---|---|
| No. | Z | R² | R⁵ | R⁶ | R⁸ | (m/z) |
| 18 | CH₂OH | Cl | CH₃ | H | H | 422.7 (M + 1) |
| 19 | CH₂OH | Cl | CH(CH₃)₂ | CH₃ | H | 464.5 (M + 1) |
| 20 | CH₂OH | Cl | (CH₂)₂OCH₃ | CH₃ | H | 480.7 (M + 1) |
| | | | | | | 481.9 (M + 2) |
| 21 | CH₂OH | Cl | 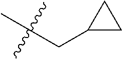 [SMILES: *CC1CC1] | CH₃ | H | 476.6 (M + 1) 498.8 (M + 23) |
| 22 | CH₂OH | Cl | 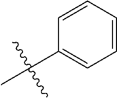 [SMILES: *c1ccccc1] | CH₃ | H | 666.8 (M + 1) |
| 23 | CH₂OH | Cl | 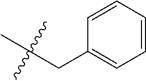 [SMILES: *Cc1ccccc1] | CH₃ | H | 512.6 (M + 1) 535.9 (M + 23) |
| 24 | CH₂OH | Cl | C₂H₅ | C₂H₅ | H | 464.5 (M + 1) |
| | | | | | | 465.8 (M + 2) |
| 25 | CH₂OH | Cl | CH(CH₃)₂ | C₂H₅ | H | 478.4 (M + 1) |
| | | | | | | 480.5 (M + 2) |
| 26 | CH₂OH | Cl | CH₃ | CH(CH₃)₂ | H | 464.6 (M + 1) |
| 27 | CH₂OH | Cl | CH₃ | (CH₂)₃CH₃ | H | 478.8 (M + 1) |
| 28 | CH₂OH | Cl | C₂H₅ | (CH₂)₃CH₃ | H | 478.3 (M + 1) |
| 29 | CH₂OH | Cl | C₂H₅ | CH(CH₃)₂ | H | 478.6 (M + 1) |
| 30 | CH₂OH | Cl | CH(CH₃)₂ | (CH₂)₃CH₃ | H | 492.3 (M + 1) |
| 31 | CH₂OH | Cl | CH(CH₃)₂ | CH(CH₃)₂ | H | 492.6 (M + 1) |
| 32 | CH₂OH | Cl | H | NH₂ | H | 423.8 (M + 1) |
| 33 | CH₂OH | Cl | CH₃ | 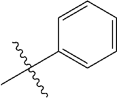 [SMILES: *c1ccccc1] | H | 498.7 (M + 1) |
| 34 | CH₂OH | Cl | CH₃ | 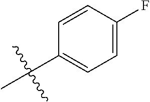 [SMILES: *c1ccc(F)cc1] | H | 516.8 (M + 1) |
| 35 | CH₂OH | Cl | CH₃ | 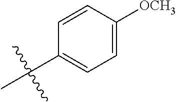 [SMILES: *c1ccc(OC)cc1] | H | 528.9 (M + 1) |
| 36 | CH₂OH | Cl | CH₃ | 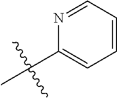 [SMILES: *c1ccccn1] | H | 499.4 (M + 1) 521.7 (M + 23) |
| 37 | CH₂OCOOCH₂CH₃ | Cl | CH₃ | C₂H₅ | H | 522.2 (M + 1) |
| 38 | CH₂OCOCH₂CH₂CH₃ | Cl | CH₃ | C₂H₅ | H | 520.2 (M + 1) |
| 39 | CH₂OH | H | CH₃ | CH₃ | H | 402.5 (M + 1) |
| 40 | CH₂OH | Cl | CH₃ | CH₃ | CH₂OH | 466.4 (M + 1) |
| 41 | CH₂OH | Cl | H | C₂H₅ | H | 436.6 (M + 1) |

TABLE 4

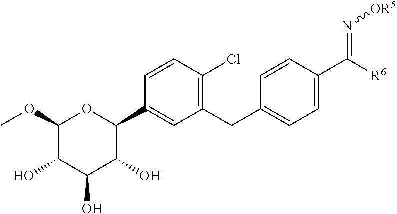

| Example No. | —R⁵ | —R⁶ | ESIMS (m/z) |
|---|---|---|---|
| 42 | CH(CH₃)₂ | CH₃ | 464.5 (M + 1) |
| 43 | 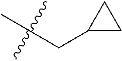 [SMILES: *CC1CC1] | CH₃ | 476.5 (M + 1) |
| 44 | 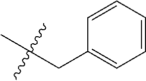 [SMILES: *Cc1ccccc1] | CH₃ | 512.6 (M + 1) |

The SGLT inhibitory effects of the compounds of the present invention were demonstrated by following test procedures.

### In Vitro Studies

Preparation of Mouse SGLT-2 Expressing Cells

Full-length mouse SGLT-2 cDNA was amplified from C57BL/6J mouse kidneys and introduced in the pcDNA3.1(+) expression vector (Invitrogen, Inc.) and propagated in Escherichia coli strain DH5α using Luria-Bertani (LB) medium containing ampicillin. Mouse SGLT-2 recombinant expression plasmid DNA was transfected into CHO-K1 cells (American Type Culture Collection) using Superfect Transfection Reagent according to a manufacturer suggested protocol. Stably transfected cells were selected using G418 antibiotic selection pressure.

Methyl-α-D-[U-¹⁴C]Glucopyranoside Uptake Assay for SGLT-2

Cells expressing mSGLT-2 were seeded on 96-well tissue culture plates (Greiner, Inc.) in RPMI containing 10% FBS and 400 μg/mL G418 (0.8×10⁵ cells per well in 200 μL medium) and incubated at 37° C. under 5% carbon dioxide for 24 h prior to the assay. Cells were washed twice with 200 μL of either sodium buffer (140 mM NaCl, 4.7 mM KCl, 2.2 mM CaCl₂, 1.2 mM MgCl₂, 10 mM Tris/Hepes, pH 7.4) or sodium-free buffer (137 mM N-methyl-glucamine, 4.7 mM KCl, 2.2 mM CaCl₂, 1.2 mM MgCl₂, 10 mM Tris/Hepes, pH 7.4). Reaction mixture containing test compounds at different concentrations diluted in assay buffer, 0.1 mM unlabeled Methyl-α-D-glucopyranoside and 1 μCi/well methyl-α-D[U-¹⁴C]glucopyranoside (American Radiochemicals) was added per well of a 96-well plate and incubated at 37° C. for 1 h. Cells were washed thrice with 200 μL of wash buffer (140 mM NaCl, 4.7 mM KCl, 2.2 mM CaCl₂, 1.2 mM MgCl₂, 10 mM Tris/Hepes, pH 7.4 containing 500 μM phlorizin) and lysed using 50 μL of 0.25N NaOH. Methyl-α-D[U-¹⁴C]glucopyranoside uptake was quantitated using a Top count scintillation counter (PerkinElmer, Inc.). All the test compounds were assayed in triplicates.

TABLE 5

| | | % inhibition of Methyl-α-D-[U-¹⁴C] | | | |
|---|---|---|---|---|---|
| | | Glucopyranoside uptake (CHO-K1 cells) | | | |
| S. | Compound | mediated by SGLT-2 | | | |
| No. | No. | 0.1 nM | 1 nM | 10 nM | 100 nM |
| 1. | 2 | 16.73 | 26.19 | 73.13 | 92.85 |
| 2. | 3 | 26.42 | 26.10 | 59.52 | 93.34 |
| 3. | 5 | 23.91 | 45.81 | 50.06 | 91.98 |
| 4. | 6 | 44.05 | 45.09 | 62.06 | 89.78 |
| 5. | 7 | 44.36 | 48.64 | 42.26 | 74.40 |
| 6. | 8 | N.E. | 25.35 | 72.44 | 90.93 |
| 7. | 9 | 14.75 | 23.16 | 65.69 | 92.16 |
| 8. | 10 | 9.23 | 20.18 | 58.35 | 87.36 |
| 9. | 11 | 36.04 | 47.42 | 48.74 | 78.08 |
| 10. | 13 | 21.63 | 25.04 | 27.26 | 85.34 |
| 11. | 14 | N.E. | N.E. | N.E. | 40.24 |
| 12. | 15 | N.E. | N.E. | 22.64 | 24.19 |
| 13. | 16 | N.E. | 18.84 | 60.74 | 91.0 |
| 14. | 26 | 39.21 | 42.27 | 44.55 | 44.99 |
| 15. | 27 | 39.21 | 41.68 | 47.47 | 45.47 |
| 16. | 29 | 32.92 | 48.69 | 40.40 | 33.61 |
| 17. | 30 | 22.95 | 25.27 | 24.41 | 42.50 |
| 18. | 31 | 36.95 | 41.44 | 42.59 | 32.65 |
| 19. | 32 | 19.02 | 26.82 | 42.90 | 29.80 |

N.E. = Not Effective

Preparation of Human SGLT-1 Expressing Cells

Full-length human SGLT-1 cDNA in the pCMV-XL-Neo expression vector was obtained from Origene Corporation and propagated in Escherichia coli strain DH5α using Luria-Bertani (LB) medium containing ampicillin. Human SGLT-1 expression plasmid DNA was transfected into CHO-K1 cells (American Type Culture Collection) using Superfect Transfection Reagent according to a manufacturer suggested protocol. Stably transfected cells were selected using G418 antibiotic selection pressure.

Methyl-α-D-[U-¹⁴C]Glucopyranoside Uptake Assay for SGLT-1

Cells expressing hSGLT-1 were seeded on 96-well tissue culture plates (Greiner, Inc.) in RPMI containing 10% FBS and 800 μg/ml G418 (0.8×10⁵ cells per well in 200 μL medium) and incubated at 37° C. under 5% carbon dioxide for 24 h prior to the assay. Cells were washed twice with 200 μL of either sodium buffer (140 mM NaCl, 4.7 mM KCl, 2.2 mM CaCl₂, 1.2 mM MgCl₂, 10 mM Tris/Hepes, pH 7.4) or sodium-free buffer (137 mM N-methyl-glucamine, 4.7 mM KCl, 2.2 mM CaCl₂, 1.2 mM MgCl₂, 10 mM Tris/Hepes, pH 7.4). Reaction mixture containing test compounds diluted in assay buffer, 1 mM unlabeled Methyl-α-D-glucopyranoside and 1 μCi/well methyl-α-D-[U-¹⁴C]glucopyranoside (American Radiochemicals) was added per well of a 96-well plate and incubated at 37° C. for 1 h. Cells were washed thrice with 200 μL of wash buffer (140 mM NaCl, 4.7 mM KCl, 2.2 mM CaCl₂, 1.2 mM MgCl₂, 10 mM Tris/Hepes, pH 7.4 containing 500 μM phlorizin) and lysed using 50 μL of 0.25N NaOH. Methyl-α-D-[U-¹⁴C]glucopyranoside uptake was quantitated using a Top count scintillation counter (PerkinElmer, Inc.). All the test compounds were assayed in triplicates.

TABLE 6

| | | % inhibition of Methyl-α-D-[U-¹⁴C] | | |
|---|---|---|---|---|
| | | Glucopyranoside uptake (CHO-K1 cells) | | |
| S. | Compound | mediated by SGLT-1 | | |
| No. | No. | 100 nM | 1 μM | 10 μM |
| 1. | 2 | N.E. | 33.10 | 78.87 |
| 2. | 3 | N.E. | N.E. | 63.64 |
| 3. | 5 | N.E. | N.E. | 49.74 |
| 4. | 6 | N.E. | 22.68 | 60.02 |
| 5. | 7 | N.E. | N.E. | 56.97 |
| 6. | 8 | 20.63 | 55.66 | 88.57 |
| 7. | 9 | 18.95 | 28.59 | 87.15 |
| 8. | 10 | N.E. | N.E. | 61.04 |
| 9. | 11 | N.E. | N.E. | 64.0 |
| 10. | 13 | N.E. | N.E. | 72.27 |
| 11. | 14 | N.E. | N.E. | 67.22 |
| 12. | 15 | N.E. | 20.75 | 65.11 |
| 13. | 16 | N.E. | 16.49 | 63.97 |
| 14. | 26 | N.E. | 17.87 | 37.86 |
| 15. | 27 | N.E. | N.E. | 34 |
| 16. | 29 | N.E. | N.E. | 24.28 |
| 17. | 30 | N.E. | N.E. | 56.93 |
| 18. | 31 | N.E. | N.E. | 49.59 |
| 19. | 32 | N.E. | N.E. | 34.65 |

N.E. = Not Effective

In Vivo Studies

Estimation of Urinary Glucose in C57BL/6J Mice

C57BL/6J mice were fasted 4 h before drug treatment. Fasted mice were weighed and randomized into different groups based on their body weight (n=6). At time T₀ test compounds and standard compounds suspended in 0.25% CMC or PEG 400 formulation, were administered to respective groups and kept in metabolic cages (6 mice/cage) after dosing. Animals were fed after drug administration and urine was collected over a period of 24 h. Urinary volume was measured and urinary glucose was estimated using Merckotest Glucose Reagent (Merck Specialities Pvt. Ltd., India).

TABLE 7

| S. | Compound | UGE in mice (in mg/g of |
|---|---|---|
| No. | No. | feed consumed) at 3 mpk |
| 1. | 2 | 88.9 |
| 2. | 3 | 81.9 |
| 3. | 4 | 76.2 |
| 4. | 5 | 81.7 |
| 5. | 6 | 78.1 |
| 6. | 7 | 78.4 |
| 7. | 8 | 60.6 |
| 8. | 9 | 86.1 |
| 9. | 10 | 66.6 |
| 10. | 11 | 70.4 |
| 11. | 16 | 67.1 |
| 12. | 17 | 64.5 |

Estimation of Urinary Glucose in Rat

Rats were fasted overnight (14 hrs) before drug treatment. Fasted rats were weighed and randomized into different groups based on their body weight (n=4). At time Tₒ test compounds and standard compound suspended in 0.25% C.M.C. or PEG 400 formulation were administered to respective groups and kept the animals in metabolic cages (1 rat/cage) after dosing. Animals were fed after drug administration and urine was collected over a period of 24 hrs. Urine volume was measured and urinary glucose was estimated using Merckotest Glucose Reagent (Merck Specialities Pvt. Ltd., India).

TABLE 8

| S. | Compound | UGE in rat (in mg/g of |
|---|---|---|
| No. | No. | feed consumed) at 3 mpk |
| 1. | 2 | 68.6 |
| 2. | 3 | 67.4 |
| 3. | 9 | 110.9 |
| 4. | 16 | 89.7 |

1. A compound of Formula I, 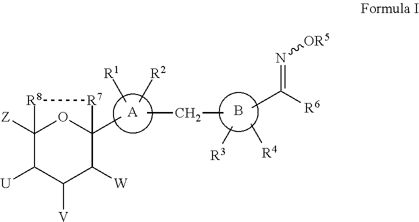
wherein:
‘---’ is either a single bond or absent;
ring A represents monocyclic or polycyclic C₃₋₂₀ cycloalkyl, C₆₋₁₀ aryl, 5-10 membered heteroaryl or 3-14 membered heterocyclyl ring;
ring B represents monocyclic or polycyclic C₃₋₂₀ cycloalkyl, C₆₋₁₀ aryl, 5-10 membered heteroaryl or 3-14 membered heterocyclyl ring;
U, V and W are independently selected from —H, C₁₋₁₂ alkyl, C₂₋₁₂ alkenyl, C₂₋₁₂ alkynyl, —OH, —CN, —N₃, —NO₂, —OCONH₂, —F, —Cl, —Br, —I, —COOH, —CONH₂, —CONHNH₂, —NH₂, —NHCONH₂, —NHCSNH₂, —NH(C═NH)NH₂, —NHNH₂, —NHCHO, —NHCOOH, —SH, —SO₃H, —CH(═NOH), —COR^{a}, —OR⁹, —COOR^{a}, —CONR^{a}R^{b}, —NR^{a}R^{b}, —NR^{a}SO₂R^{b}, —NR^{a}CONR^{b}R^{c}, —NR^{a}COR^{b}, —NR^{a}COOR^{b}, —OCOR^{a}, —OCOOR^{a}, —OCONR^{a}R^{b}, —SR^{a}, —S(O)R^{a}, —S(O)₂R^{a}, —SO₂NR^{a}R^{b}, —CR^{a}(═NOR^{b}), —NHP(O)R^{a}R^{b}; wherein the said C₁₋₁₂ alkyl, C₂₋₁₂ alkenyl and C₂₋₁₂ alkynyl, may optionally be substituted at any available position by one or more suitable substituents selected from R¹⁰;
provided that at least two out of U, V and W represent —OR⁹;
Z represents —(CH₂)ₙOR^{a}, —OR^{a}, —OCOR^{a}, —OCOOR^{a}, —OCONR^{a}R^{b}, C₁₋₁₂ alkyl, C₂₋₁₂ alkenyl, C₂₋₁₂ alkynyl, —CN, —OCONH₂, —CHO, —COOH, —CONH₂, —CONHNH₂, —NH₂, —NHCOOH, —CH₂OH, —OH, —SH, —SO₃H, —CH(═NOH), —CH(═NCN), —COR^{a}, —COOR^{a}, —CONR^{a}R^{b}, —NR^{a}R^{b}, —NR^{a}SO₂R^{b}, —NR^{a}CONR^{b}R^{c}, —NR^{a}NR^{b}R^{c}, —NR^{a}COR^{b}, —NR^{a}COOR^{b}, —SR^{a}, —S(O)R^{a}, —S(O)₂R^{a}, —SO₂NR^{a}R^{b} or —CR^{a}(═NOR^{b}); wherein the said C₁₋₁₂ alkyl, C₂₋₁₂ alkenyl and C₂₋₁₂ alkynyl, may optionally be substituted at any available position by one or more suitable substituents selected from R¹⁰;
R¹, R², R³ and R⁴ are independently selected from —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCONH₂, —F, —Cl, —Br, —I, —CHO, —COOH, —CONH₂, —NH₂, —NHCONH₂, —NHCHO, —NHCOOH, —OH, —OR^{a}, —SH, —SO₃H; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁵ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, or 5-10 membered heteroaryl; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁶ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —NH₂, —NHCONH₂, —NHCHO, —OH, —SH, —NR^{a}R^{b}, —NR^{a}CONR^{b}R^{c}, —NR^{a}COR^{b}, —OR^{a} or —SR^{a}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁷ represents —H, —OH or —OR⁹;
R⁸ represents —H, —CHO, —COOH, —CONH₂, —OH, —CH(═NOH), —COR^{a}, —COOR^{a}, —CONR^{a}R^{b}, —CR^{a}(═NOR^{b}), —OR^{a}, or —(CH₂)ₙOR^{a}; or
R⁷ and R⁸ can be joined together to form a saturated or unsaturated ring, in which one or more methylene groups or methyne groups can be replaced with O, S, NR^{a} or oxo; the ring thus formed may optionally be substituted at any available position by one or more suitable substituents selected from R¹¹; or
R⁸ and Z can be joined together to form a saturated or unsaturated ring, in which one or more methylene groups or methyne groups can be replaced with O, S, NR^{a} or oxo; the ring thus formed may optionally be substituted at any available position by one or more suitable substituents selected from R¹¹;
R⁹ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CHO, —CONH₂, —COR^{a}, —CONR^{a}R^{b}, —S(O)₂R^{a}, SO₂NR^{a}R^{b}, —P(O)R^{a}R^{b}, —P(O)OR^{a}OR^{b}, —P(O)R^{a}OR^{b}, —P(O)NR^{a}OR^{b} or —P(O)NR^{a}R^{b}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CHO, —CONH₂, —COR^{a}, —CONR^{a}R^{b}, —S(O)₂R^{a}, —SO₂NR^{a}R^{b}, —P(O)R^{a}R^{b}, —P(O)OR^{a}OR^{b}, —P(O)R^{a}OR^{b}, —P(O)NR^{a}OR^{b}, —P(O)NR^{a}R^{b}; further wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹⁰ represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH), —CH(═NCN), —COR^{a}, —CSR^{a}, —COOR^{a}, —CSOR^{a}, —COSR^{a}, —CONR^{a}R^{b}, —CSNR^{a}R^{b}, —COCOR^{a}, —CONR^{a}NR^{b}R^{c}, —CSNR^{a}NR^{b}R^{c}, —CSNR^{a}R^{b}, —NR^{a}R^{b}, —NR^{a}SO₂R^{b}, —NR^{a}CONR^{b}R^{c}, —NR^{a}CSNR^{b}R^{c}, —NR^{a}(C═NR^{b})NR^{c}R^{d}, —NR^{a}NR^{b}R^{c}, —NR^{a}COR^{b}, —NR^{a}CSR^{b}, —NR^{a}COOR^{b}, —NR^{a}CSOR^{b}, —OR^{a}, —OCOR^{a}, —OCOOR^{a}, —OCONR^{a}R^{b}, —OCSR^{a}, —OCSOR^{a}, —ONO₂, —OCSNR^{a}R^{b}, —SR^{a}, —S(O)R^{a}, —S(O)₂R^{a}, —SO₂NR^{a}R^{b}, —CR^{a}(═NOR^{b}), —CR^{a}(═NCOOR^{b}), —CR^{a}(═NSOR^{b}), —CR^{a}(═NSO₂R^{b}), —C(═NR^{a})—NR^{b}R^{c}, —C(═NOR^{a})—NR^{b}R^{c}, —CR^{a}(═NCN), —NCR^{a}, —P(O)R^{a}R^{b}, —P(O)OR^{a}OR^{b}, —P(O)R^{a}OR^{b}, —P(O)NR^{a}OR^{b}, —P(O)NR^{a}R^{b}, —OP(O)R^{a}R^{b} or —NHP(O)R^{a}R^{b}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹¹ represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH) or —CH(═NCN); wherein the C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹² represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH) or —CH(═NCN);
R^{a}, R^{b}, R^{c} and R^{d} are independently selected from —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH), —CH(═NCN); each of which may optionally be substituted at any available position by one or more suitable substituents selected from R¹²; or
R^{a} and R^{b} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹; or
R^{b} and R^{c} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹; or
R^{c} and R^{d} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹;
n represents 1, 2, 3, 4 or 5; its pharmaceutically acceptable derivatives, tautomeric forms, isomers, polymorphs, prodrugs, metabolites, salts or solvates thereof.

2. The compound according to claim 1 having the Formula Ia, 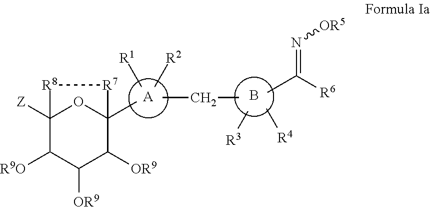
wherein:
Z, R¹, R², R³, R⁴, R⁵, R⁶, R⁷, R⁸, R⁹, ring A and ring B are as defined in claim 1; R⁹ is preferably hydrogen.

3. The compound according to claim 1 having the Formula Ib, 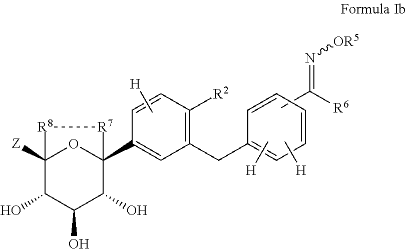
wherein:
Z, R¹, R², R³, R⁴, R⁵, R⁶, R⁷ and R⁸ are as defined in claim 1; preferably R² is Cl, F, CH₃, H, CN, cyclopropyl or ethynyl.

4. The compound according to claim 1 having the Formula Ic, 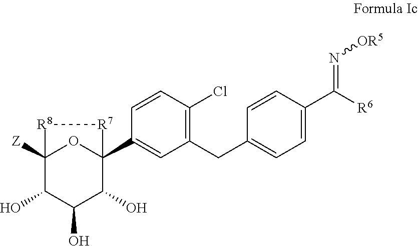
wherein:
Z, R⁵, R⁶, R⁷ and R⁸ are as defined in claim 1.

5. The compound according to claim 1,
wherein:
Z is —(CH₂)ₙOR^{a} or —OR^{a};

6. The compound according to claim 1 having the Formula Id, 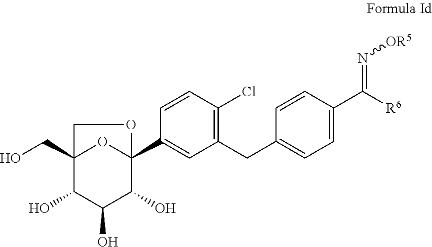
wherein:
R⁵ and R⁶ are as defined in claim 1.

7. The compound according to claim 1 having the Formula Ie, 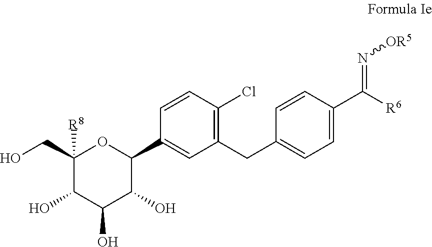
wherein:
R⁵, R⁶ and R⁸ are as defined in claim 1; preferably R⁸ is H or —CH₂OH.

8. The compound according to claim 1 having the Formula If, 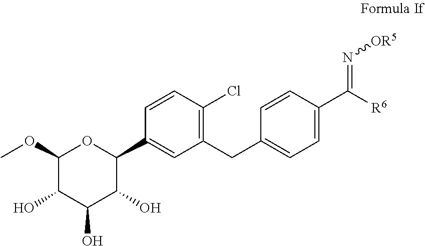
wherein:
R⁵ and R⁶ are as defined in claim 1.

9. A compound which is selected from the group consisting of: 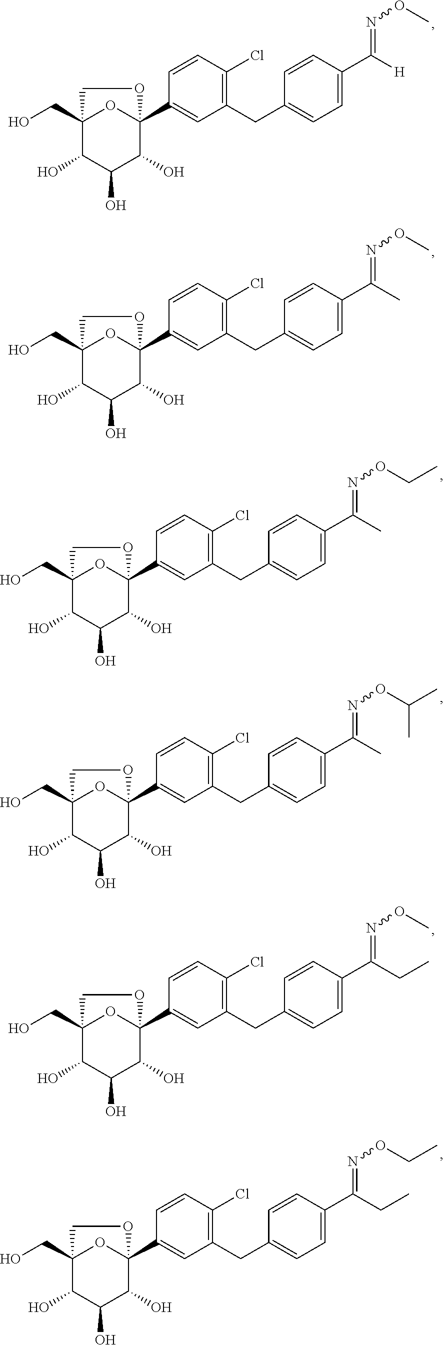 [SMILES: CON=Cc1ccc(Cc2cc([C@]34OC[C@](CO)(O3)[C@@H](O)[C@H](O)[C@H]4O)ccc2Cl)cc1 ; CON=C(C)c1ccc(Cc2cc([C@]34OC[C@](CO)(O3)[C@@H](O)[C@H](O)[C@H]4O)ccc2Cl)cc1 ; CCON=C(C)c1ccc(Cc2cc([C@]34OC[C@](CO)(O3)[C@@H](O)[C@H](O)[C@H]4O)ccc2Cl)cc1 ; CC(=NOC(C)C)c1ccc(Cc2cc([C@]34OC[C@](CO)(O3)[C@@H](O)[C@H](O)[C@H]4O)ccc2Cl)cc1 ; CCC(=NOC)c1ccc(Cc2cc([C@]34OC[C@](CO)(O3)[C@@H](O)[C@H](O)[C@H]4O)ccc2Cl)cc1 ; CCON=C(CC)c1ccc(Cc2cc([C@]34OC[C@](CO)(O3)[C@@H](O)[C@H](O)[C@H]4O)ccc2Cl)cc1] 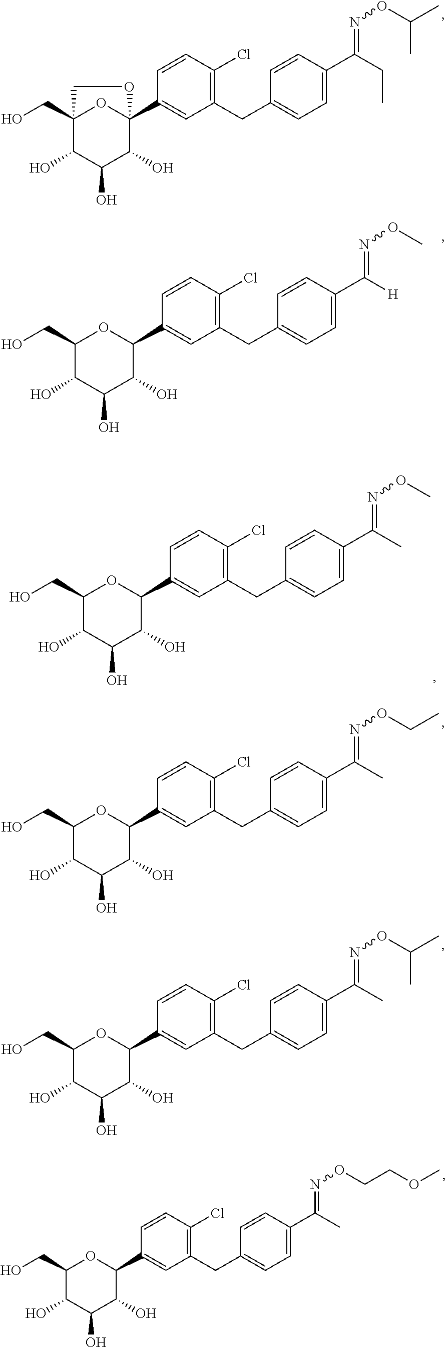 [SMILES: CCC(=NOC(C)C)c1ccc(Cc2cc([C@]34OC[C@](CO)(O3)[C@@H](O)[C@H](O)[C@H]4O)ccc2Cl)cc1 ; CON=Cc1ccc(Cc2cc([C@@H]3O[C@H](CO)[C@@H](O)[C@H](O)[C@H]3O)ccc2Cl)cc1 ; CON=C(C)c1ccc(Cc2cc([C@@H]3O[C@H](CO)[C@@H](O)[C@H](O)[C@H]3O)ccc2Cl)cc1 ; CCON=C(C)c1ccc(Cc2cc([C@@H]3O[C@H](CO)[C@@H](O)[C@H](O)[C@H]3O)ccc2Cl)cc1 ; CC(=NOC(C)C)c1ccc(Cc2cc([C@@H]3O[C@H](CO)[C@@H](O)[C@H](O)[C@H]3O)ccc2Cl)cc1 ; COCCON=C(C)c1ccc(Cc2cc([C@@H]3O[C@H](CO)[C@@H](O)[C@H](O)[C@H]3O)ccc2Cl)cc1] 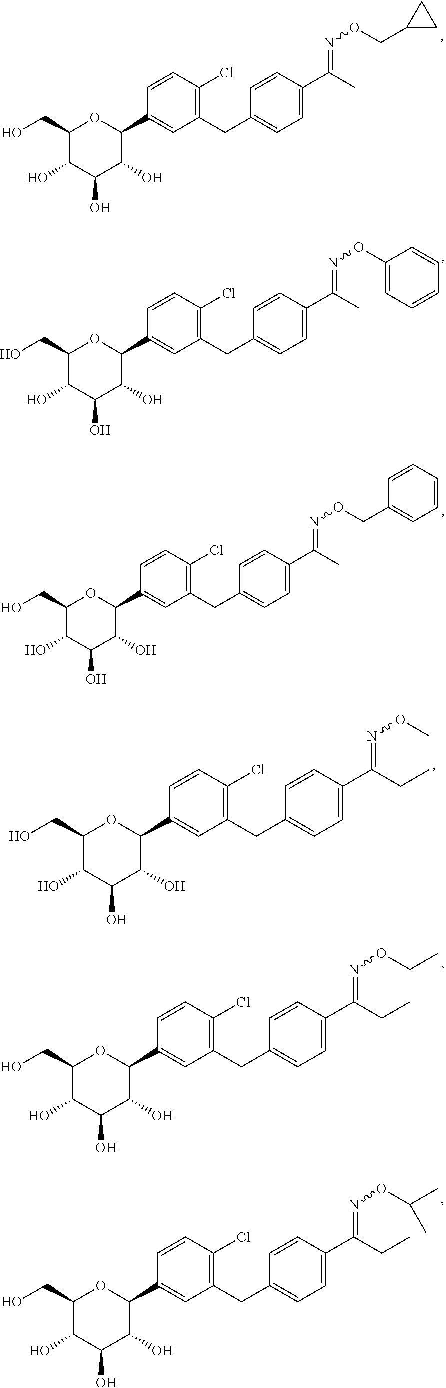 [SMILES: CC(=NOCC1CC1)c1ccc(Cc2cc([C@@H]3O[C@H](CO)[C@@H](O)[C@H](O)[C@H]3O)ccc2Cl)cc1 ; CC(=NOc1ccccc1)c1ccc(Cc2cc([C@@H]3O[C@H](CO)[C@@H](O)[C@H](O)[C@H]3O)ccc2Cl)cc1 ; CC(=NOCc1ccccc1)c1ccc(Cc2cc([C@@H]3O[C@H](CO)[C@@H](O)[C@H](O)[C@H]3O)ccc2Cl)cc1 ; CCC(=NOC)c1ccc(Cc2cc([C@@H]3O[C@H](CO)[C@@H](O)[C@H](O)[C@H]3O)ccc2Cl)cc1 ; CCON=C(CC)c1ccc(Cc2cc([C@@H]3O[C@H](CO)[C@@H](O)[C@H](O)[C@H]3O)ccc2Cl)cc1 ; CCC(=NOC(C)C)c1ccc(Cc2cc([C@@H]3O[C@H](CO)[C@@H](O)[C@H](O)[C@H]3O)ccc2Cl)cc1] 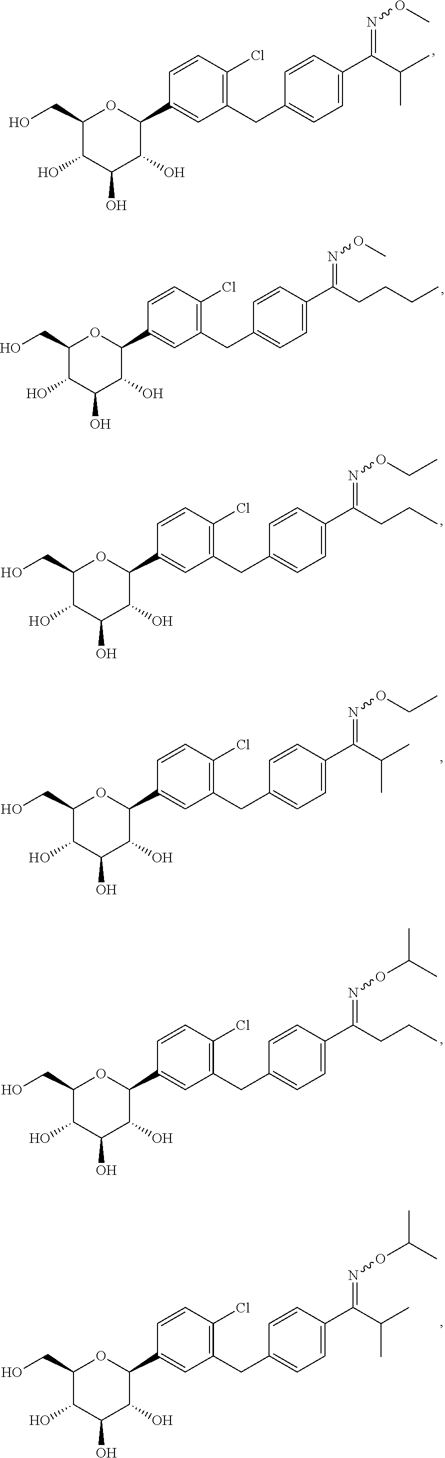 [SMILES: CON=C(c1ccc(Cc2cc([C@@H]3O[C@H](CO)[C@@H](O)[C@H](O)[C@H]3O)ccc2Cl)cc1)C(C)C ; CCCCC(=NOC)c1ccc(Cc2cc([C@@H]3O[C@H](CO)[C@@H](O)[C@H](O)[C@H]3O)ccc2Cl)cc1 ; CCCC(=NOCC)c1ccc(Cc2cc([C@@H]3O[C@H](CO)[C@@H](O)[C@H](O)[C@H]3O)ccc2Cl)cc1 ; CCON=C(c1ccc(Cc2cc([C@@H]3O[C@H](CO)[C@@H](O)[C@H](O)[C@H]3O)ccc2Cl)cc1)C(C)C ; CCCC(=NOC(C)C)c1ccc(Cc2cc([C@@H]3O[C@H](CO)[C@@H](O)[C@H](O)[C@H]3O)ccc2Cl)cc1 ; CC(C)ON=C(c1ccc(Cc2cc([C@@H]3O[C@H](CO)[C@@H](O)[C@H](O)[C@H]3O)ccc2Cl)cc1)C(C)C] 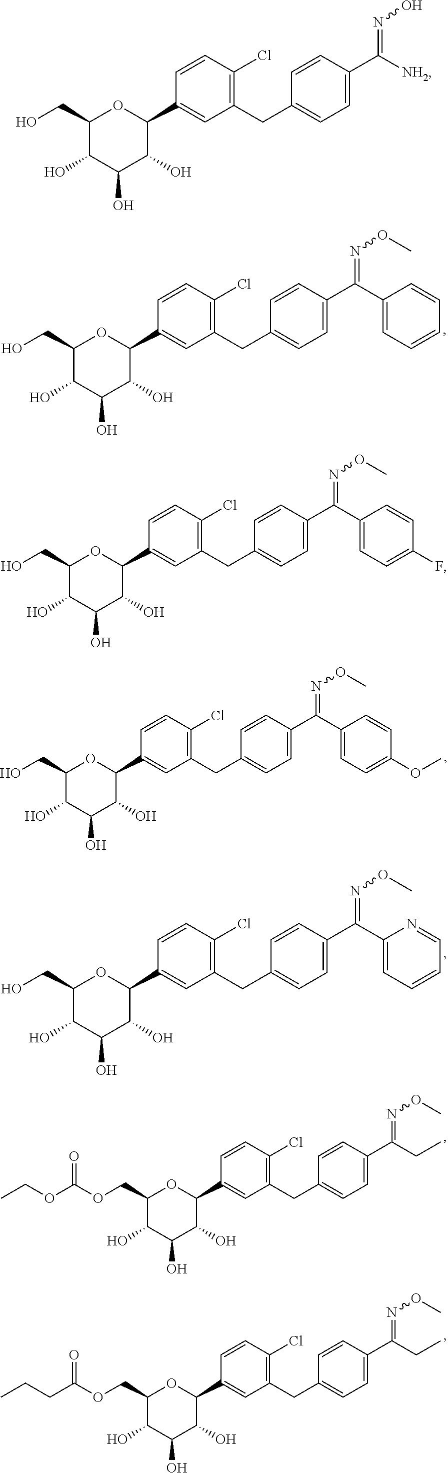 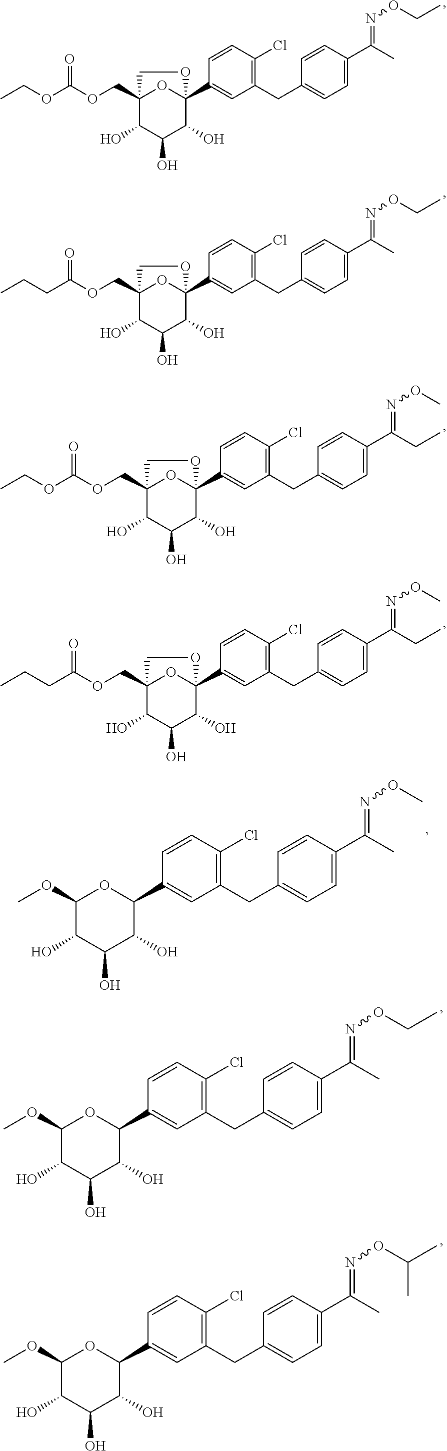 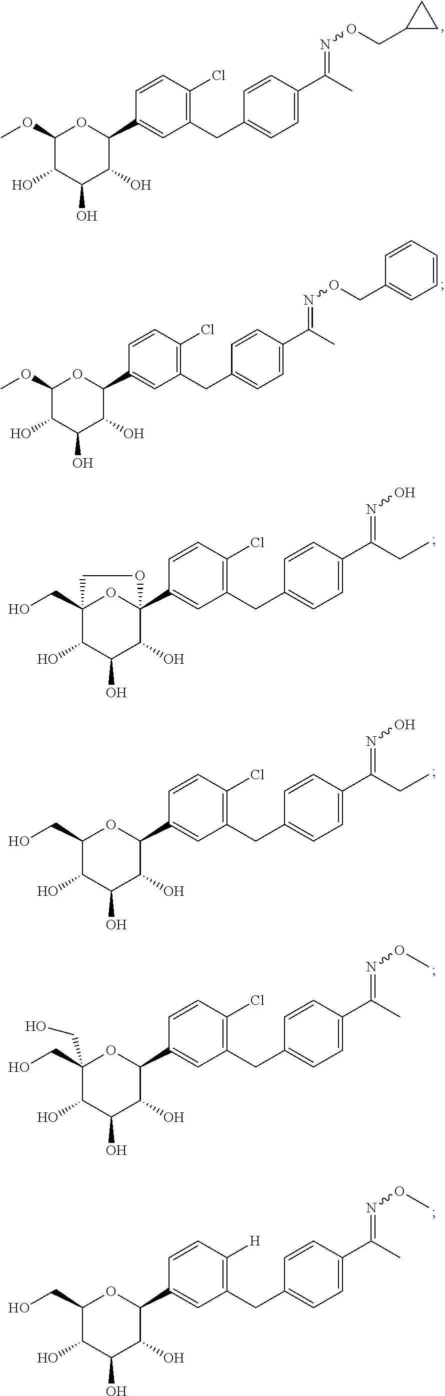 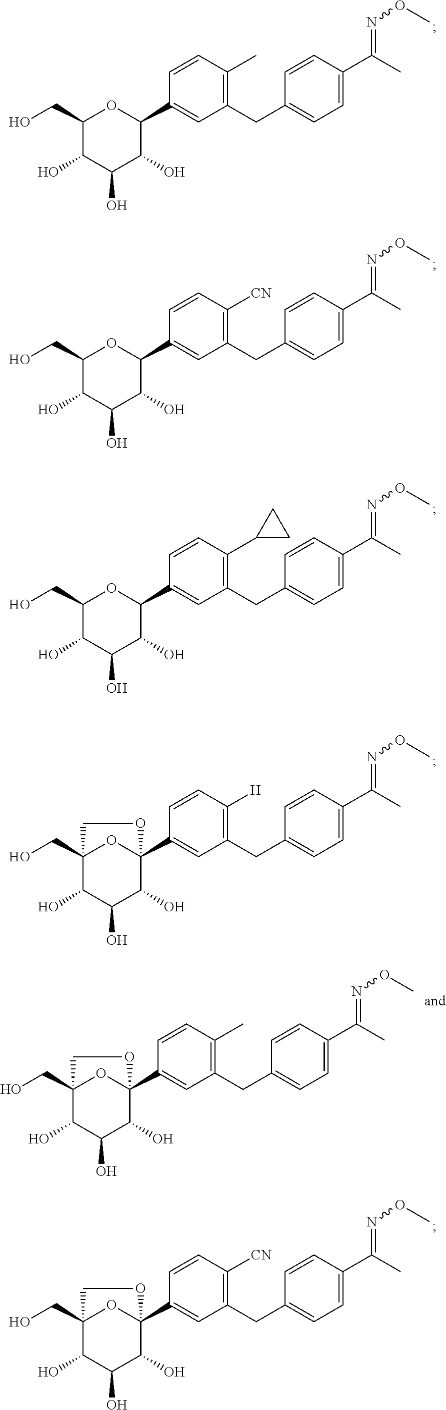
its pharmaceutically acceptable salts or solvates thereof.

10. A process for the preparation of a compound of Formula I, according to claim 1 or its pharmaceutically acceptable derivatives, tautomeric forms, isomers, polymorphs, prodrugs, metabolites, salts or solvates thereof, which comprises either of the sequences I or II:
(I) reacting compound of Formula II with compound of Formula III (R⁵ONH₂) or its salts resulting in compound of Formula I; 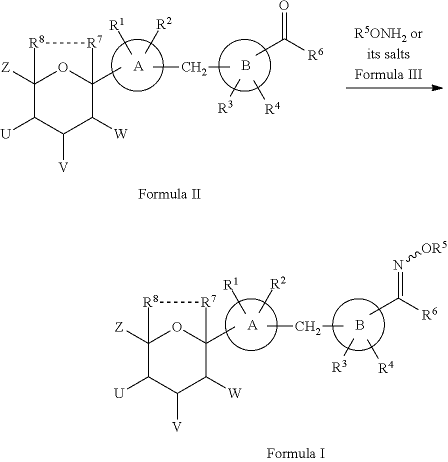
or
(II.1) reacting compound of Formula II, with compound of Formula III (R⁵ONH₂) or its salts, to obtain compound of Formula I′
(II.2) deprotecting —OPG′ of compound of Formula I′, resulting in compound of Formula I;
wherein:
R¹, R², R³, R⁴, R⁵, R⁶, R⁷, R⁸, ring A and ring B are as defined in claim 1; U, V and W are OPG′ and Z is —(CH₂)_{n′}—OPG′; OPG′ represents protected hydroxyl groups, preferably O-acetyl, O-benzyl O-allyl, O-p-methoxybenzyl and O-silyl; n′ represents 0 or 1.

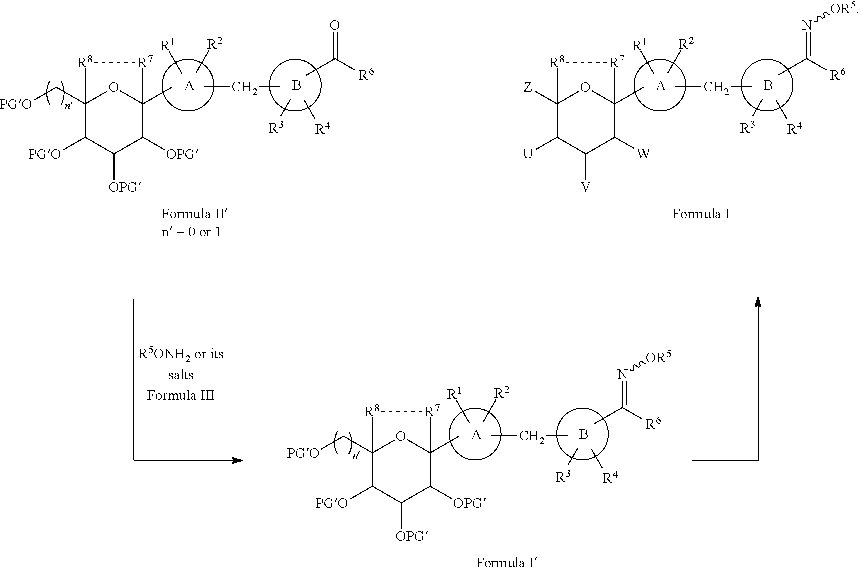

11. The compound of Formula I′, 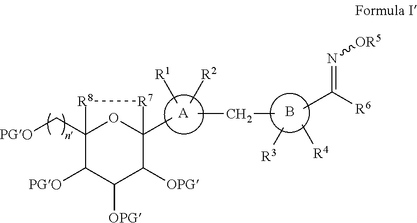
wherein:
‘---’ is either a single bond or absent;
ring A represents monocyclic or polycyclic C₃₋₂₀ cycloalkyl, C₆₋₁₀ aryl, 5-10 membered heteroaryl or 3-14 membered heterocyclyl ring;
ring B represents monocyclic or polycyclic C₃₋₂₀ cycloalkyl, C₆₋₁₀ aryl, 5-10 membered heteroaryl or 3-14 membered heterocyclyl ring;
OPG′ represents protected hydroxyl groups, preferably O-acetyl, O-benzyl O-allyl, O-p-methoxybenzyl and O-silyl;
R¹, R², R³ and R⁴ are independently selected from —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCONH₂, —F, —Cl, —Br, —I, —CHO, —COOH, —CONH₂, —NH₂, —NHCONH₂, —NHCHO, —NHCOOH, —OH, —OR^{a}, —SH, —SO₃H; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁵ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, or 5-10 membered heteroaryl; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁶ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —NH₂, —NHCONH₂, —NHCHO, —OH, —SH, —NR^{a}R^{b}, —NR^{a}CONR^{b}R^{c}, —NR^{a}COR^{b}, —OR^{a} or —SR^{a}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹⁰;
R⁷ represents —H, —OH or —OR⁹;
R⁸ represents —H, —CHO, —COOH, —CONH₂, —OH, —CH(═NOH), —COR^{a}, —COOR^{a}, —CONR^{a}R^{b}, —CR^{a}(═NOR^{b}), —OR^{a}, or —(CH₂)ₙOR^{a}; or
R⁷ and R⁸ can be joined together to form a saturated or unsaturated ring, in which one or more methylene groups or methyne groups can be replaced with O, S, NR^{a} or oxo; the ring thus formed may optionally be substituted at any available position by one or more suitable substituents selected from R¹¹;
R⁹ represents —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CHO, —CONH₂, —COR^{a}, —CONR^{a}R^{b}, —S(O)₂R^{a}, —SO₂NR^{a}R^{b}, —P(O)R^{a}R^{b}, —P(O)OR^{a}OR^{b}, —P(O)R^{a}OR^{b}, —P(O)NR^{a}OR^{b} or —P(O)NR^{a}R^{b}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CHO, —CONH₂, —COR^{a}, —CONR^{a}R^{b}, —S(O)₂R^{a}, —SO₂NR^{a}R^{b}, —P(O)R^{a}R^{b}, —P(O)OR^{a}OR^{b}, —P(O)R^{a}OR^{b}, —P(O)NR^{a}OR^{b}, —P(O)NR^{a}R^{b}; further wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹⁰ represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH), —CH(═NCN), —COR^{a}, —CSR^{a}, —COOR^{a}, —CSOR^{a}, —COSR^{a}, —CONR^{a}R^{b}, —CSNR^{a}R^{b}, —COCOR^{a}, —CONR^{a}NR^{b}R^{c}, —CSNR^{a}NR^{b}R^{c}, —CSNR^{a}R^{b}, —NR^{a}R^{b}, —NR^{a}SO₂R^{b}, —NR^{a}CONR^{b}R^{c}, —NR^{a}CSNR^{b}R^{c}, —NR^{a}(C═NR^{b})NR^{c}R^{d}, —NR^{a}NR^{b}R^{c}, —NR^{a}COR^{b}, —NR^{a}CSR^{b}, —NR^{a}COOR^{b}, —NR^{a}CSOR^{b}, —OR^{a}, —OCOR^{a}, —OCOOR^{a}, —OCONR^{a}R^{b}, —OCSR^{a}, —OCSOR^{a}, —ONO₂, —OCSNR^{a}R^{b}, —SR^{a}, —S(O)R^{a}, —S(O)₂R^{a}, —SO₂NR^{a}R^{b}, —CR^{a}(═NOR^{b}), —CR^{a}(═NCOOR^{b}), —CR^{a}(═NSOR^{b}), —CR^{a}(═NSO₂R^{b}), —C(═NR^{a})—NR^{b}R^{c}, —C(═NOR^{a})—NR^{b}R^{c}, —CR^{a}(═NCN), —NCR^{a}, —P(O)R^{a}R^{b}, —P(O)OR^{a}OR^{b}, —P(O)R^{a}OR^{b}, —P(O)NR^{a}OR^{b}, —P(O)NR^{a}R^{b}, —OP(O)R^{a}R^{b} or —NHP(O)R^{a}R^{b}; wherein the said C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹¹ represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH) or —CH(═NCN); wherein the C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl and 5-10 membered heteroaryl, may optionally be substituted at any available position by one or more suitable substituents selected from R¹²;
R¹² represents C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH) or —CH(═NCN);
R^{a}, R^{b}, R^{c} and R^{d} are independently selected from —H, C₁₋₁₂ alkyl, C₂₋₁₂ alkenyl, C₂₋₁₂ alkynyl, C₆₋₁₀ aryl, C₃₋₂₀ cycloalkyl, 3-14 membered heterocyclyl, 5-10 membered heteroaryl, —CN, —COCN, —N₃, —NO₂, —OCN, —NCO, —SCN, —NCS, —OCONH₂, —ONO₂, —F, —Cl, —Br, —I, —CO—, —CS—, —CHO, —CHS, —COOH, —COSH, —CONH₂, —CONHNH₂, —CSNHNH₂, —CSNH₂, —NH₂, —NHCONH₂, —NHCSNH₂, —NH(C═NH)NH₂, —NHNH₂, —NHCHO, —NHCHS, —NHCOOH, —NHCSOH, —OH, —SH, —SO₃H, —CH(═NOH), —CH(═NCN); each of which may optionally be substituted at any available position by one or more suitable substituents selected from R¹²; or
R^{a} and R^{b} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹; or
R^{b} and R^{c} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹; or
R^{c} and R^{d} when attached to the same atom, can be joined together to form a monocyclic or polycyclic ring, in which one or more methylene groups or methyne groups can be replaced with O, S, SO, SO₂, NR^{a}, PR^{a}, P(═O)R^{a} or oxo; the ring thus formed may optionally be substituted at any available position by one or more suitable substituents selected from R¹¹;
n represents 1, 2, 3, 4 or 5;
n′ represents 0 or 1.

12. A pharmaceutical composition, comprising a compound according to claim 1, optionally in combination with one or more pharmaceutically acceptable carrier(s).

13. A method for prophylaxis, amelioration and/or treatment of one or more condition(s)/disease(s)/disorder(s) mediated by SGLT-2, in a subject in need thereof, which comprises administering a therapeutically effective amount of compound according to claim 1.

14. A method for the prophylaxis, amelioration and/or treatment of one or more diseases, disorders and conditions selected from the group consisting of diabetes (including Type I and Type II), Metabolic Syndrome or ‘Syndrome X’ including impaired glucose tolerance, insulin resistance, metabolic acidosis or ketosis, disorders of food intake, satiety disorders, obesity, hyperinsulinemia, dyslipidemia (including hyperlipidemia, hypertriglyceridemia, hypercholesterolemia, low HDL levels, high LDL levels), hypertension associated with metabolic disorders, congestive heart failure, edema, hyperuricemia, gout, wound healing, tissue ischemia, which comprises administering a therapeutically effective amount of compound according to claim 1.

15. A method for the prophylaxis, amelioration and/or treatment of the diseases, disorders and conditions collectively referenced to as “diabetic complications” which include both acute complications and chronic complication, which comprises administering a therapeutically effective amount of compound according to claim 1.

16. Use of a compound according to claim 1, for the manufacture of a medicament for the prophylaxis, amelioration and/or treatment of one or more conditions mediated by SGLT-2, in a subject in need thereof.

17. Use of a compound according to claim 1, in combination with other therapeutic agents.

18. Use according to claim 16, wherein the medicament is administered orally, parenterally or topically.

19. (canceled)
